(19) United States
(12) Patent Application Publication
(10) Pub. No.: US 2019/0031667 A1
(43) Pub. Date: Jan. 31, 2019

(54) PESTICIDALLY ACTIVE HETEROCYCLIC DERIVATIVES WITH SULPHUR CONTAINING SUBSTITUENTS

(71) Applicant: SYNGENTA PARTICIPATIONS AG, Basel (CH)

(72) Inventor: Michel Muehlebach, Stein (CH)

(73) Assignee: SYNGENTA PARTICIPATIONS AG, Basel (CH)

(21) Appl. No.: 16/075,574

(22) PCT Filed: Feb. 1, 2017
(86) PCT No.: PCT/EP2017/052086

§ 371 Date: Aug. 3, 2018

(30) Foreign Application Priority Data
Feb. 5, 2016 (EP) 16154492.9
Nov. 2, 2016 (IN) 201611037488

(51) Int. Cl. C07D 487/04; C07D 471/04; A01N 43/90

(52) U.S. Cl. CPC C07D 487/04; A01N 43/90; C07D 471/04

(57) ABSTRACT

Compounds of formula (I) wherein the substituents are as defined in claim 1, and the agrochemically acceptable salts salts, stereoisomers, enantiomers, tautomers and N-oxides of those compounds, can be used as insecticides and can be prepared in a manner known per se.

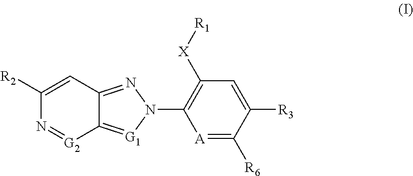

The present invention relates to pesticidally active, in particular insecticidally active heterocyclic derivatives containing sulphur substituents, to compositions comprising those compounds, and to their use for controlling animal pests (including arthropods and in particular insects or representatives of the order Acarina).

Heterocyclic compounds with pesticidal action are known and described, for example, in WO 2012/086848, WO 2013/018928, WO 2013/191112 and WO 2013/191113.

There have now been found novel pesticidally active heterocyclic derivatives with sulphur containing phenyl and pyridyl substituents.

The present invention accordingly relates to compounds of formula I,

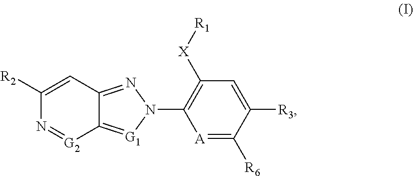

wherein

A is CH or N;

X is S, SO or SO₂;

R₁ is C₁-C₆alkyl, C₁-C₄haloalkyl, C₃-C₆cycloalkyl or C₃-C₆cycloalkyl-C₁-C₄alkyl; or

R₁ is C₃-C₆cycloalkyl mono- or polysubstituted by substituents independently selected from the group consisting of C₁-C₄alkyl, C₁-C₄haloalkyl, halogen and cyano; or

R₁ is C₃-C₆cycloalkyl-C₁-C₄alkyl mono- or polysubstituted by substituents independently selected from the group consisting of C₁-C₄alkyl, C₁-C₄haloalkyl, halogen and cyano;

R₂ is hydrogen, halogen, cyano, C₁-C₆haloalkyl, C₁-C₄haloalkylsulfanyl, C₁-C₄haloalkylsulfinyl, C₁-C₄haloalkylsulfonyl, C₁-C₄haloalkoxy or —C(O)(C₁-C₄haloalkyl); or

R₂ is C₃-C₆cycloalkyl which can be mono- or polysubstituted by substituents independently selected from the group consisting of C₁-C₄alkyl, C₁-C₄haloalkyl, halogen and cyano;

R₃ is hydrogen, C₁-C₄alkyl, C₁-C₄haloalkyl, halogen or cyano;

G₁ is CR₄, wherein R₄ is hydrogen, C₁-C₄alkyl, C₁-C₄haloalkyl, cyano or halogen;

G₂ is N or CR₅, wherein R₅ is hydrogen, C₁-C₄alkyl, C₁-C₄haloalkyl, cyano, nitro or halogen;

R₆ is amino, NHOH, NR₇R₈, C₁-C₆alkoxy, C₁-C₄alkoxyC₁-C₄alkyl, C₃-C₆cycloalkyl, C₃-C₆cycloalkyl-C₁-C₄alkyl, C₂-C₆alkenyl, C₂-C₆haloalkenyl, C₂-C₆alkynyl, C₂-C₆haloalkynyl, C₁-C₆haloalkylsulfanyl, C₁-C₆haloalkylsulfinyl, C₁-C₆haloalkylsulfonyl, C₁-C₆ haloalkoxy, —C(O)C₁-C₄haloalkyl, C₁-C₆alkylsulfanyl, C₁-C₆alkylsulfinyl, or C₁-C₆alkylsulfonyl; or

R₆ is C₅-C₆cycloalkyl which is mono- or di-substituted by substituents selected from the group consisting of halogen, C₁-C₄alkyl, C₁-C₄haloalkyl and cyano; or

R₆ is phenyl which can be mono- or polysubstituted by substituents selected from the group consisting of halogen, cyano, nitro, C₂-C₆alkenyl, C₂-C₆haloalkenyl, C₂-C₆alkynyl, C₂-C₆haloalkynyl, C₁-C₄alkyl, C₁-C₄haloalkyl, C₁-C₄haloalkoxy, C₁-C₄alkoxy, C₁-C₄alkoxy-C₁-C₄alkyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl, C₁-C₄alkylsulfonyl, and —C(O)C₁-C₄haloalkyl; or

R₆ is pyrimidinyl which can be mono- or polysubstituted by substituents selected from the group consisting of halogen, cyano, nitro, C₂-C₆alkenyl, C₂-C₆haloalkenyl, C₂-C₆alkynyl, C₂-C₆haloalkynyl, C₁-C₄alkyl, C₁-C₄haloalkyl, C₁-C₄haloalkoxy, C₁-C₄alkoxy, C₁-C₄alkoxy-C₁-C₄alkyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl, C₁-C₄alkylsulfonyl and —C(O)C₁-C₄haloalkyl; or

R₆ is pyridinyl which can be mono- or polysubstituted by substituents selected from the group consisting of halogen, cyano, nitro, C₂-C₆alkenyl, C₂-C₆haloalkenyl, C₂-C₆alkynyl, C₂-C₆haloalkynyl, C₁-C₄alkyl, C₁-C₄haloalkyl, C₁-C₄haloalkoxy, C₁-C₄alkoxy, C₁-C₄alkoxy-C₁-C₄alkyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl, or C₁-C₄alkylsulfonyl and —C(O)C₁-C₄haloalkyl; or

R₆ is a five- to six-membered, aromatic, partially saturated or fully saturated ring system linked via a nitrogen ring atom to the ring which contains the substituent X—R₁, said ring system can be mono- or polysubstituted by substituents selected from the group consisting of halogen, cyano, nitro, C₂-C₆alkenyl, C₂-C₆haloalkenyl, C₂-C₆alkynyl, C₂-C₆haloalkynyl, C₁-C₄alkyl, C₁-C₄haloalkyl, C₁-C₄haloalkoxy, C₁-C₄alkoxy, C₁-C₄alkoxy C₁-C₄alkyl, C₁-C₄alkylsulfanyl, C₁-C₄alkylsulfinyl, C₁-C₄alkylsulfonyl, —C(O)C₁-C₄alkyl, C₁-C₄haloalkylsulfanyl, C₁-C₄haloalkylsulfinyl, C₁-C₄haloalkylsulfonyl and —C(O)C₁-C₄haloalkyl; and said ring system contains 1, 2 or 3 heteroatoms selected from the group consisting of nitrogen, oxygen and sulphur, where said ring system may not contain more than one oxygen atom and not more than one sulfur atom;

R₇ is C₁-C₄alkyl, C₁-C₄haloalkyl, C₁-C₄haloalkoxy, C₁-C₄alkoxy or is C₅-C₆cycloalkyl which is mono- or di-substituted by substituents independently selected from the group consisting of halogen, C₁-C₄alkyl, C₁-C₄haloalkyl and cyano; or

R₇ is —C(O)C₁-C₄alkyl, —C(O)C₁-C₄haloalkyl or —C(O)C₂-C₆cycloalkyl; and R₈ is hydrogen, C₁-C₄alkyl, C₁-C₄haloalkyl, C₁-C₄haloalkoxy, C₁-C₄alkoxy or is C₅-C₆cycloalkyl which is mono- or di-substituted by substituents independently selected from the group consisting of halogen, C₁-C₄alkyl, C₁-C₄haloalkyl and cyano; or

R₈ is —C(O)C₁-C₄alkyl, —C(O)C₁-C₄haloalkyl or —C(O)C₂-C₆cycloalkyl;

and agrochemically acceptable salts, stereoisomers, enantiomers, tautomers and N-oxides of the compounds of formula I.

Compounds of formula I which have at least one basic centre can form, for example, acid addition salts, for example with strong inorganic acids such as mineral acids, for example perchloric acid, sulfuric acid, nitric acid, nitrose acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds of formula I which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

The alkyl groups occurring in the definitions of the substituents can be straight-chain or branched and are, for example, methyl, ethyl, n-propyl, isopropyl, n-butyl, sec-butyl, iso-butyl, tert-butyl, pentyl, hexyl, nonyl, decyl and their branched isomers. Alkylsulfanyl, alkylsulfinyl, alkylsulfonyl, alkoxy, alkenyl and alkynyl radicals are derived from the alkyl radicals mentioned. The alkenyl and alkynyl groups can be mono- or polyunsaturated.

Halogen is generally fluorine, chlorine, bromine or iodine. This also applies, correspondingly, to halogen in combination with other meanings, such as haloalkyl or halophenyl.

Haloalkyl groups preferably have a chain length of from 1 to 6 carbon atoms. Haloalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl.

Alkoxy groups preferably have a preferred chain length of from 1 to 6 carbon atoms. Alkoxy is, for example, methoxy, ethoxy, propoxy, i-propoxy, n-butoxy, isobutoxy, sec-butoxy and tert-butoxy and also the isomeric pentyloxy and hexyloxy radicals.

Alkoxyalkyl groups preferably have a chain length of 1 to 6 carbon atoms.

Alkoxyalkyl is, for example, methoxymethyl, methoxyethyl, ethoxymethyl, ethoxyethyl, n-propoxymethyl, n-propoxyethyl, isopropoxymethyl or isopropoxyethyl.

Alkylsulfanyl is for example methylsulfanyl, ethylsulfanyl, propylsulfanyl, isopropylsulfanyl, butylsulfanyl, pentylsulfanyl, and hexylsulfanyl.

Alkylsulfinyl is for example methylsulfinyl, ethylsulfinyl, propylsulfinyl, isopropylsulfinyl, a butylsulfinyl, pentylsulfinyl, and hexylsulfinyl.

Alkylsulfonyl is for example methylsulfonyl, ethylsulfonyl, propylsulfonyl, isopropylsulfonyl, butylsulfonyl, pentylsulfonyl, and hexylsulfonyl.

Alkoxycarbonyl is for example methoxycarbonyl (which is C₁alkoxycarbonyl), ethoxycarbonyl, propoxycarbonyl, isopropoxycarbonyl, n-butoxycarbonyl, tert-butoxycarbonyl, n-pentoxycarbonyl or hexoxycarbonyl.

The cycloalkyl groups preferably have from 3 to 6 ring carbon atoms, for example cyclopropyl, cyclobutyl, cyclopentyl and cyclohexyl.

Haloalkoxy groups preferably have a chain length of from 1 to 4 carbon atoms. Haloalkoxy is, for example, difluoromethoxy, trifluoromethoxy or 2,2,2-trifluoroethoxy.

Haloalkylsulfanyl groups preferably have a chain length of from 1 to 4 carbon atoms. Haloalkylsulfanyl is, for example, difluoromethylsulfanyl, trifluoromethylsulfanyl or 2,2,2-trifluoroethylsulfanyl. Similar considerations apply to the radicals C₁-C₄haloalkylsulfinyl and C₁-C₄haloalkylsulfonyl, which may be, for example, trifluoromethylsulfinyl, trifluoromethylsulfonyl or 2,2,2-trifluoroethylsulfonyl.

In the context of this invention, examples of a “five- to six-membered, aromatic, partially saturated or fully saturated ring system linked via a nitrogen atom” are, but not limited to, pyrazole, pyrrole, pyrrolidine, pyrrolidine-2-one, imidazole, triazole and pyridine-2-one.

In the context of this invention “mono- to polysubstituted” in the definition of the substituents, means typically, depending on the chemical structure of the substituents, monosubstituted to seven-times substituted, preferably monosubstituted to five-times substituted, more preferably mono-, double- or triple-substituted.

In the context of this invention “pyrimidinyl or pyridinyl” as R₆ may be both linked via any carbon atom to the ring which contains the substituent X—R₁.

The compounds of formula I according to the invention also include hydrates which may be formed during the salt formation.

Free radicals represents methyl groups.

In preferred compounds of formula I,

R₆ is amino, C₁-C₆alkoxy, C₁-C₄alkoxyC₁-C₄alkyl, C₃-C₆cycloalkyl, C₃-C₆cycloalkyl-C₁-C₄alkyl, C₂-C₆alkenyl, C₂-C₆haloalkenyl, C₂-C₆alkynyl, C₂-C₆haloalkynyl, C₁-C₆haloalkylsulfanyl, C₁-C₆haloalkylsulfinyl, C₁-C₆haloalkylsulfonyl, C₁-C₆ haloalkoxy, —C(O)C₁-C₄haloalkyl, C₁-C₆alkylsulfanyl, C₁-C₆alkylsulfinyl, or C₁-C₆alkylsulfonyl; or

R₆ is C₃-C₆cycloalkyl which is mono- or di-substituted by substituents independently selected from the group consisting of halogen, C₁-C₄alkyl, C₁-C₄haloalkyl and cyano; or

R₆ is phenyl which can be mono- or polysubstituted by substituents independently selected from the group consisting of halogen, cyano, nitro, C₂-C₆alkenyl, C₂-C₆haloalkenyl, C₂-C₆alkynyl, C₂-C₆haloalkynyl, C₁-C₄alkyl, C₁-C₄haloalkyl, C₁-C₄haloalkoxy, C₁-C₄alkoxy, C₁-C₄alkoxy C₁-C₄alkyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl, C₁-C₄alkylsulfonyl, and —C(O)C₁-C₄haloalkyl; or

R₆ is pyrimidinyl which can be mono- or polysubstituted by substituents independently selected from the group consisting of halogen, cyano, nitro, C₂-C₆alkenyl, C₂-C₆haloalkenyl, C₂-C₆alkynyl, C₂-C₆haloalkynyl, C₁-C₄alkyl, C₁-C₄haloalkyl, C₁-C₄haloalkoxy, C₁-C₄alkoxy, C₁-C₄alkoxy C₁-C₄alkyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl, C₁-C₄alkylsulfonyl and —C(O)C₁-C₄haloalkyl; or

R₆ is pyridinyl which can be mono- or polysubstituted by substituents independently selected from the group consisting of halogen, cyano, nitro, C₂-C₆alkenyl, C₂-C₆haloalkenyl, C₂-C₆alkynyl, C₂-C₆haloalkynyl, C₁-C₄alkyl, C₁-C₄haloalkyl, C₁-C₄haloalkoxy, C₁-C₄alkoxy, C₁-C₄alkoxy C₁-C₄alkyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl, or C₁-C₄alkylsulfonyl and —C(O)C₁-C₄haloalkyl; or

R₆ is a five- to six-membered, aromatic, partially saturated or fully saturated ring system linked via a nitrogen ring atom to the ring which contains the substituent X—R₁, said ring system can be mono- or polysubstituted by substituents independently selected from the group consisting of halogen, cyano, nitro, C₂-C₆alkenyl, C₂-C₆haloalkenyl, C₂-C₆alkynyl, C₂-C₆haloalkynyl, C₁-C₄alkyl, C₁-C₄haloalkyl, C₁-C₄haloalkoxy, C₁-C₄alkoxy, C₁-C₄alkoxy C₁-C₄alkyl, C₁-C₄alkylsulfanyl, C₁-C₄alkylsulfinyl, C₁-C₄alkylsulfonyl, —C(O)C₁-C₄alkyl, C₁-C₄haloalkylsulfanyl, C₁-C₄haloalkylsulfinyl, C₁-C₄haloalkylsulfonyl and —C(O)C₁-C₄haloalkyl; and said ring system contains 1, 2 or 3 heteroatoms selected from the group consisting of nitrogen, oxygen and sulphur, where said ring system may not contain more than one oxygen atom and not more than one sulfur atom.

A preferred group of compounds of formula I is represented by the compounds of formula I-1

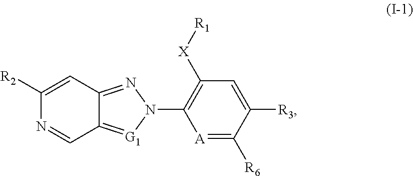

wherein the substituents X, R₁, R₂, R₃, R₆, G₁ and A are as defined under formula I above.

Further preferred embodiments of the invention are:

### Embodiment (A1)

Preferred are compounds of formula I-1, wherein

R₁ is C₁-C₄alkyl, C₃-C₆cycloalkyl-C₁-C₄alkyl or C₃-C₆cycloalkyl;

R₂ is halogen, C₁-C₄haloalkylsulfanyl, C₁-C₄haloalkylsulfinyl, C₁-C₄haloalkylsulfonyl, C₁-C₄haloalkoxy, C₁-C₄haloalkyl, cyano or is C₃-C₆cycloalkyl which can be mono- or polysubstituted by substituents selected from the group consisting of halogen, C₁-C₄ haloalkyl, cyano and C₁-C₄alkyl; and

X, R₃, R₆, G₁ and A are as defined under formula I above.

### Embodiment (A2)

Further preferred are compounds of formula I-1a

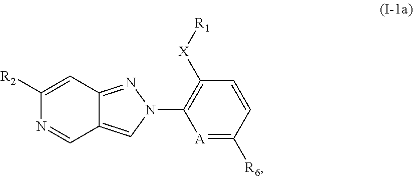

wherein

R₁ is C₁-C₄alkyl, C₃-C₆cycloalkyl-C₁-C₄alkyl or C₃-C₆cycloalkyl;

R₂ is halogen, C₁-C₄haloalkylsulfanyl, C₁-C₄haloalkylsulfinyl, C₁-C₄haloalkylsulfonyl, C₁-C₄haloalkoxy, C₁-C₄haloalkyl, cyano or is C₃-C₆cycloalkyl which can be mono- or polysubstituted by substituents independently selected from the group consisting of halogen, C₁-C₄ haloalkyl, cyano and C₁-C₄alkyl;

and

X, R₆ and A are as defined under formula I above.

### Embodiment (A3)

Further preferred are compounds of formula I-1a

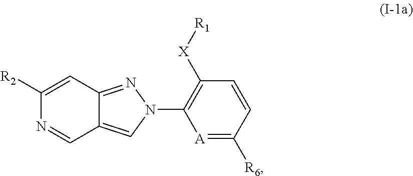

wherein

R₁ is C₁-C₄alkyl, C₃-C₆cycloalkyl-C₁-C₄alkyl or C₃-C₆cycloalkyl;

R₂ is halogen, C₁-C₄haloalkylsulfanyl, C₁-C₄haloalkylsulfinyl, C₁-C₄haloalkylsulfonyl, C₁-C₄haloalkoxy, C₁-C₄haloalkyl, cyano or is C₃-C₆cycloalkyl which can be mono- or polysubstituted by substituents selected from the group consisting of halogen, C₁-C₄ haloalkyl, cyano and C₁-C₄alkyl;

X and A are as defined under formula I above; and

R₆ is selected from the group J consisting of

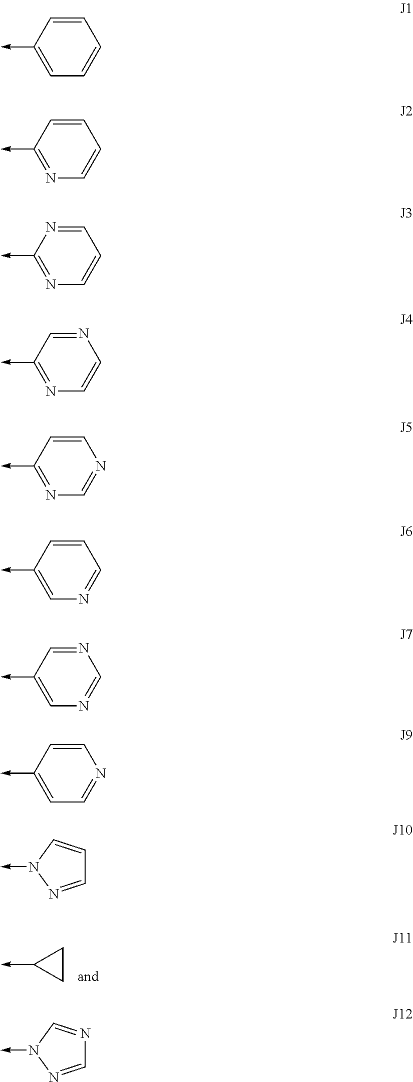

wherein each of the groups J1 to J10 and J12 independently can be mono- or di-substituted by substituents independently selected from the group consisting of halogen, cyano, nitro, C₂-C₆alkenyl, C₂-C₆haloalkenyl, C₂-C₆alkynyl, C₂-C₆haloalkynyl, C₁-C₄haloalkyl, C₁-C₄haloalkoxy, C₁-C₄alkoxy, C₁-C₄alkoxy C₁-C₄alkyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl and C₁-C₄alkylsulfonyl; and J11 can be substituted by halogen, C₁-C₄alkyl, C₁-C₄haloalkyl or cyano; or

R₆ is amino; or

R₆ is NHOH; or

R₆ is NR₇R₈, wherein R₇ is C₁-C₄alkyl and R₈ is hydrogen.

### Embodiment (A4)

Further preferred are compounds of formula I-1a

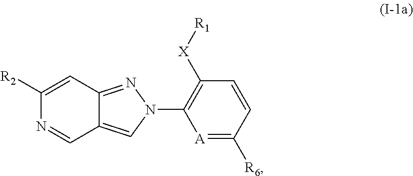

wherein R₆ is as defined as J under Embodiment (A3) above;

R₁ is C₁-C₄alkyl;

R₂ is halogen, C₁-C₄haloalkylsulfanyl, C₁-C₄haloalkylsulfinyl, C₁-C₄haloalkylsulfonyl, C₁-C₄haloalkoxy, C₁-C₄haloalkyl or is C₃-C₆cycloalkyl which can be mono- or polysubstituted by cyano; and

X and A are as defined under formula I above.

### Embodiment (A5)

Further preferred are compounds of formula I-1a

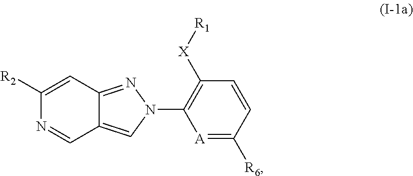

wherein R₆ is as defined as J under Embodiment (A3) above;

R₁ is C₁-C₄alkyl;

R₂ is C₁-C₄haloalkyl or is C₃-C₆cycloalkyl which can be mono- or polysubstituted by cyano; and

X and A are as defined under formula I above.

### Embodiment (A6)

Further preferred are compounds of formula I-1a

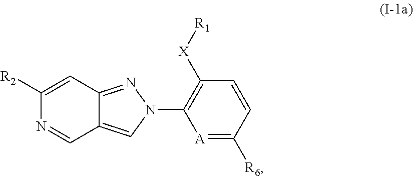

wherein R₆ is as defined as J under Embodiment (A3) above;

R₁ is C₁-C₄alkyl;

R₂ is C₁-C₄haloalkyl; and

X and A are as defined under formula I above.

### Embodiment (A7)

Further preferred are compounds of formula I-1a

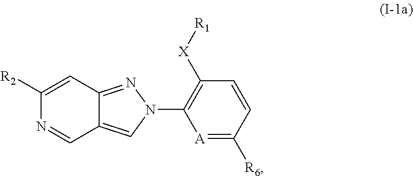

wherein R₆ is as defined as J under Embodiment (A3) above and wherein each of the groups J1 to J10 and J12 independently can be mono- or di-substituted by substituents independently selected of halogen, cyano, C₁-C₄alkyl, C₁-C₄haloalkyl, C₁-C₄haloalkoxy, C₁-C₄alkoxy, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl and C₁-C₄alkylsulfonyl; and J11 can be substituted by cyano;

R₁ is C₁-C₄alkyl;

R₂ is C₁-C₄haloalkyl; and

X and A are as defined under formula I above.

### Embodiment (A8)

Further preferred are compounds of formula I-1a

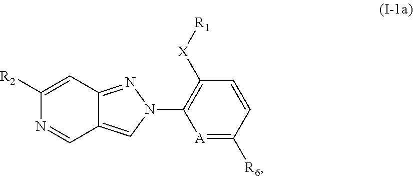

wherein R₆ is J1, J2, J10 or J11 as defined under Embodiment (A3) above and wherein each of the groups J1, J2 and J10 independently can be substituted by substituents independently selected from the group consisting of halogen, cyano, C₁-C₄haloalkyl, C₁-C₄haloalkoxy and C₁-C₄alkoxy; and J11 can be substituted by cyano;

R₁ is C₁-C₄alkyl;

R₂ is C₁-C₄haloalkyl; and

X and A are as defined under formula I above.

### Embodiment (A9)

Further preferred are compounds of formula I-1a

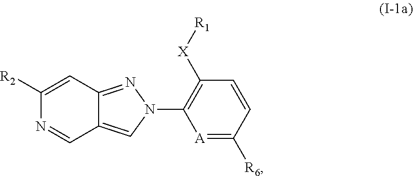

wherein R₆ is J1, J6, J7, J10 or J12 as defined under Embodiment (A3) above, wherein each of the groups J independently can be mono- or di-substituted by substituents independently selected from the group consisting of halogen, cyano, C₁-C₄alkylsulfanyl and C₁-C₄alkylsulfonyl; or

R₆ is amino; or

R₆ is NHOH; or

R₆ is NR₇R₈, wherein R₇ is C₁-C₄alkyl and R₈ is hydrogen or C₁-C₄alkyl;

R₁ is C₁-C₄alkyl;

R₂ is C₁-C₄haloalkyl; and

X and A are as defined under formula I above.

### Embodiment (A10)

Further preferred are compounds of formula I-1b

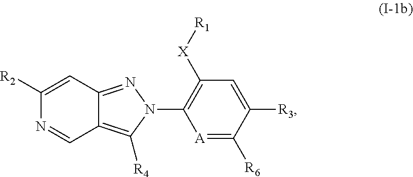

wherein

R₁ is C₁-C₄alkyl, preferably ethyl;

R₂ is C₁-C₄haloalkyl, preferably trifluoromethyl;

R₈ is hydrogen or halogen, preferably hydrogen or chlorine;

R₄ is hydrogen, halogen or C₁-C₄alkyl, preferably hydrogen, chlorine or methyl; and

X, R₆ and A are as defined under formula I above.

### Embodiment (A11)

Further preferred are compounds of formula I-1b

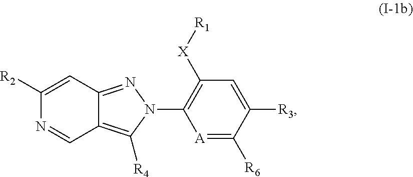

wherein

R₁ is C₁-C₄alkyl, preferably ethyl;

R₂ is C₁-C₄haloalkyl, preferably trifluoromethyl;

R₃ is hydrogen or halogen, preferably hydrogen or chlorine;

R₄ is hydrogen, halogen or C₁-C₄alkyl, preferably hydrogen, chlorine or methyl;

X is S or SO₂, preferably SO₂;

A is N or CH, preferably N; and

R₆ is selected from the group J consisting of

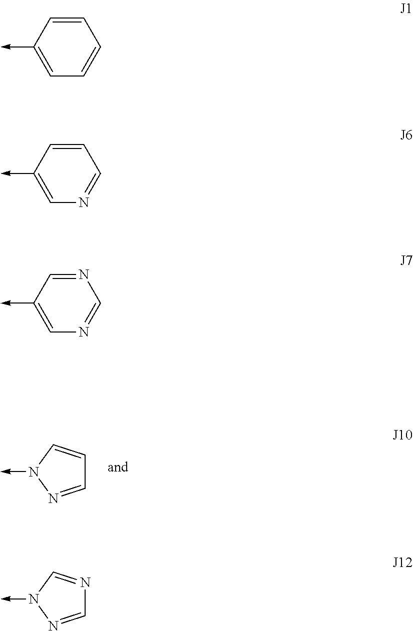

wherein each of the groups J independently can be mono- or di-substituted by substituents independently selected from the group consisting of halogen, cyano, C₁-C₄alkylsulfanyl and C₁-C₄alkylsulfonyl; or

R₆ is amino; or

R₆ is NHOH; or

R₆ is NR₇R₈, wherein R₇ is C₁-C₄alkyl, preferably methyl, and R₈ is hydrogen or C₁-C₄alkyl, preferably hydrogen or methyl; or

R₆ is C₁-C₆alkoxy, preferably methoxy or ethoxy.

### Embodiment (A12)

Further preferred are compounds of formula I-1b

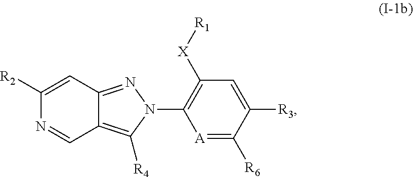

wherein

R₁ is C₁-C₄alkyl, preferably ethyl;

R₂ is C₁-C₄haloalkyl, preferably trifluoromethyl;

R₃ is hydrogen or halogen, preferably hydrogen or chlorine;

R₄ is hydrogen, halogen or C₁-C₄alkyl, preferably hydrogen, chlorine or methyl;

X is SO₂;

A is N; and

R₆ is selected from the group J consisting of

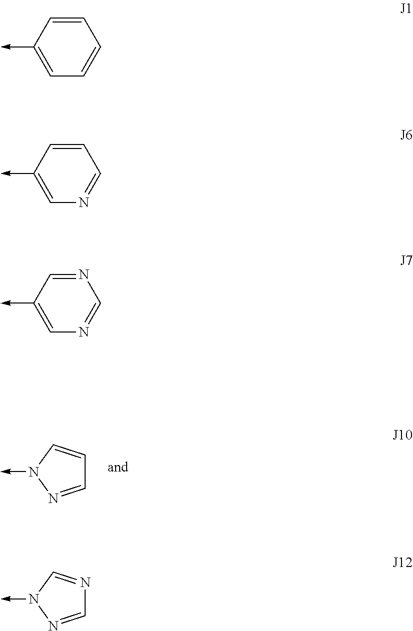

wherein each of the groups J independently can be mono- or di-substituted by substituents independently selected from the group consisting of halogen, cyano, C₁-C₄alkylsulfanyl and C₄alkylsulfonyl; or

R₆ is amino; or

R₆ is NHOH; or

R₆ is NR₇R₈, wherein R₇ is C₁-C₄alkyl, preferably methyl, and R₈ is hydrogen or C₁-C₄alkyl, preferably hydrogen or methyl; or

R₆ is C₁-C₆alkoxy, preferably methoxy or ethoxy.

In all of the preferred embodiments of formulae I-1 b above, R₆ is preferably phenyl which is mono- or di-substituted by halogen, in particular mono- or disubstituted by fluoro; or R₆ is preferably pyrimidinyl; or R₆ is preferably pyridinyl; or R₆ is preferably N-linked pyrazolyl, which may be mono-substituted by cyano; or R₆ is preferably N-linked triazolyl, which may be mono-substituted by halogen, in particular chlorine, C₁-C₄alkylsulfanyl, in particular methylsulfanyl, or C₁-C₄alkylsulfonyl, in particular methylsulfonyl; or R₆ is preferably amino, hydroxyl-amino, methyl-amino, dimethyl-amino, methoxy or ethoxy.

Another preferred group of compounds of formula I is represented by the compounds of formula I-2

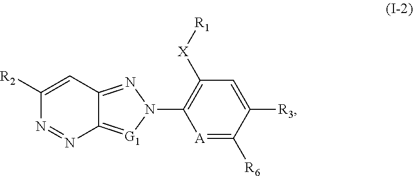

wherein the substituents X, R₁, R₂, R₃, R₆, G₁ and A are as defined under formula I above.

Further preferred embodiments of the invention are:

### Embodiment (B1)

Preferred are compounds of formula I-2, wherein

R₁ is C₁-C₄alkyl, C₃-C₆cycloalkyl-C₁-C₄alkyl or C₃-C₆cycloalkyl;

R₂ is halogen, C₁-C₄haloalkylsulfanyl, C₁-C₄haloalkylsulfinyl, C₁-C₄haloalkylsulfonyl, C₁-C₄haloalkoxy, C₁-C₄haloalkyl, cyano or is C₃-C₆cycloalkyl which can be mono- or polysubstituted by substituents independently selected from the group consisting of halogen, C₁-C₄ haloalkyl, cyano and C₁-C₄alkyl;

and

X, R₃, R₆, G₁ and A are as defined under formula I above.

### Embodiment (B2)

Further preferred are compounds of formula I-2a

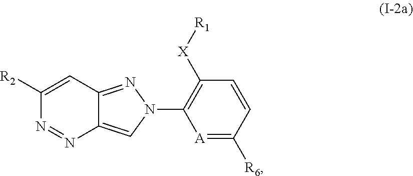

wherein

R₁ is C₁-C₄alkyl, C₃-C₆cycloalkyl-C₁-C₄alkyl or C₃-C₆cycloalkyl;

R₂ is halogen, C₁-C₄haloalkylsulfanyl, C₁-C₄haloalkylsulfinyl, C₁-C₄haloalkylsulfonyl, C₁-C₄haloalkoxy, C₁-C₄haloalkyl, cyano or is C₃-C₆cycloalkyl which can be mono- or polysubstituted by substituents independently selected from the group consisting of halogen, C₁-C₄ haloalkyl, cyano and C₁-C₄alkyl; and

X, R₆ and A are as defined under formula I above.

### Embodiment (B3)

Further preferred are compounds of formula I-2a

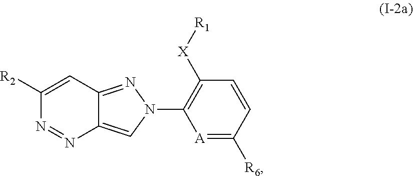

wherein

R₁ is C₁-C₄alkyl, C₃-C₆cycloalkyl-C₁-C₄alkyl or C₃-C₆cycloalkyl;

R₂ is halogen, C₁-C₄haloalkylsulfanyl, C₁-C₄haloalkylsulfinyl, C₁-C₄haloalkylsulfonyl, C₁-C₄haloalkoxy,

C₁-C₄haloalkyl, cyano or is C₃-C₆cycloalkyl which can be mono- or polysubstituted by substituents independently selected from the group consisting of halogen, C₁-C₄ haloalkyl, cyano and C₁-C₄alkyl;

X and A are as defined under formula I above; and

R₆ is selected from the group J consisting of

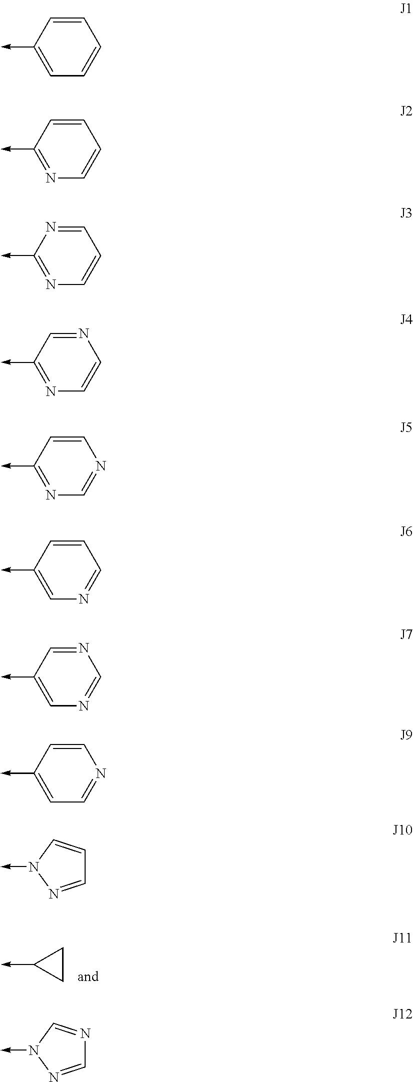

wherein each of the groups J1 to J10 and J12 independently can be mono- or di-substituted by substituents independently selected from the group consisting of halogen, cyano, nitro, C₂-C₆alkenyl, C₂-C₆haloalkenyl, C₂-C₆alkynyl, C₂-C₆haloalkynyl, C₁-C₄alkyl, C₁-C₄haloalkyl, C₁-C₄haloalkoxy, C₁-C₄alkoxy, C₁-C₄alkoxy C₁-C₄alkyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl and C₁-C₄alkylsulfonyl; and J11 can be substituted by halogen, C₁-C₄alkyl, C₁-C₄haloalkyl or cyano; or

R₆ is amino.

### Embodiment (B4)

Further preferred are compounds of formula I-2a

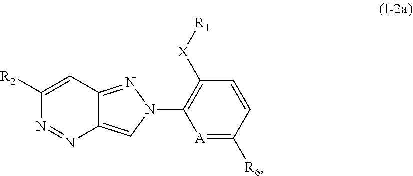

wherein R₆ is as defined as J under Embodiment (B3) above;

R₁ is C₁-C₄alkyl;

R₂ is halogen, C₁-C₄haloalkylsulfanyl, C₁-C₄haloalkylsulfinyl, C₁-C₄haloalkylsulfonyl, C₁-C₄haloalkoxy, C₁-C₄haloalkyl or is C₃-C₆cycloalkyl which can be mono- or polysubstituted by cyano; and

X and A are as defined under formula I above.

### Embodiment (B5)

Further preferred are compounds of formula I-2a

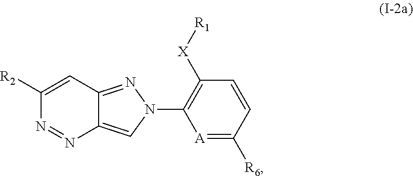

wherein R₆ is as defined as J under Embodiment (B3) above;

R₁ is C₁-C₄alkyl;

R₂ is C₁-C₄haloalkyl or is C₃-C₆cycloalkyl which can be mono- or polysubstituted by cyano; and

X and A are as defined under formula I above.

### Embodiment (B6)

Further preferred are compounds of formula I-2a

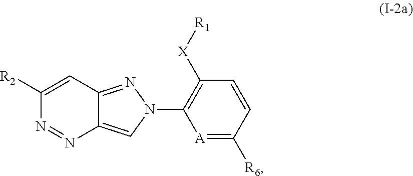

wherein R₆ is as defined as J under Embodiment (B3) above;

R₁ is C₁-C₄alkyl;

R₂ is C₁-C₄haloalkyl; and

X and A are as defined under formula I above.

### Embodiment (B7)

Further preferred are compounds of formula I-2a

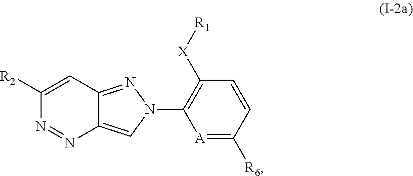

wherein R₆ is as defined as J under Embodiment (A3) above and wherein each of the groups J1 to J10 and J12 independently can be mono- or di-substituted by substituents independently selected of halogen, cyano, C₁-C₄alkyl, C₁-C₄haloalkyl, C₁-C₄haloalkoxy, C₁-C₄alkoxy, C₁-C₄haloalkylsulfanyl, C₄haloalkylsulfinyl, C₁-C₄haloalkylsulfonyl, C₁-C₄alkylsulfanyl, C₁-C₄alkylsulfinyl and C₁-C₄alkylsulfonyl;

and J11 can be substituted by cyano;

R₁ is C₁-C₄alkyl;

R₂ is C₁-C₄haloalkyl; and

X and A are as defined under formula I above.

### Embodiment (B8)

Further preferred are compounds of formula I-2a

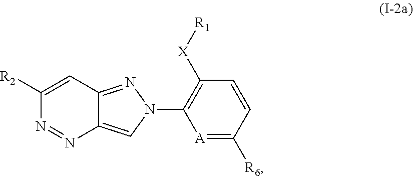

wherein R₆ is J1, J2, J10 or J11 as defined under Embodiment (A3) above and wherein each of the groups J1, J2 and J10 independently can be substituted by substituents independently selected from the group consisting of halogen, cyano, C₁-C₄haloalkyl, C₁-C₄haloalkoxy and C₁-C₄alkoxy; and J11 can be substituted by cyano;

R₁ is C₁-C₄alkyl;

R₂ is C₁-C₄haloalkyl; and

X and A are as defined under formula I above.

### Embodiment (B9)

Further preferred are compounds of formula I-2a

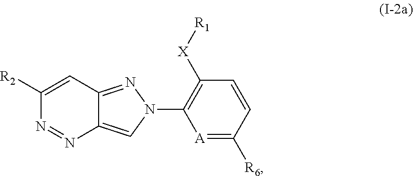

wherein R₆ is J12 as defined under Embodiment (A3) above, which can be mono-substituted by halogen;

R₁ is C₁-C₄alkyl;

R₂ is C₁-C₄haloalkyl; and

X and A are as defined under formula I above.

### Embodiment (B10)

Further preferred are compounds of formula I-2b

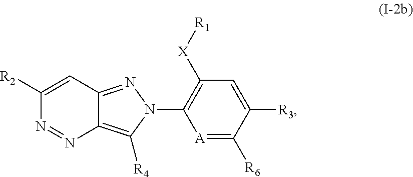

wherein

R₁ is C₁-C₄alkyl, preferably ethyl;

R₂ is C₁-C₄haloalkyl, preferably trifluoromethyl;

R₃ is hydrogen or halogen, preferably hydrogen or chlorine;

R₄ is hydrogen, halogen or C₁-C₄alkyl, preferably hydrogen, chlorine or methyl; and

X, R₆ and A are as defined under formula I above.

### Embodiment (B11)

Further preferred are compounds of formula I-2b

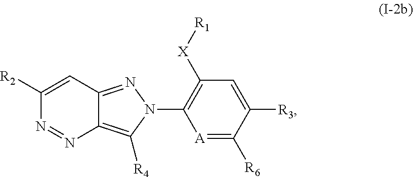

wherein

R₁ is C₁-C₄alkyl, preferably ethyl;

R₂ is C₁-C₄haloalkyl, preferably trifluoromethyl;

R₃ is hydrogen or halogen, preferably hydrogen or chlorine;

R₄ is hydrogen, halogen or C₁-C₄alkyl, preferably hydrogen, chlorine or methyl;

X is S or SO₂, preferably SO₂;

A is N or CH, preferably N; and

R₆ is selected from the group J consisting of

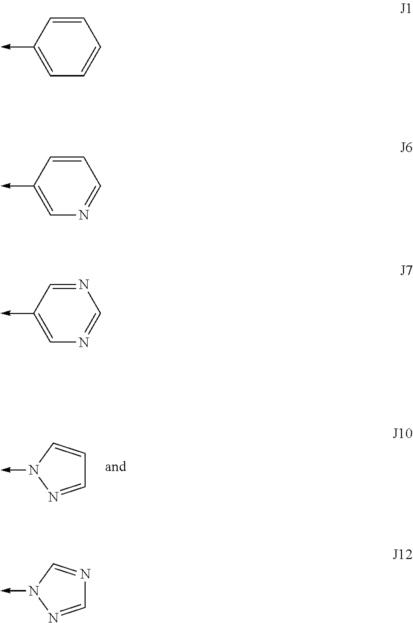

wherein each of the groups J independently can be mono- or di-substituted by substituents independently selected from the group consisting of halogen, cyano, C₁-C₄alkylsulfanyl and C₄alkylsulfonyl; or

R₆ is amino; or

R₆ is NHOH; or

R₆ is NR₇R₈, wherein R₇ is C₁-C₄alkyl, preferably methyl, and R₈ is hydrogen or C₁-C₄alkyl, preferably hydrogen or methyl; or

R₆ is C₁-C₆alkoxy, preferably methoxy or ethoxy.

In all of the preferred embodiments of formulae I-1, I-1a, I-2 and I-2a above, X is preferably S or SO₂.

In all of the preferred embodiments of formulae I-1, I-1a, I-2 and I-2a above, R₆ is preferably phenyl which is mono- or di-substituted by halogen, in particular disubstituted by fluoro.

An especially preferred embodiment of the invention comprises compounds of formula I represented by the compounds of formula I-3

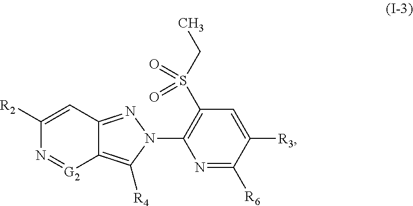

wherein

R₂ is C₁-C₄haloalkyl, preferably trifluoromethyl;

R₃ is hydrogen or halogen, preferably hydrogen or chlorine;

R₄ is hydrogen, halogen or C₁-C₄alkyl, preferably hydrogen, chlorine or methyl;

G₂ is CH or N; and

R₆ is selected from the group J consisting of

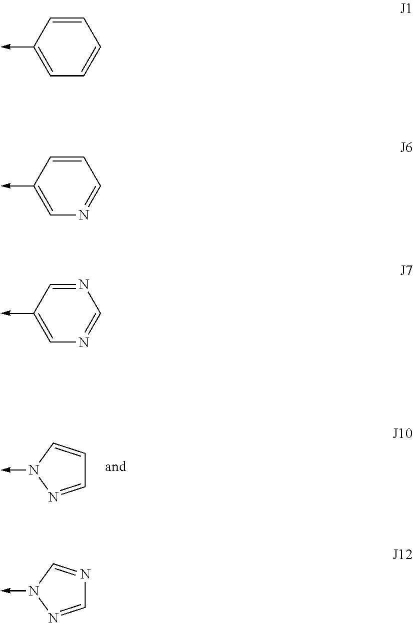

wherein each of the groups J independently can be mono- or di-substituted by substituents independently selected from the group consisting of halogen, cyano, C₁-C₄alkylsulfanyl and C₁-C₄alkylsulfonyl; or

R₆ is amino; or

R₆ is NHOH; or

R₆ is NR₇R₈, wherein R₇ is C₁-C₄alkyl, preferably methyl, and R₈ is hydrogen or C₁-C₄alkyl, preferably hydrogen or methyl; or

R₆ is C₁-C₆alkoxy, preferably methoxy or ethoxy;

and agrochemically acceptable salts, stereoisomers, enantiomers, tautomers and N-oxides of the compounds of formula I-3.

In the especially preferred embodiment of formula I-3 above, R₆ is preferably phenyl which is mono- or di-substituted by halogen, in particular mono- or disubstituted by fluoro; or R₆ is preferably pyrimidinyl; or R₆ is preferably pyridinyl; or R₆ is preferably N-linked pyrazolyl, which may be mono-substituted by cyano; or R₆ is preferably N-linked triazolyl, which may be mono-substituted by halogen, in particular chlorine, C₁-C₄alkylsulfanyl, in particular methylsulfanyl, or C₁-C₄alkylsulfonyl, in particular methylsulfonyl; or R₆ is preferably amino, hydroxyl-amino, methyl-amino, dimethyl-amino, methoxy or ethoxy.

The process according to the invention for preparing compounds of formula (I) is carried out by methods known to those skilled in the art, or in analogy to processes described in the literature, for example, in WO 2013/191113 using the appropriate starting materials.

More specifically, the subgroup of compounds of formula I, wherein X is SO (sulfoxide) and/or SO₂ (sulfone), may be obtained by means of an oxidation reaction of the corresponding sulfide compounds of formula I, wherein X is S, involving reagents such as, for example, m-chloroperoxybenzoic acid (mCPBA), hydrogen peroxide, oxone, sodium periodate, sodium hypochlorite or tert-butyl hypochlorite amongst other oxidants. The oxidation reaction is generally conducted in the presence of a solvent. Examples of the solvent to be used in the reaction include aliphatic halogenated hydrocarbons such as dichloromethane and chloroform; alcohols such as methanol and ethanol; acetic acid; water; and mixtures thereof. The amount of the oxidant to be used in the reaction is generally 1 to 3 moles, preferably 1 to 1.2 moles, relative to 1 mole of the sulfide compounds I to produce the sulfoxide compounds I, and preferably 2 to 2.2 moles of oxidant, relative to 1 mole of the sulfide compounds I to produce the sulfone compounds I. Such oxidation reactions are disclosed, for example, in WO 2013/018928.

Indazoles, aza-indazoles and/or diaza-indazoles, may be made using processes that are well known and have been described for example in WO 2013/191113; Synlett (2013), 24(12), 1573-1577; Journal of the Chemical Society, Chemical Communications (1991), (20), 1466-7; Organic Letters (2014), 16(11), 3114-3117; or for a review on more general synthesis for this type of derivatives, see for example Science of Synthesis (2002), 12, 227-324 and European Journal of Organic Chemistry (2008), (24), 4073-4095. All of these process could be use to access indazoles derivatives. One possible process is summarized in scheme 1 for compounds of formula I:

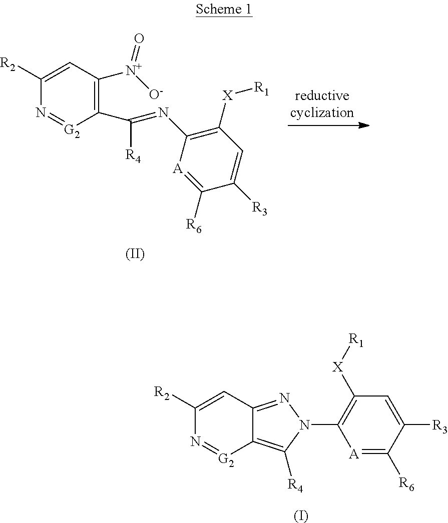

Compounds of formula (I) may be prepared by reaction of a compound of formula (II) under reductive cyclisation conditions using a reducing agent, such as trialkyl phosphite (more specifically, for example triethyl phosphite), trialkylphosphine or triphenylphosphine. The principle of this reductive cyclisation is analogous to the known Cadogan reaction. Alternatively, this reaction may be conducted in presence of a metal catalyst, for example a molybdenum(VI) catalyst such as MoO₂Cl₂(dmf)₂ [molybdenyl chloride-bis(dimethylformamide)], or more generally with transition metal complexes in combination with a reducing agent such as triethylphosphite, triphenylphosphine or CO. Suitable solvents may include use of excess of the reducing agent (such as triethyl phosphite), or for example toluene or xylene at temperatures between room temperature and 200° C., preferably between 50 and 160° C., optionally under microwave conditions.

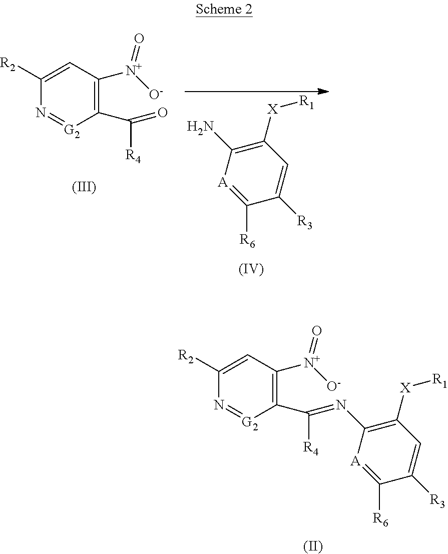

Compounds of formula (II) may be prepared (scheme 2) by reaction of aldehyde or ketone derivatives of formula (III) with amine derivatives of formula (IV), usually upon heating and optionally under microwave conditions. The formation of compounds of formula (II) may require water removal, either by azeotropical distillation, or with a drying agent such as for example TiCl₄ or molecular sieves. The formation of the Schiff bases of formula (II) is very well known to those skilled in the art, and methods are well described in the literature, see for example, Molbank (2006), M514 or March's Advanced Organic Chemistry: Reactions, Mechanisms, and Structure, 5th Edition p 1185-1187 and cited documents therein. Suitable solvents may include for example toluene or xylene at temperatures between room temperature and 200° C., preferably between 50 and 160° C.

Compounds of formula (III) are either known, commercially available or may be made by methods known to a person skilled in the art.

Compounds of formula (IV) are either known, commercially available or may be made by methods known to a person skilled in the art.

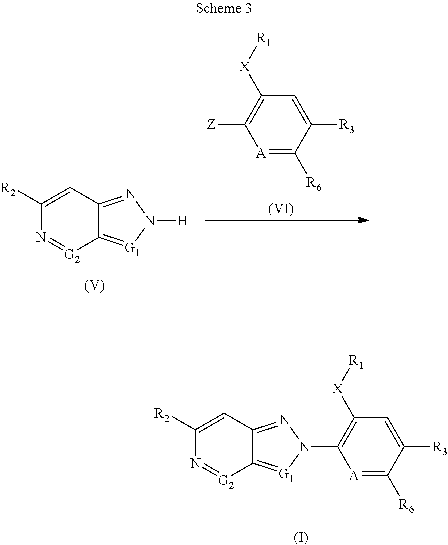

In an alternative method depicted in scheme 3, compounds of formula I can also be prepared by reacting compounds of formula V, wherein G₁, G₂ and R₂ have the values defined in formula I with a compound of formula (VI), wherein X, R₁, R₃, R₆ and A have the values defined in formula I, and wherein Z is a leaving group like, for example, fluorine, chlorine, bromine or iodine, or an aryl- or (halo)alkylsulfonate, or any other similar leaving group. For example, this reaction, called S_{N}Ar reaction (aromatic nucleophilic substitution reaction) can be done in a presence of base such as for example sodium, potassium or lithium carbonate, or sodium hydride, in a solvent such as dimethylformamide, at temperatures between room temperature and 200° C., with or without microwave irradiation. An example of this type of reaction is described in WO 2007/113596 and Journal of Medicinal Chemistry, 52(22), 7170-7185, 2009. In an alternative method, a compound of formula (VI) wherein Z is chlorine, bromine or iodine, or any other appropriate leaving group, could be coupled with compounds of formula V by using metal catalyst coupling conditions such as copper catalyst or palladium catalyst, for example using copper(I) iodide as copper catalyst, with or without an additive such as L-proline or N,N′-dimethylethylenediamine, in presence of a base such as, for example potassium carbonate. Said alternative method is for example described in WO 2006/107771 and WO 2012/083105.

Compounds of formula (V) are either known, commercially available or may be made by methods known to a person skilled in the art.

Compounds of formula (VI) are either known, commercially available or may be made by methods known to a person skilled in the art. Particular examples wherein compounds of formula (VI) are defined as compounds of formula (VIa) and (VIb) are described in scheme 4 and scheme 5.

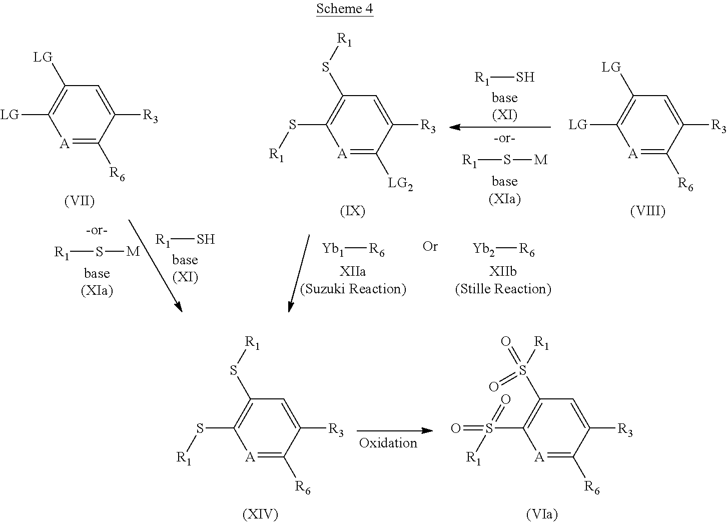

Compounds of formula (VIa), wherein R₃, R₆, R₁ and A have the values defined in formula I, may be prepared (scheme 4) by oxidation of compounds of formula (XIV). The reaction can be performed with reagents like, for example a peracid as peracetic acid or m-chloroperbenzoic acid, or a hydroperoxide as for example hydrogen peroxide or tert-butylhydroperoxide, or an inorganic oxidant, like a mono-peroxodisulfate salt or potassium permanganate, preferentially meta-chloroperbenzoic acid.

Compounds of formula (XIV) wherein R₃, R₆, R₁ and A have the values defined in formula I, may be prepared (scheme 4) by substitution of the two leaving groups (LG) of compounds of formula (VII), wherein LG is, for example chlorine or fluorine, by reaction with compounds of formula XI

R₁—SH (XI),

or a salt thereof, wherein R₁ is as defined in formula I, optionally in the presence of a suitable base, such as alkali metal carbonates, for example sodium carbonate and potassium carbonate, or alkali metal hydrides such as sodium hydride, or alkali metal hydroxides such as sodium hydroxide and potassium hydroxide, in an inert solvent at temperatures preferably between 25-120° C. Examples of solvent to be used include ethers such as THF, ethylene glycol dimethyl ether, tert-butylmethyl ether, and 1,4-dioxane, aromatic hydrocarbons such as toluene and xylene, nitriles such as acetonitrile or polar aprotic solvents such as N,N-dimethylformamide, N,N-dimethylacetamide, N-methyl-2-pyrrolidone or dimethyl sulfoxide. Examples of salts of the compound of formula XI include compounds of the formula XIa

R₁—S—M (XIa),

wherein R₁ is as defined above and wherein M is, for example, sodium or potassium.

Under similar conditions, compounds of formula (IX), wherein R₃, R₁ and A have the values defined in formula I, and wherein LG₂ is bromine or iodine, may be prepare from compounds of formula (VIII), wherein R₃ and A have the values defined in formula I, and wherein LG is, for example chlorine or fluorine, and LG₂ is bromine or iodine, by reaction with a reagent XI or XIa. The transformation of compounds of formula (IX), wherein R₃, R₁ and A have the values defined in formula I, and wherein LG₂ is bromine or iodine, to compounds of formula (XIV), wherein R₆, R₃, R₁ and A have the values defined in formula I, via transformation of LG₂ to R₆ can be perform by methods well known to a person skilled in the art. For example, compounds of formula (XIV) wherein R₁, R₃ and A have the values defined in formula I and R₆ is, for example, cyclopropane, aryl or heteroaryl can be prepared by a Stille reaction of compounds of formula XIIb wherein Y_{b2} is a trialkyl tin derivative, preferably tri-n-butyl tin, with compounds of formula (IX). Such Stille reactions are usually carried out in the presence of a palladium catalyst, for example tetrakis(triphenylphosphine)palladium(O), or (1,1′bis(diphenylphosphino)-ferrocene)dichloropalladium-dichloromethane (1:1 complex), in an inert solvent such as DMF, acetonitrile, or dioxane, optionally in the presence of an additive, such as cesium fluoride, or lithium chloride, and optionally in the presence of a further catalyst, for example copper(I)iodide. Such Stille couplings are also well known to those skilled in the art, and have been described in for example J. Org. Chem., 2005, 70, 8601-8604, J. Org. Chem., 2009, 74, 5599-5602, and Angew. Chem. Int. Ed., 2004, 43, 1132-1136. Alternatively, compounds of formula (XIV), wherein

R₃, R₁ and A have the values defined in formula I and R₆ is, for example, cyclopropane, aryl or heteroaryl can also be prepared by a Suzuki reaction, which involves reacting compounds of formula (IX), wherein LG₂ is a leaving group, for example, chlorine, bromine or iodine with compounds of formula XIIa, wherein Y_{b1} can be a boron-derived functional group, as for example B(OH)₂ or B(OR_{b1})₂ wherein R_{b1} can be a C₁-C₄alkyl group or the two groups OR_{b1} can form together with the boron atom a five membered ring, as for example a pinacol boronic ester. The reaction can be catalyzed by a palladium based catalyst, for example tetrakis(triphenylphosphine)-palladium(O) or (1,1′bis(diphenylphosphino)-ferrocene)dichloropalladium-dichloromethane (1:1 complex), in presence of a base, like sodium carbonate or cesium fluoride, in a solvent or a solvent mixture, like, for example a mixture of 1,2-dimethoxyethane and water, or of dioxane and water, preferably under an inert atmosphere. The reaction temperature can preferentially range from room temperature to the boiling point of the reaction mixture. Such Suzuki reactions are well known to those skilled in the art and have been reviewed, for example J. Orgmet. Chem. 576, 1999, 147-168.

Compounds of formula (VII) and compounds of formula (VIII) are either known, commercially available or may be made by methods known to a person skilled in the art.

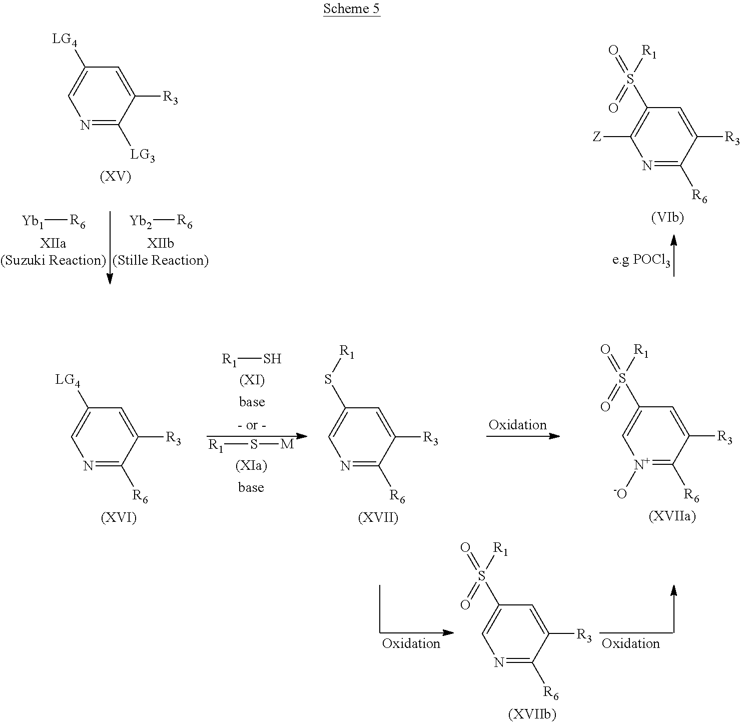

Alternatively, compounds of formula (VIb), wherein R₆, R₃ and R₁ have the values defined in formula I, and wherein Z is a leaving group like, for example, fluorine, chlorine, bromine or iodine, preferably chlorine, may be prepare from compounds of formula (XVIIa), wherein R₁, R₃ and R₆ are as described for compounds of formula I above, via rearrangement, for example mediated by an halogenating agent such as phosphoroxychlorid POCl₃, neat or in the appropriate solvent, such as chloroforme or toluene and by temperatures from below 20° C. to up to the boiling point of the solvent system. Such rearrangement reactions are well known from the literature, for example, see Chemical & Pharmaceutical Bulletin 1988 36(6), 2244-7; ACS Catalysis 2014 4(8), 2741-2745; ACS Chemical Biology, 2013 8(11), 2501-2508; Journal of Medicinal Chemistry, 2013 56(9), 3666-3679 or Journal of Medicinal Chemistry 2011 54(5), 1511-1528.

N-oxide compounds of formula (XVIIa), wherein R₁, R₃ and R₆ are as described for compounds of formula I above, may be prepared from a compound of formula (XVII), wherein R₁, R₃ and R₆ are as described for compounds of formula I above, via oxidation by reaction with a suitable oxidizing agent, such as meta-perbenzoic acid or hydrogen peroxide in the appropriate inert solvent, such as for example dichloromethane or chloroform. The direct transformation XVII to XVIIa will require at least three equivalents of the oxidant. This reaction could also be done in two steps via 1) oxidation of compound of formula (XVII) to compounds of formula (XVIIb) (typically two equivalents of oxidant) then 2) by further oxidation of compound of formula (XVIIb) to compounds of formula XVIIa (typically one equivalent of oxidant) via the same or different types of oxidant. Such oxidations are known from the literature, for example from WO 2013/018928, WO 2010/073128, Synthetic Communications 2013, 43(8), 1092-1100 or Arkivoc 2001 (i) 242-268.

Compounds of formula (XVII), wherein R₁, R₃ and R₆ are as described for compounds of formula I above, may be prepare from compounds of formula (XVI), wherein R₃ and R₆ are as described for compounds of formula I above, and wherein LG₄ is a leaving group, like, for example, fluorine, chlorine, bromine or iodine, preferably fluorine, by reaction with compounds of formula XI or XIa (substitution of the leaving group LG₄) under the same conditions already described in scheme 4 (transformation of compound of formula (VII) to (XIV)).

Compounds of formula (XVI) are either known, commercially available or may be made by methods known to a person skilled in the art. One particular example is described in scheme 5, the introduction of R₆ via Suzuki reaction or Stille coupling under similar conditions described for the synthesis of compounds of formula (XIV) in scheme 4 (transformation of compound of formula (IX) to (XIV)), and making use of reagents XIIa or XIIb.

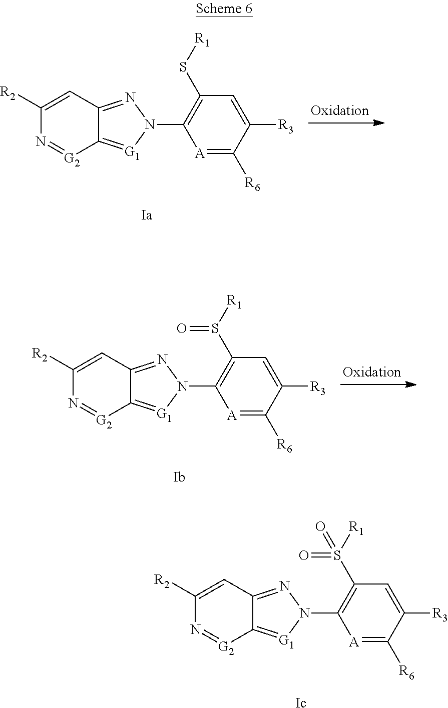

Compounds of formula Ib, wherein A, R₁, R₂, R₃, R₆, G₁ and G₂ have the values defined in formula I, can be prepared (scheme 6) by oxidation of compounds of formula Ia, wherein A, R₁, R₂, R₃, R₆, G₁ and G₂ have the values defined in formula I. The reaction can be performed with reagents like, for example a peracid as peracetic acid or m-chloroperbenzoic acid, or a hydroperoxide as for example hydrogen peroxide or tert-butylhydroperoxide, or an inorganic oxidant, like a mono-peroxodisulfate salt or potassium permanganate, preferentially meta-chloroperbenzoic acid. In a similar way, compounds of formula Ic, wherein A, R₁, R₂, R₃, R₆, G₁ and G₂ have the values defined in formula I, can be prepared by oxidation of compounds of formula Ib. These reactions can be performed in various organic or aqueous solvents compatible to these conditions, by temperatures from below 0° C. up to the boiling point of the solvent system and the number of equivalents of oxidant will determinate the degrees of oxidation of the sulphur, e.g. with two or more equivalents of oxidant, the compound of formula Ic can be prepare directly from compound of formula Ia.

Alternative approaches towards compounds of formula I, wherein X, A, R₁, R₂, R₃, R₆ and G₂are as defined in formula I above, are shown in scheme 7, taking advantage of functional group inter-conversion of substituent R₄. For example, compounds of formula I, wherein X, A, R₁, R₂, R₃, R₆ and G₂are as defined in formula I above, and in which R₄ is hydrogen, can be prepared from compounds of formula I, wherein X, A, R₁, R₂, R₃, R₆ and G₂are as defined in formula I above, and in which R₄ is halogen (compounds Id), preferably chlorine or bromine, by means of a reductive dehalogenation. Such a hydrodehalogenation can be achieved, for example, using zinc dust and acetic acid or trifluoroacetic acid, or mixtures thereof, at temperatures between 0° C. and 120° C., preferably between 50° C. and reflux temperature, as described, for example, in Journal of the Chemical Society, Perkin Transactions 1: Organic and Bio-Organic Chemistry (1972-1999), (10), 2501-6, 1983 or in US20100076027.

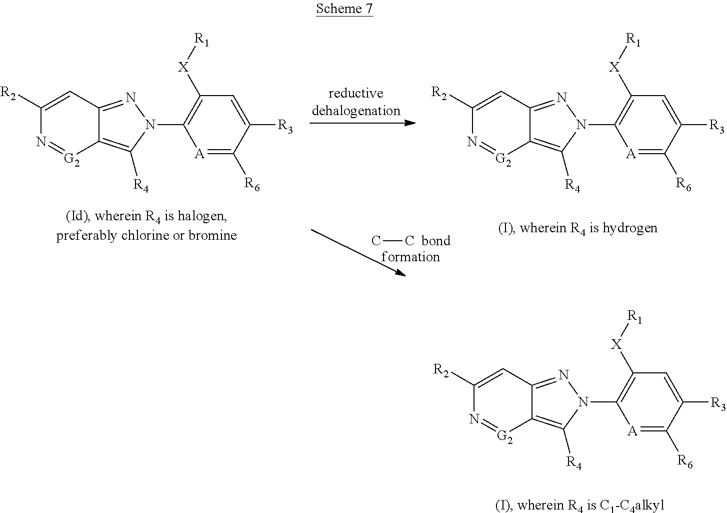

Conversely, compounds of formula I, wherein X, A, R₁, R₂, R₃, R₆ and G₂are as defined in formula I above, and in which R₄ is C₁-C₄alkyl, can be prepared from compounds of formula I, wherein X, A, R₂, R₃, R₆ and G₂are as defined in formula I above, and in which R₄ is halogen (compounds Id), preferably chlorine or bromine, by means of a C—C bond formation reaction typically under palladium-catalyzed cross-coupling conditions. Such Suzuki-Miyaura cross-coupling reactions between compounds of formula Id and C₁-C₄alkyl boronic acids or C₁-C₄alkyl boronate esters are well known to a person skilled in the art. In the particular situation where R₄ is methyl, compounds of formula Id can be reacted, for example, with trimethylboroxine (also known as 2,4,6-trimethyl-1,3,5,2,4,6-trioxatriborinane) in the presence of palladium catalyst, such as tetrakis(triphenylphosphine)-palladium(0), and a base, such as sodium or potassium carbonate, in a solvent, such as N,N-dimethylformamide, dioxane or dioxane-water mixtures, at temperatures between room temperature and 160° C., optionally under microwave heating conditions, and preferably under inert atmosphere. Such conditions are described, for example, in Tetrahedron Letters (2000), 41(32), 6237-6240.

Compounds of formula I, wherein X, A, R₁, R₂, R₃, R₆ and G₂are as defined in formula I above, and in which R₄ is halogen (compounds Id), preferably chlorine or bromine,

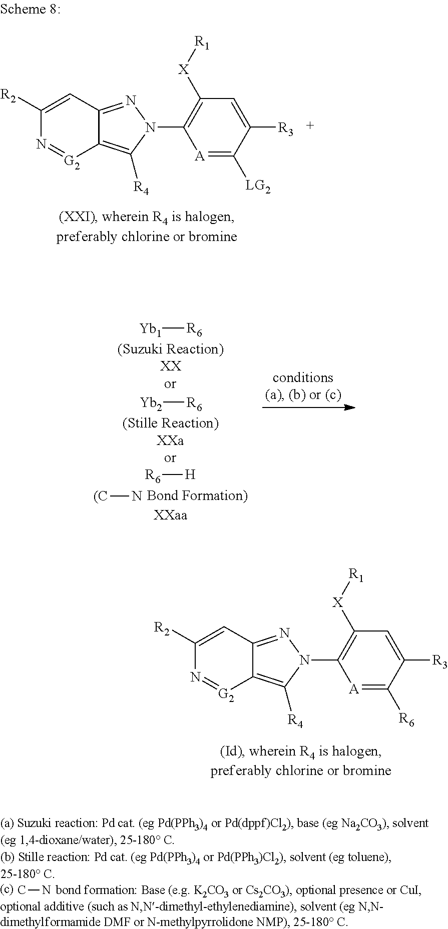

can be prepared by a Suzuki reaction (scheme 8), which involves for example, reacting compounds of formula XXI, wherein X, A, R₁, R₂, R₃ and G₂are as defined in formula I above, and wherein R₄ is halogen, preferably chlorine or bromine, and in which LG₂ is a leaving group like, for example, chlorine, bromine or iodine (preferably chlorine or bromine), or an aryl- or (halo)alkylsulfonate such as trifluoromethanesulfonate, with compounds of formula XX, wherein R₆ is as defined in formula I above, and wherein Y_{b1} can be a boron-derived functional group, such as for example B(OH)₂ or B(OR_{b1})₂ wherein R_{b1} can be a C₁-C₄alkyl group or the two groups OR_{b1} can form together with the boron atom a five membered ring, as for example a pinacol boronic ester. The reaction may be catalyzed by a palladium based catalyst, for example tetrakis(triphenylphosphine)palladium(O), tris(dibenzylidene-acetone)dipalladium(O) in presence of a phosphine ligand, such as tricyclohexylphosphane, (1,1′bis(diphenyl-phosphino)ferrocene)dichloro-palladium-dichloromethane (1:1 complex) or chloro(2-dicyclohexylphosphino-2′,4′,6′-triisopropyl-1,1′-biphenyl)[2-(2′-amino-1,1′-biphenyl)]palladium(II) (XPhos palladacycle), in presence of a base, like sodium carbonate, tripotassium phosphate or cesium fluoride, in a solvent or a solvent mixture, like, for example dioxane, acetonitrile, N,N-dimethylformamide, a mixture of 1,2-dimethoxyethane and water or of dioxane/water, or of toluene/water, preferably under inert atmosphere. The reaction temperature can preferentially range from room temperature to the boiling point of the reaction mixture, or the reaction may be performed under microwave irradiation. Such Suzuki reactions are well known to those skilled in the art and have been reviewed, for example, in J. Orgmet. Chem. 576, 1999, 147-168. This method is particularly suitable when R₆ is cyclopropyl, phenyl, pyridinyl or pyrimidinyl.

Alternatively compounds of formula (Id) may be prepared by a Stille reaction between compounds of formula XXa, wherein R₆ is as defined in formula I above, and wherein Y_{b2} is a trialkyl tin derivative, preferably tri-n-butyl tin or tri-methyl-tin, and compounds of formula XXI, wherein X, A, R₁, R₂, R₃ and G₂are as defined in formula I above, and wherein R₄ is halogen, preferably chlorine or bromine, and in which LG₂ is a leaving group like, for example, chlorine, bromine or iodine (preferably chlorine or bromine), or an aryl- or (halo)alkylsulfonate such as trifluoromethanesulfonate. Such Stille reactions are usually carried out in the presence of a palladium catalyst, for example tetrakis(triphenylphosphine) palladium(O), or bis(triphenylphosphine) palladium(II) dichloride, in an inert solvent such as N,N-di-methylformamide, acetonitrile, toluene or dioxane, optionally in the presence of an additive, such as cesium fluoride, or lithium chloride, and optionally in the presence of a further catalyst, for example copper(I)iodide. Such Stille couplings are also well known to those skilled in the art, and have been described in for example J. Org. Chem., 2005, 70, 8601-8604, J. Org. Chem., 2009, 74, 5599-5602, and Angew. Chem. Int. Ed., 2004, 43, 1132-1136.

When R₆ is a five- to six-membered, aromatic, partially saturated or fully saturated ring system linked via a nitrogen ring atom to the ring which contains the substituent X—R₁, then compounds of formula (Id), can be prepared from compounds of formula XXI, wherein X, A, R₁, R₂, R₃ and G₂are as defined in formula I above, and wherein R₄ is halogen, preferably chlorine or bromine, and in which LG₂ is a leaving group like, for example, chlorine, bromine or iodine (preferably chlorine or bromine), or an aryl- or (halo)alkylsulfonate such as trifluoromethanesulfonate, by reaction with a heterocycle R₆—H (which contains an appropriate NH functionality) XXaa, wherein R₆ is as defined above, in the presence of a base, such as potassium carbonate K₂CO₃ or cesium carbonate Cs₂CO₃, optionally in the presence of a copper catalyst, for example copper(I) iodide, with or without an additive such as L-proline, N,N′-dimethylcyclohexane-1,2-diamine or N,N′-dimethylethylene-diamine, in an inert solvent such as N-methylpyrrolidone NMP or N,N-dimethylformamide DMF at temperatures between 20-150° C., optionally under microwave irradiation. Such a reaction (C—N Bond Formation) is illustrated below (scheme 9) for the heterocycle R₆—H J10, wherein J10 is as defined above, wherein J10 may be optionally substituted, in particular J10 can be mono- or di-substituted by substituents independently selected from the group consisting of halogen, cyano, C₁-C₄alkylsulfanyl and C₁-C₄alkylsulfonyl,

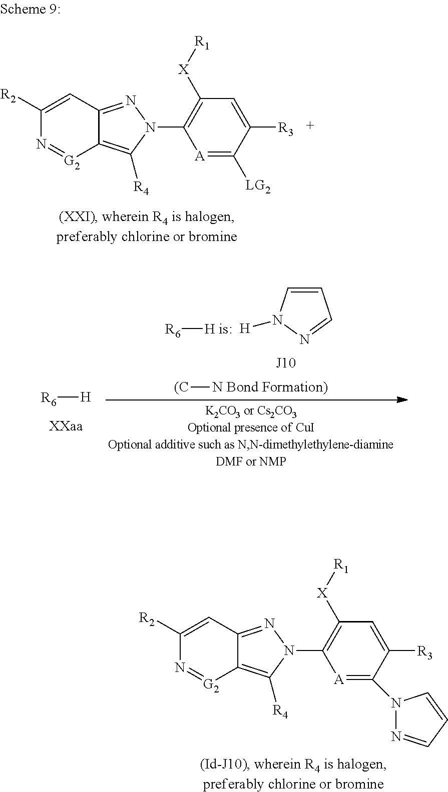

to give compounds of formula (Id-J10), a particular sub-group of compounds of formula (Id), wherein X, A, R₁, R₂, R₃ and G₂are as previously defined.

A large number of compounds of the formula XX, XXa and XXaa are known, commercially available or can be prepared by those skilled in the art.

In the particular situation within scheme 8 when R₆ is an optionally substituted triazole linked via a nitrogen atom to the ring which contains the substituent X—R₁, then compounds of formula (Id) can be prepared from compounds of formula XXI, wherein X, A, R₁, R₂, R₃ and G₂are as defined in formula I above, and wherein R₄ is halogen, preferably chlorine or bromine, and in which LG₂ is a leaving group like, for example, chlorine, bromine or iodine (preferably chlorine or bromine), or an aryl- or alkyl-sulfonate such as trifluoromethanesulfonate, by reaction with an optionally substituted triazole R₆—H (which contains an appropriate NH functionality) XXaa, wherein R₆ is N-linked triazolyl, in solvents such as alcohols (eg. methanol, ethanol, isopropanol, or higher boiling linear or branched alcohols), N,N-dimethylformamide DMF, pyridine or acetic acid, optionally in the presence of an additional base, such as potassium carbonate K₂CO₃ or cesium carbonate Cs₂CO₃, optionally in the presence of a copper catalyst, for example copper(I) iodide, at temperatures between 20-180° C., optionally under microwave irradiation (analogy to scheme 9, wherein R₆—H is replaced with J12).

Compounds of formula XXI, wherein X, A, R₁, R₂, R₃ and G₂are as defined in formula I above, and wherein R₄ is halogen, preferably chlorine or bromine, and in which LG₂ is a leaving group like, for example, chlorine, bromine or iodine (preferably chlorine or bromine), or an aryl- or (halo)alkylsulfonate such as trifluoromethanesulfonate,

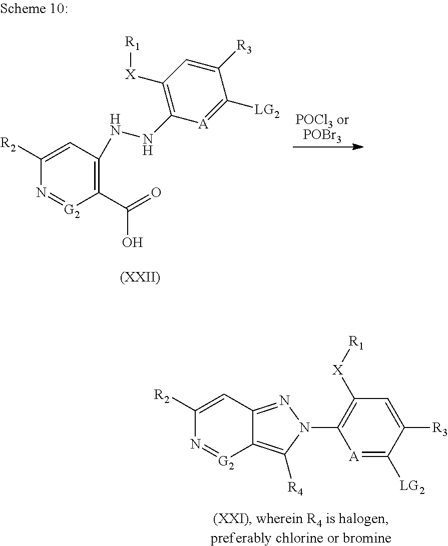

can be prepared (scheme 10) by treating compounds of formula XXII, wherein X, A, R₁, R₂, R₃ and G₂ are as defined in formula I above, and in which LG₂ is a leaving group like, for example, chlorine, bromine or iodine (preferably chlorine or bromine), or an aryl- or (halo)alkylsulfonate such as trifluoromethanesulfonate, with reagents such as phosphorus oxychloride or phosphorus oxybromide, at temperatures ranging preferentially from room temperature to the boiling point of the reaction mixture, optionally under microwave irradiation.

Compounds of formula XXII, wherein X, A, R₁, R₂, R₃ and G₂are as defined in formula I above, and in which LG₂ is a leaving group like, for example, chlorine, bromine or iodine (preferably chlorine or bromine), or an aryl- or (halo)alkylsulfonate such as trifluoromethanesulfonate,

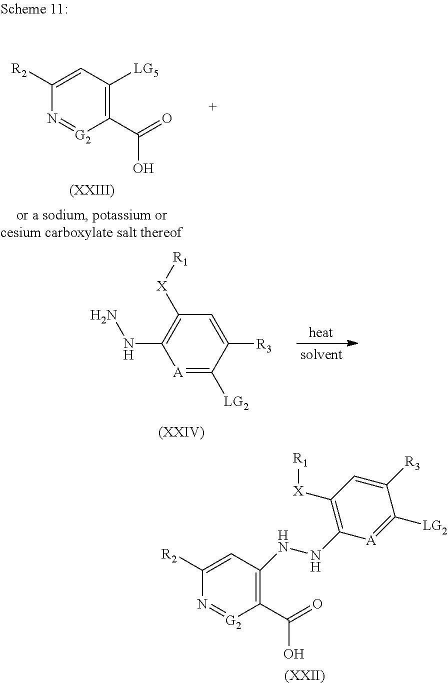

can be prepared (scheme 11) by reacting compounds of formula XXIII, or a salt thereof (such as a sodium, potassium or cesium carboxylate salt), wherein R₂ and Glare as defined in formula I above, and in which LG₅ is a halogen leaving group like, for example, chlorine, bromine or iodine (preferably chlorine or iodine), with compounds of formula XXIV, wherein X, A, R₁ and R₃are as defined in formula I above, and in which LG₂ is a leaving group like, for example, chlorine, bromine or iodine (preferably chlorine or bromine), or an aryl- or (halo)alkylsulfonate such as trifluoromethanesulfonate. This transformation may be conducted in solvents such as alcohols (eg. methanol, ethanol, isopropanol, pentanol, or higher boiling linear or branched alcohols), N,N-dimethylformamide, pyridine or acetonitrile, optionally in the presence of an additional base, such as potassium carbonate K₂CO₃ or cesium carbonate Cs₂CO₃, optionally in the presence of a copper catalyst, for example copper(I) iodide, at temperatures between 20-200° C., preferably at temperatures ranging from room temperature to the boiling point of the reaction mixture, optionally under microwave irradiation.

Compounds of formula XXIV, or a salt thereof, wherein X, A, R₁ and R₃are as defined in formula I above, and in which LG₂ is a leaving group like, for example, chlorine, bromine or iodine (preferably chlorine or bromine), or an aryl- or (halo)alkylsulfonate such as trifluoromethanesulfonate,

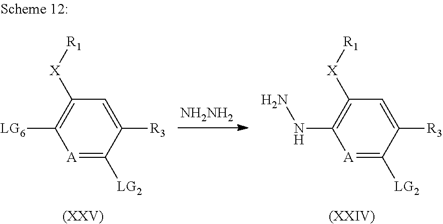

can be prepared (scheme 12) by the reaction of hydrazine (or a salt thereof), possibly in form of a hydrate, preferably hydrazine monohydrate, with a compounds of formula XXV, wherein X, A, R₁ and R₃ are as defined in formula I above, and in which LG₂ and LG₆ are, independently from each other, leaving groups like, for example, chlorine, bromine or iodine (preferably chlorine or bromine), or an aryl- or (halo)alkylsulfonate such as trifluoromethanesulfonate. This transformation is preferably performed in an alcoholic solvent, such as methanol or ethanol, at temperatures between 20-150° C., preferably at temperatures ranging from room temperature to the boiling point of the reaction mixture, optionally under microwave irradiation.

Oxidation of compounds of formula XXV, wherein X, A, R₁ and R₃ are as defined in formula I above, and in which X is S (sulfide), and wherein LG₂ and LG₆ are, independently from each other, leaving groups like, for example, chlorine, bromine or iodine (preferably chlorine or bromine), or an aryl- or (halo)alkylsulfonate such as trifluoromethanesulfonate, with a suitable oxidizing agent (scheme 13), into compounds of formula XXV, wherein X is SO or SO₂ may be achieved under conditions already described above.

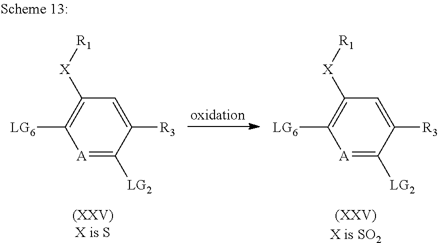

Compounds of formula XXV, wherein X, A, R₁ and R₃are as defined in formula I above, and in which X is S (sulfide), and wherein LG₂ and LG₆ are, independently from each other, leaving groups like, for example, chlorine, bromine or iodine (preferably chlorine or bromine), or an aryl- or (halo)alkylsulfonate such as trifluoromethanesulfonate,

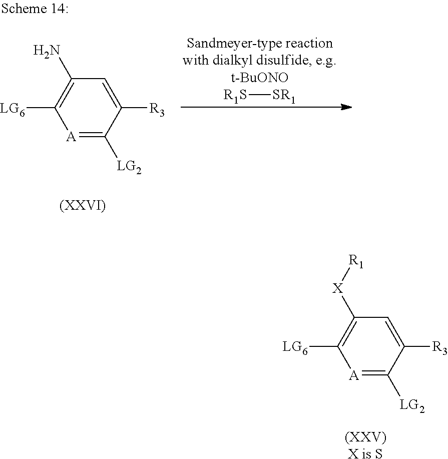

can be prepared (scheme 14) by reacting compounds of formula XXVI, wherein A and R₃are as defined in formula I above, and wherein LG₂ and LG₆ are, independently from each other, leaving groups like, for example, chlorine, bromine or iodine (preferably chlorine or bromine), or an aryl- or (halo)alkylsulfonate such as trifluoromethanesulfonate, with a nitrite, such as tert-butyl nitrite t-BuONO, isoamyl nitrite, or sodium nitrite in presence of a hydrohalic acid, and a disulfide R₁S—SR₁ or alternatively a thiol IRISH, wherein R₁ is as defined in formula I above, under Sandmeyer-type reaction conditions. This transformation is preferably performed in an inert solvent, such as acetonitrile or a halogenated solvent like 1,2-dichloroethane, at temperatures between 0-150° C., preferably at temperatures ranging from room temperature to the boiling point of the reaction mixture, optionally in the presence of copper salts.

Compounds of the formula XXVI, wherein A and R₃ are as defined in formula I above, and wherein LG₂ and LG₆ are, independently from each other, leaving groups like, for example, chlorine, bromine or iodine (preferably chlorine or bromine), or an aryl- or (halo)alkylsulfonate such as trifluoromethanesulfonate are known, commercially available or can be prepared by those skilled in the art.

Compounds of formula XXIII, or a salt thereof (such as a sodium, potassium or cesium carboxylate salt), wherein R₂ and G₂are as defined in formula I above, and in which LG₅ is a halogen leaving group like, for example, chlorine, bromine or iodine (preferably chlorine or iodine),

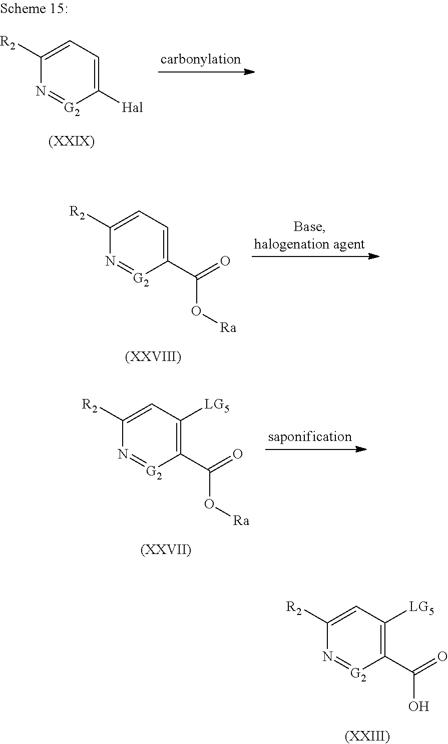

can be prepared (scheme 15) by saponification of the corresponding ester compounds of formula XXVII, wherein R₂ and G₂are as defined in formula I above, and in which LG₅ is a halogen leaving group like, for example, chlorine, bromine or iodine (preferably chlorine or iodine), and in which Ra is C₁-C₆alkyl, under conditions known to a person skilled in the art (using for example conditions such as: aqueous sodium, potassium or lithium hydroxide in methanol, ethanol or dioxane at room temperature, or up to refluxing conditions).

Compounds of formula XXVII, wherein R₂ and G₂are as defined in formula I above, and in which LG₅ is a halogen leaving group like, for example, chlorine, bromine or iodine (preferably chlorine or iodine), and in which Ra is C₁-C₆alkyl, can be prepared from compounds of formula XXIX, wherein R₂ and G₂ are as defined in formula I above, and in which Hal is a halogen such as chlorine, bromine or iodine (preferably chlorine or iodine) by methods found in, for example, WO 2016/020286 involving (scheme 15): 1) a carbonylation reaction, in which compounds of formula (XXIX) are reacted with carbon monoxide CO, in presence of metal catalyst such as a palladium catalyst (for example: palladium(II) acetate), in an alcohol RaOH solvent (typically methanol or ethanol), wherein Ra is C₁-C₆alkyl, and optionally in presence of a phosphine ligand, and optionally in presence of a base; followed by 2) a deprotonation/halogenation reaction, in which compounds of formula (XXVIII) are treated with a base, possibly at low temperature, and the generated organometallic species quenched with a halogen donor reagent (source of LG₅, wherein LG₅ is halogen leaving group like, for example, chlorine, bromine or iodine).

Compounds of the formula XXIX, wherein R₂ and G₂ are as defined in formula I above, and in which Hal is a halogen such as chlorine, bromine or iodine, are known, commercially available or can be prepared by those skilled in the art.

Compounds of formula I, wherein R₆ is labeled as R₆₍ₐₘᵢₙₒ₎, define the particular subgroup of compounds of formula I, wherein R₆ is amino (NH₂), hydroxyl-amino (NHOH) or NR₇R₈, wherein R₇ is C₁-C₄alkyl, preferably methyl, and R₈ is hydrogen or C₁-C₄alkyl, preferably hydrogen or methyl. Compounds of formula (Ie), wherein R₆ is R₆₍ₐₘᵢₙₒ₎, and wherein X, A, R₁, R₂, R₃ and G₂are as defined in formula I above, and in which R₄ is halogen, preferably chlorine or bromine,

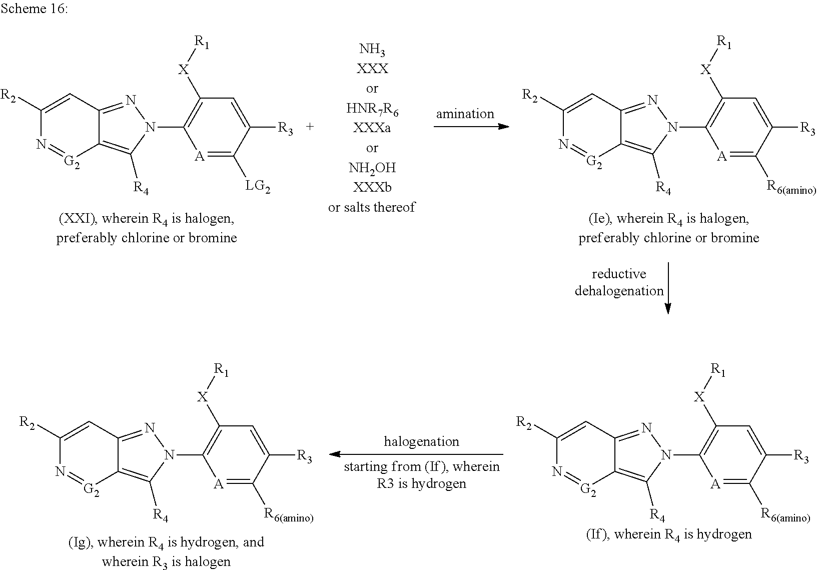

can be prepared (scheme 16) by an amination reaction, which involves for example, reacting compounds of formula XXI, wherein X, A, R₁, R₂, R₃ and G₂are as defined in formula I above, and wherein R₄ is halogen, preferably chlorine or bromine, and in which LG₂ is a leaving group like, for example, chlorine, bromine or iodine (preferably chlorine or bromine), or an aryl- or (halo)alkylsulfonate such as trifluoromethanesulfonate, with amino reagents of formula XXX (ammonia), formula XXXb (hydroxyl-amine), or formula XXXa (e.g. methylamine as a representative of C₁-C₄alkylamines H₂NR₇ [R₈ is hydrogen)], or dimethylamine as a representative of C₁-C₄dialkylamines HNR₇R₈), or corresponding salts thereof (such as a hydrohalide salt, preferably a hydrochloride or a hydrobromide salt, or any other equivalent salt). The source for the reagent XXX may be ammonia NH₃ or an ammonia equivalent such as for example ammonium hydroxide NH₄OH, ammonium chloride NH₄Cl, ammonium acetate NH₄OAc, ammonium carbonate (NH₄)₂CO₃, and other NH₃ surrogates. This transformation is preferably performed in suitable solvents (or diluents) such as alcohols, amides, esters, ethers, nitriles and water, particularly preferred are methanol, ethanol, 2,2,2-trifluoroethanol, propanol, iso-propanol, N,N-dimethylformamide, N,N-dimethylacetamide, dioxane, tetrahydrofuran, dimethoxyethane, acetonitrile, ethyl acetate, water or mixtures thereof, optionally in presence of a base, at temperatures between 0-150° C., preferably at temperatures ranging from room temperature to the boiling point of the reaction mixture, optionally under microwave irradiation.

Compounds of formula (If), wherein R₆ is R₆₍ₐₘᵢₙₒ₎ as defined above, and wherein X, A, R₁, R₂, R₃ and G₂ are as defined in formula I above, and in which R₄ is hydrogen, can be prepared (scheme 16) from compounds of formula (Ie), wherein R₆ is R₆₍ₐₘᵢₙₒ₎ as defined above, and wherein X, A, R₁, R₂, R₃ and G₂are as defined in formula I above, and in which R₄ is halogen, preferably chlorine or bromine, by means of a reductive dehalogenation under conditions already described above (see scheme 7).

Compounds of formula (Ig), wherein R₆ is R₆₍ₐₘᵢₙₒ₎ as defined above, and wherein R₃ is halogen (preferably chlorine, bromine or iodine), and wherein X, A, R₁, R₂ and G₂are as defined in formula I above, and in which R₄ is hydrogen, can be prepared (scheme 16) by a halogenation reaction, which involves for example, reacting compounds of formula (If), wherein R₆ is R₆₍ₐₘᵢₙₒ₎ as defined above, and wherein R₃ is hydrogen, and wherein X, A, R₁, R₂ and G₂are as defined in formula I above, and in which R₄ is hydrogen, with halogenating reagents such as N-chlorosuccinimide (NCS), N-bromosuccinimide (NBS) or N-iodosuccinimide (NIS), or alternatively chlorine, bromine or iodine. Such halogenation reactions are carried out in an inert solvent, such as chloroform, carbon tetrachloride, 1,2-dichloroethane, acetic acid, ethers, acetonitrile or N,N-dimethylformamide, at temperatures between 20-200° C., preferably room temperature to 100° C.

Compounds of formula I, wherein R₆ is labeled as R_{6(alkoxy)}, define the particular subgroup of compounds of formula I, wherein R₆ is C₁-C₆alkoxy, preferably methoxy or ethoxy.

Compounds of formula (Ih), wherein X, A, R₁, R₂, R₃ and G₂are as defined in formula I above, and in which R₄ is halogen, preferably chlorine or bromine,

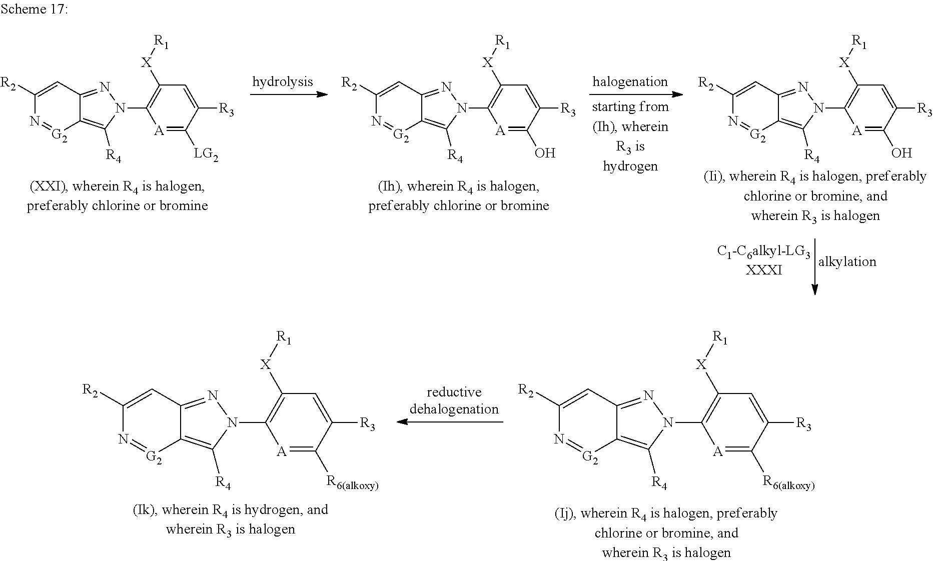

can be prepared (scheme 17) by a hydrolysis reaction, which involves for example, reacting compounds of formula XXI, wherein X, A, R₁, R₂, R₃ and G₂are as defined in formula I above, and wherein R₄ is halogen, preferably chlorine or bromine, and in which LG₂ is a leaving group like, for example, chlorine, bromine or iodine (preferably chlorine or bromine), or an aryl- or (halo)alkylsulfonate such as trifluoromethanesulfonate, with potassium, sodium or lithium hydroxide in solvents such as water, or water in mixtures with, for example, alcohols (eg. methanol, ethanol, isopropanol, or higher boiling linear or branched alcohols) or ethers (such as dioxane, tetrahydrofuran or dimethoxyethane), at temperatures between 20-180° C., preferably at temperatures ranging from room temperature to the boiling point of the reaction mixture, optionally under microwave irradiation.

Compounds of formula (Ii), wherein R₃ is halogen (preferably chlorine, bromine or iodine), and wherein X, A, R₁, R₂ and G₂are as defined in formula I above, and in which R₄ is halogen, preferably chlorine or bromine, can be prepared (scheme 17) by a halogenation reaction, which involves for example, reacting compounds of formula (Ih), wherein R₃ is hydrogen, and wherein X, A, R₁, R₂ and G₂are as defined in formula I above, and in which R₄ is halogen, preferably chlorine or bromine, with halogenating reagents such as N-chlorosuccinimide (NCS), N-bromosuccinimide (NBS) or N-iodosuccinimide (NIS), or alternatively chlorine, bromine or iodine, under conditions already described above (see scheme 16).

Compounds of formula (IA wherein R₆ is R_{6(alkoxy)}, and wherein R₃ is halogen (preferably chlorine, bromine or iodine), and wherein X, A, R₁, R₂ and G₂are as defined in formula I above, and in which R₄ is halogen, preferably chlorine or bromine, can be prepared (scheme 17) by an alkylation reaction, which involves for example, treating compounds of formula (Ii), wherein R₃ is halogen (preferably chlorine, bromine or iodine), and wherein X, A, R₁, R₂ and G₂are as defined in formula I above, and in which R₄ is halogen, preferably chlorine or bromine, with an alkylating agent of the formula [C₁-C₆alkyl-LG₃] XXXI, wherein LG₃ is a leaving group such as chlorine, bromine or iodine (preferably bromine or iodine), or an aryl- or (halo)alkylsulfonate such as trifluoromethanesulfonate. Typical examples of such alkylating agents are methyl iodide or ethyl bromide, alternatively other known reagents equivalent to XXXI may be employed, such as dimethyl sulfate. This transformation is preferably performed in presence of a suitable base, such as alkali metal carbonates, for example sodium carbonate, cesium carbonate and potassium carbonate, or alkali metal hydrides such as sodium hydride, or alkali metal hydroxides such as sodium hydroxide and potassium hydroxide, in an inert solvent at temperatures preferably between −10 and 180° C., preferably between 0-120° C., optionally under microwave irradiation. Examples of solvent to be used include ethers such as THF, ethylene glycol dimethyl ether, tert-butylmethyl ether, and 1,4-dioxane, aromatic hydrocarbons such as toluene and xylene, nitriles such as acetonitrile or polar aprotic solvents such as N,N-dimethylformamide, N,N-dimethylacetamide, N-methyl-2-pyrrolidone or dimethyl sulfoxide.

Compounds of formula (Ik), wherein R₆ is R_{6(alkoxy)}, and wherein R₃ is halogen (preferably chlorine, bromine or iodine), and wherein X, A, R₁, R₂ and G₂are as defined in formula I above, and in which R₄ is hydrogen can be prepared (scheme 17) from compounds of formula (IA wherein R₆ is R_{6(alkoxy)}, and wherein R₃ is halogen (preferably chlorine, bromine or iodine), and wherein X, A, R₁, R₂ and G₂are as defined in formula I above, and in which R₄ is halogen, preferably chlorine or bromine, by means of a reductive dehalogenation under conditions already described above (see scheme 7).

The oxidation state of sulfur-containing functionalities in substituents R₂ and R₆ of compounds of the formula I, and of any intermediates listed above required for the preparation of such compounds of the formula I, may be easily adapted from the sulfide oxidation state into the sulfoxide or sulfone level by means of an oxidation reaction involving reagents such as, for example, m-chloroperbenzoic acid (MCPBA), oxone, sodium periodate, sodium hypochlorite or tert-butyl hypochlorite amongst many others, under conditions already described above. As an example, such an oxidation protocol can be applied for the preparation of sulfone P28 from sulfide P27 (listed below in Table P).

The reactants can be reacted in the presence of a base. Examples of suitable bases are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylammonium hydroxide and 1,8-diazabicyclo[5.4.0]undec-7-ene (DBU).

The reactants can be reacted with each other as such, i.e. without adding a solvent or diluent. In most cases, however, it is advantageous to add an inert solvent or diluent or a mixture of these. If the reaction is carried out in the presence of a base, bases which are employed in excess, such as triethylamine, pyridine, N-methylmorpholine or N,N-diethylaniline, may also act as solvents or diluents.

The reaction is advantageously carried out in a temperature range from approximately −80° C. to approximately +140° C., preferably from approximately −30° C. to approximately +100° C., in many cases in the range between ambient temperature and approximately +80° C.

A compound of formula I can be converted in a manner known per se into another compound of formula I by replacing one or more substituents of the starting compound of formula I in the customary manner by (an)other substituent(s) according to the invention.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of compounds of formula I can be prepared in a manner known per se. Thus, for example, acid addition salts of compounds of formula I are obtained by treatment with a suitable acid or a suitable ion exchanger reagent and salts with bases are obtained by treatment with a suitable base or with a suitable ion exchanger reagent.

Salts of compounds of formula I can be converted in the customary manner into the free compounds I, acid addition salts, for example, by treatment with a suitable basic compound or with a suitable ion exchanger reagent and salts with bases, for example, by treatment with a suitable acid or with a suitable ion exchanger reagent.

Salts of compounds of formula I can be converted in a manner known per se into other salts of compounds of formula I, acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formula I, which have salt-forming properties can be obtained in free form or in the form of salts.

The compounds of formula I and, where appropriate, the tautomers thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and hereinbelow, even when stereochemical details are not mentioned specifically in each case.

Diastereomer mixtures or racemate mixtures of compounds of formula I, in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

N-oxides can be prepared by reacting a compound of the formula I with a suitable oxidizing agent, for example the H₂O₂/urea adduct in the presence of an acid anhydride, e.g. trifluoroacetic anhydride. Such oxidations are known from the literature, for example from J. Med. Chem., 32 (12), 2561-73, 1989 or WO 00/15615.

It is advantageous to isolate or synthesize in each case the biologically more effective isomer, for example enantiomer or diastereomer, or isomer mixture, for example enantiomer mixture or diastereomer mixture, if the individual components have a different biological activity. The compounds of formula I and, where appropriate, the tautomers thereof, in each case in free form or in salt form, can, if appropriate, also be obtained in the form of hydrates and/or include other solvents, for example those which may have been used for the crystallization of compounds which are present in solid form.

The compounds according to the following Tables 1 to 2 below can be prepared according to the methods described above. The examples which follow are intended to illustrate the invention and show preferred compounds of formula I. “Ph” represents the phenyl group.

Table 1: This table discloses the 43 compounds of the formula I-1a:

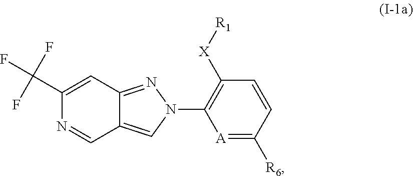

TABLE 1

| Comp. No | X | R₁ | R₆ | A |
|---|---|---|---|---|
| 1.001 | S | —CH₂CH₃ | 4-Cl—Ph— | CH |
| 1.002 | S(O)₂ | —CH₂CH₃ | 4-Cl—Ph— | CH |
| 1.003 | S | —CH₂CH₃ | 4-Cl—Ph— | N |
| 1.004 | S(O)₂ | —CH₂CH₃ | 4-Cl—Ph— | N |
| 1.005 | S | —CH₂CH₃ | 3-F—Ph— | CH |
| 1.006 | S(O)₂ | —CH₂CH₃ | 3-F—Ph— | CH |
| 1.007 | S | —CH₂CH₃ | 3-F—Ph— | N |
| 1.008 | S(O)₂ | —CH₂CH₃ | 3F—Ph— | N |
| 1.009 | S | —CH₂CH₃ | 3,5-diF—Ph— | CH |
| 1.010 | S(O)₂ | —CH₂CH₃ | 3,5-diF—Ph— | CH |
| 1.011 | S | —CH₂CH₃ | 3,5-diF—Ph— | N |
| 1.012 | S(O)₂ | —CH₂CH₃ | 3,5-diF—Ph— | N |
| 1.013 | S | —CH₂CH₃ | pyrazol-1-yl | CH |
| 1.014 | S(O)₂ | —CH₂CH₃ | pyrazol-1-yl | CH |
| 1.015 | S | —CH₂CH₃ | pyrazol-1-yl | N |
| 1.016 | S(O)₂ | —CH₂CH₃ | pyrazol-1-yl | N |
| 1.017 | S | —CH₂CH₃ | 4-cyano-pyrazol-1-yl | CH |
| 1.018 | S(O)₂ | —CH₂CH₃ | 4-cyano-pyrazol-1-yl | CH |
| 1.019 | S | —CH₂CH₃ | 4-cyano-pyrazol-1-yl | N |
| 1.020 | S(O)₂ | —CH₂CH₃ | 4-cyano-pyrazol-1-yl | N |
| 1.021 | S | —CH₂CH₃ | 3-cyano-pyrazol-1-yl | CH |
| 1.022 | S(O)₂ | —CH₂CH₃ | 3-cyano-pyrazol-1-yl | CH |
| 1.023 | S | —CH₂CH₃ | 3-cyano-pyrazol-1-yl | N |
| 1.024 | S(O)₂ | —CH₂CH₃ | 3-cyano-pyrazol-1-yl | N |
| 1.025 | S | —CH₂CH₃ | 4-chloro-pyrazol-1-yl | CH |
| 1.026 | S(O)₂ | —CH₂CH₃ | 4-chloro-pyrazol-1-yl | CH |
| 1.027 | S | —CH₂CH₃ | 4-chloro-pyrazol-1-yl | N |
| 1.028 | S(O)₂ | —CH₂CH₃ | 4-chloro-pyrazol-1-yl | N |
| 1.029 | S | —CH₂CH₃ | 3-chloro-pyrazol-1-yl | CH |
| 1.030 | S(O)₂ | —CH₂CH₃ | 3-chloro-pyrazol-1-yl | CH |
| 1.031 | S | —CH₂CH₃ | 3-chloro-pyrazol-1-yl | N |
| 1.031 | S(O)₂ | —CH₂CH₃ | 3-chloro-pyrazol-1-yl | N |
| 1.032 | S | —CH₂CH₃ | 1,2,4-triazol-1-yl | CH |
| 1.033 | S(O)₂ | —CH₂CH₃ | 1,2,4-triazol-1-yl | CH |
| 1.034 | S | —CH₂CH₃ | 1,2,4-triazol-1-yl | N |
| 1.035 | S(O)₂ | —CH₂CH₃ | 1,2,4-triazol-1-yl | N |
| 1.036 | S | —CH₂CH₃ | 3-chloro-1,2,4-triazol-1-yl | CH |
| 1.037 | S(O)₂ | —CH₂CH₃ | 3-chloro-1,2,4-triazol-1-yl | CH |
| 1.038 | S | —CH₂CH₃ | 3-chloro-1,2,4-triazol-1-yl | N |
| 1.039 | S(O)₂ | —CH₂CH₃ | 3-chloro-1,2,4-triazol-1-yl | N |
| 1.040 | S | —CH₂CH₃ | 3-cyano-1,2,4-triazol-1-yl | CH |
| 1.041 | S(O)₂ | —CH₂CH₃ | 3-cyano-1,2,4-triazol-1-yl | CH |
| 1.042 | S | —CH₂CH₃ | 3-cyano-1,2,4-triazol-1-yl | N |
| 1.043 | S(O)₂ | —CH₂CH₃ | 3-cyano-1,2,4-triazol-1-yl | N |

and the N-oxides of the compounds of Table 1.

Table 2: This table discloses 43 compounds of formula I-2b:

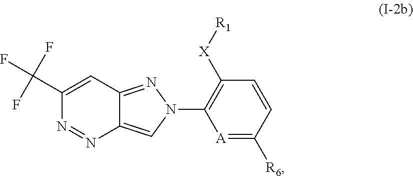

TABLE 2

| Comp. No | X | R₁ | R₆ | A |
|---|---|---|---|---|
| 2.001 | S | —CH₂CH₃ | 4-Cl—Ph— | CH |
| 2.002 | S(O)₂ | —CH₂CH₃ | 4-Cl—Ph— | CH |
| 2.003 | S | —CH₂CH₃ | 4-Cl—Ph— | N |
| 2.004 | S(O)₂ | —CH₂CH₃ | 4-Cl—Ph— | N |
| 2.005 | S | —CH₂CH₃ | 3-F—Ph— | CH |
| 2.006 | S(O)₂ | —CH₂CH₃ | 3-F—Ph— | CH |
| 2.007 | S | —CH₂CH₃ | 3-F—Ph— | N |
| 2.008 | S(O)₂ | —CH₂CH₃ | 3F—Ph— | N |
| 2.009 | S | —CH₂CH₃ | 3,5-diF—Ph— | CH |
| 2.010 | S(O)₂ | —CH₂CH₃ | 3,5-diF—Ph— | CH |
| 2.011 | S | —CH₂CH₃ | 3,5-diF—Ph— | N |
| 2.012 | S(O)₂ | —CH₂CH₃ | 3,5-diF—Ph— | N |
| 2.013 | S | —CH₂CH₃ | pyrazol-1-yl | CH |
| 2.014 | S(O)₂ | —CH₂CH₃ | pyrazol-1-yl | CH |
| 2.015 | S | —CH₂CH₃ | pyrazol-1-yl | N |
| 2.016 | S(O)₂ | —CH₂CH₃ | pyrazol-1-yl | N |
| 2.017 | S | —CH₂CH₃ | 4-cyano-pyrazol-1-yl | CH |
| 2.018 | S(O)₂ | —CH₂CH₃ | 4-cyano-pyrazol-1-yl | CH |
| 2.019 | S | —CH₂CH₃ | 4-cyano-pyrazol-1-yl | N |
| 2.020 | S(O)₂ | —CH₂CH₃ | 4-cyano-pyrazol-1-yl | N |
| 2.021 | S | —CH₂CH₃ | 3-cyano-pyrazol-1-yl | CH |
| 2.022 | S(O)₂ | —CH₂CH₃ | 3-cyano-pyrazol-1-yl | CH |
| 2.023 | S | —CH₂CH₃ | 3-cyano-pyrazol-1-yl | N |
| 2.024 | S(O)₂ | —CH₂CH₃ | 3-cyano-pyrazol-1-yl | N |
| 2.025 | S | —CH₂CH₃ | 4-chloro-pyrazol-1-yl | CH |
| 2.026 | S(O)₂ | —CH₂CH₃ | 4-chloro-pyrazol-1-yl | CH |
| 2.027 | S | —CH₂CH₃ | 4-chloro-pyrazol-1-yl | N |
| 2.028 | S(O)₂ | —CH₂CH₃ | 4-chloro-pyrazol-1-yl | N |
| 2.029 | S | —CH₂CH₃ | 3-chloro-pyrazol-1-yl | CH |
| 2.030 | S(O)₂ | —CH₂CH₃ | 3-chloro-pyrazol-1-yl | CH |
| 2.031 | S | —CH₂CH₃ | 3-chloro-pyrazol-1-yl | N |
| 2.031 | S(O)₂ | —CH₂CH₃ | 3-chloro-pyrazol-1-yl | N |
| 2.032 | S | —CH₂CH₃ | 1,2,4-triazol-1-yl | CH |
| 2.033 | S(O)₂ | —CH₂CH₃ | 1,2,4-triazol-1-yl | CH |
| 2.034 | S | —CH₂CH₃ | 1,2,4-triazol-1-yl | N |
| 2.035 | S(O)₂ | —CH₂CH₃ | 1,2,4-triazol-1-yl | N |
| 2.036 | S | —CH₂CH₃ | 3-chloro-1,2,4-triazol-1-yl | CH |
| 2.037 | S(O)₂ | —CH₂CH₃ | 3-chloro-1,2,4-triazol-1-yl | CH |
| 2.038 | S | —CH₂CH₃ | 3-chloro-1,2,4-triazol-1-yl | N |
| 2.039 | S(O)₂ | —CH₂CH₃ | 3-chloro-1,2,4-triazol-1-yl | N |
| 2.040 | S | —CH₂CH₃ | 3-cyano-1,2,4-triazol-1-yl | CH |
| 2.041 | S(O)₂ | —CH₂CH₃ | 3-cyano-1,2,4-triazol-1-yl | CH |
| 2.042 | S | —CH₂CH₃ | 3-cyano-1,2,4-triazol-1-yl | N |
| 2.043 | S(O)₂ | —CH₂CH₃ | 3-cyano-1,2,4-triazol-1-yl | N |

and the N-oxides of the compounds of Table 2.

The compounds of formula I according to the invention are preventively and/or curatively valuable active ingredients in the field of pest control, even at low rates of application, which have a very favorable biocidal spectrum and are well tolerated by warm-blooded species, fish and plants. The active ingredients according to the invention act against all or individual developmental stages of normally sensitive, but also resistant, animal pests, such as insects or representatives of the order Acarina. The insecticidal or acaricidal activity of the active ingredients according to the invention can manifest itself directly, i. e. in destruction of the pests, which takes place either immediately or only after some time has elapsed, for example during ecdysis, or indirectly, for example in a reduced oviposition and/or hatching rate.

Examples of the abovementioned animal pests are:

from the order Acarina, for example,

Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;

from the order Anoplura, for example,

Haematopinus spp., Linognathus spp., Pediculus spp., Pemphigus spp. and Phylloxera spp.; from the order Coleoptera, for example,

Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemLineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.; from the order Diptera, for example,

Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;

from the order Hemiptera, for example,

Acanthocoris scabrator, Acrosternum spp, Adelphocoris lineolatus, Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euchistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens; Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maid is, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris;

from the order Hymenoptera, for example,

Acromyrmex, Arge spp, Atta spp., Cephus spp., Diprion spp., Diprionidae, Gilpinia polytoma, Hoplo-campa spp., Lasius spp., Monomorium pharaonis, Neodiprion spp., Pogonomyrmex spp, Slenopsis invicta, Solenopsis spp. and Vespa spp.;

from the order Isoptera, for example,

Coptotermes spp, Corniternes cumulans, Incisitermes spp, Macrotermes spp, Mastotermes spp, Microtermes spp, Reticulitermes spp.; Solenopsis geminate

from the order Lepidoptera, for example,

Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Eldana saccharina, Ephestia spp., Epinotia spp, Estigmene acrea, Etiella zinckinella, Eucosma spp., Eupoecilia ambiguella, Euproctis spp., Euxoa spp., Feltiajaculiferia, Gra-pholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypi-ela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;

from the order Mallophaga, for example,

Damalinea spp. and Trichodectes spp.;

from the order Orthoptera, for example,

Blatta spp., Blattella spp., Gryllotalpa spp., Leucophaea maderae, Locusta spp., Neocurtilla hexadactyla, Periplaneta spp., Scapteriscus spp, and Schistocerca spp.;

from the order Psocoptera, for example,

Liposcelis spp.;

from the order Siphonaptera, for example,

Ceratophyllus spp., Ctenocephalides spp. and Xenopsylla cheopis;

from the order Thysanoptera, for example,

Calliothrips Phaseoli, Frankliniella Spp., Heliothrips Spp, Hercinothrips Spp., Parthenothrips Spp, Scirtothrips aurantii, Sericothrips variabilis, Taeniothrips spp., Thrips spp;

from the order Thysanura, for example, Lepisma saccharina.

The active ingredients according to the invention can be used for controlling, i. e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.

The compositions and/or methods of the present invention may be also used on any ornamental and/or vegetable crops, including flowers, shrubs, broad-leaved trees and evergreens.

For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperflorens, B. tubereux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime), Coreopsis spp., Crassula coccinea, Cuphea ignea, Dahlia spp., Delphinium spp., Dicentra spectabilis, Dorotheantus spp.,

Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. Walleriana), (resines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Bellis spp., Pelargonium spp. (P. peltatum, P. Zonale), Viola spp. (pansy), Petunia spp., Phlox spp., Plecthranthus spp., Poinsettia spp., Parthenocissus spp. (P. quinquefolia, P. tricuspidata), Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.

For example the invention may be used on any of the following vegetable species: Allium spp. (A. sativum, A. cepa, A. oschaninii, A. Porrum, A. ascalonicum, A. fistulosum), Anthriscus cerefolium, Apium graveolus, Asparagus officinalis, Beta vulgarus, Brassica spp. (B. Oleracea, B. Pekinensis, B. rapa), Capsicum annuum, Cicer arietinum, Cichorium endivia, Cichorum spp. (C. intybus, C. endivia), Citrillus lanatus, Cucumis spp. (C. sativus, C. melo), Cucurbita spp. (C. pepo, C. maxima), Cyanara spp. (C. scolymus, C. cardunculus), Daucus carota, Foeniculum vulgare, Hypericum spp., Lactuca sativa, Lycopersicon spp. (L. esculentum, L. lycopersicum), Mentha spp., Ocimum basilicum, Petroselinum crispum, Phaseolus spp. (P. vulgaris, P. coccineus), Pisum sativum, Raphanus sativus, Rheum rhaponticum, Rosemarinus spp., Salvia spp., Scorzonera hispanica, Solanum melongena, Spinacea oleracea, Valerianella spp. (V. locusta, V. eriocarpa) and Vicia faba.

Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatoes) and Chilo supressalis (preferably in rice).

In a further aspect, the invention may also relate to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

The compounds of the invention may also have activity against the molluscs. Examples of which include, for example, Ampullariidae; Arion (A. ater, A. circumscriptus, A. hortensis, A. rufus); Bradybaenidae (Bradybaena fruticum); Cepaea (C. hortensis, C. Nemoralis); ochlodina; Deroceras (D. agrestis, D. empiricorum, D. laeve, D. reticulatum); Discus (D. rotundatus); Euomphalia; Galba (G. trunculata); Helicelia (H. itala, H. obvia); Helicidae Helicigona arbustorum); Helicodiscus; Helix (H. aperta); Limax (L. cinereoniger, L. flavus, L. marginatus, L. maximus, L. tenellus); Lymnaea; Milax (M. gagates, M. marginatus, M. sowerbyi); Opeas; Pomacea (P. canaticulata); Vallonia and Zanitoides.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popilliae; or insecticidal proteins from Bacillus thuringiensis, such as δ-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by δ-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810). Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. Cry1-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and moths (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.

2. Bt176 Maize from Syngenta Seeds SAS, Chemin de l′Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.

3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.

4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.

5. IPC 531 Cotton from Monsanto Europe S.A. 270-272 Avenue de Tervuren, B-1150 Brussels, Belgium, registration number C/ES/96/02.

6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.

7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit and Nachhaltigkeit, Zentrum BATS, Clarastrasse 13, 4058 Basel, Switzerland) Report 2003, (http://bats.ch).

The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF—YB or other proteins known in the art.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Further areas of use of the compositions according to the invention are the protection of stored goods and store rooms and the protection of raw materials, such as wood, textiles, floor coverings or buildings, and also in the hygiene sector, especially the protection of humans, domestic animals and productive livestock against pests of the mentioned type.

The present invention also provides a method for controlling pests (such as mosquitoes and other disease vectors; see also http://www.who.int/malaria/vector_control/irs/en/). In one embodiment, the method for controlling pests comprises applying the compositions of the invention to the target pests, to their locus or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which can be used in the manufacture of) netting, clothing, bedding, curtains and tents.

In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on a substrate such as a fabric material in the form of (or which can be used in the manufacture of) netting, clothing, bedding, curtains and tents.

Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO 2008/151984, WO 2003/034823, U.S. Pat. No. 5,631,072, WO 2005/64072, WO 2006/128870, EP 1724392, WO 2005/113886 or WO 2007/090739.

Further areas of use of the compositions according to the invention are the field of tree injection/trunk treatment for all ornamental trees as well all sort of fruit and nut trees.

In the field of tree injection/trunk treatment, the compounds according to the present invention are especially suitable against wood-boring insects from the order Lepidoptera as mentioned above and from the order Coleoptera, especially against woodborers listed in the following tables A and B:

TABLE A
Examples of exotic woodborers of economic importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE B
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus vittaticolllis | Apple, Pear, Cranberry, |
| | | Serviceberry, Hawthorn |
| | Chrysobothris femorata | Apple, Apricot, Beech, Boxelder, |
| | | Cherry, Chestnut, Currant, Elm, |
| | | Hawthorn, Hackberry, Hickory, |
| | | Horsechestnut, Linden, Maple, |
| | | Mountain-ash, Oak, Pecan, Pear, |
| | | Peach, Persimmon, Plum, Poplar, |
| | | Quince, Redbud, Serviceberry, |
| | | Sycamore, Walnut, Willow |
| | Texania campestris | Basswood, Beech, Maple, Oak, |
| | | Sycamore, Willow, Yellow-poplar |
| Cerambycidae | Goes pulverulentus | Beech, Elm, Nuttall, Willow, Black |
| | | oak, Cherrybark oak, Water oak, |
| | | Sycamore |
| | Goes tigrinus | Oak |
| | Neoclytus acuminatus | Ash, Hickory, Oak, Walnut, Birch, |
| | | Beech, Maple, Eastern |
| | | hophornbeam, Dogwood, |
| | | Persimmon, Redbud, Holly, |
| | | Hackberry, Black locust, |
| | | Honeylocust, Yellow-poplar, |
| | | Chestnut, Osage-orange, Sassafras, |
| | | Lilac, Mountain-mahogany, Pear, |
| | | Cherry, Plum, Peach, Apple, Elm, |
| | | Basswood, Sweetgum |
| | Neoptychodes trilineatus | Fig, Alder, Mulberry, Willow, Netleaf |
| | | hackberry |
| | Oberea ocellata | Sumac, Apple, Peach, Plum, Pear, |
| | | Currant, Blackberry |
| | Oberea tripunctata | Dogwood, Viburnum, Elm, |
| | | Sourwood, Blueberry, |
| | | Rhododendron, Azalea, Laurel, |
| | | Poplar, Willow, Mulberry |
| | Oncideres cingulata | Hickory, Pecan, Persimmon, Elm, |
| | | Sourwood, Basswood, Honeylocust, |
| | | Dogwood, Eucalyptus, Oak, |
| | | Hackberry, Maple, Fruit trees |
| | Saperda calcarata | Poplar |
| | Strophiona nitens | Chestnut, Oak, Hickory, Walnut, |
| | | Beech, Maple |
| Scolytidae | Corthylus columbianus | Maple, Oak, Yellow-poplar, Beech, |
| | | Boxelder, Sycamore, Birch, |
| | | Basswood, Chestnut, Elm |
| | Dendroctonus frontalis | Pine |
| | Dryocoetes betulae | Birch, Sweetgum, Wild cherry, |
| | | Beech, Pear |
| | Monarthrum fasciatum | Oak, Maple, Birch, Chestnut, |
| | | Sweetgum, Blackgum, Poplar, |
| | | Hickory, Mimosa, Apple, Peach, Pine |
| | Phloeotribus liminaris | Peach, Cherry, Plum, Black cherry, |
| | | Elm, Mulberry, Mountain-ash |
| | Pseudopityophthorus pruinosus | Oak, American beech, Black cherry, |
| | | Chickasaw plum, Chestnut, Maple, |
| | | Hickory, Hornbeam, Hophornbeam |
| Sesiidae | Paranthrene simulans | Oak, American chestnut |
| | Sannina uroceriformis | Persimmon |
| | Synanthedon exitiosa | Peach, Plum, Nectarine, Cherry, |
| | | Apricot, Almond, Black cherry |
| | Synanthedon pictipes | Peach, Plum, Cherry, Beach, Black |
| | | Cherry |
| | Synanthedon rubrofascia | Tupelo |
| | Synanthedon scitula | Dogwood, Pecan, Hickory, Oak, |
| | | Chestnut, Beech, Birch, Black cherry, |
| | | Elm, Mountain-ash, Viburnum, |
| | | Willow, Apple, Loquat, Ninebark, |
| | | Bayberry |
| | Vitacea polistiformis | Grape |

The present invention may be also used to control any insect pests that may be present in turfgrass, including for example beetles, caterpillars, fire ants, ground pearls, millipedes, sow bugs, mites, mole crickets, scales, mealybugs ticks, spittlebugs, southern chinch bugs and white grubs. The present invention may be used to control insect pests at various stages of their life cycle, including eggs, larvae, nymphs and adults.

In particular, the present invention may be used to control insect pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass ataenius, A. spretulus), Maladera spp. (e.g. Asiatic garden beetle, M. castanea) and Tomarus spp.), ground pearls (Margarodes spp.), mole crickets (tawny, southern, and short-winged; Scapteriscus spp., Gryllotalpa africana) and leatherjackets (European crane fly, Tipula spp.).

The present invention may also be used to control insect pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control insect pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs.

The present invention may also be used to control other pests of turfgrass such as red imported fire ants (Solenopsis invicta) that create ant mounds in turf.

In the hygiene sector, the compositions according to the invention are active against ectoparasites such as hard ticks, soft ticks, mange mites, harvest mites, flies (biting and licking), parasitic fly larvae, lice, hair lice, bird lice and fleas.

Examples of such parasites are:

Of the order Anoplurida: Haematopinus spp., Linognathus spp., Pediculus spp. and Phtirus spp., Solenopotes spp.

Of the order Mallophagida: Trimenopon spp., Menopon spp., Trinoton spp., Bovicola spp., Werneckiella spp., Lepikentron spp., Damalina spp., Trichodectes spp. and Felicola spp.

Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

Of the order Siphonapterida, for example Pulex spp., Ctenocephalides spp., Xenopsylla spp., Ceratophyllus spp.

Of the order Heteropterida, for example Cimex spp., Triatoma spp., Rhodnius spp., Panstrongylus spp.

Of the order Blattarida, for example Blatta orientalis, Periplaneta americana, Blattelagermanica and Supella spp.

Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

Of the orders Actinedida (Prostigmata) and Acaridida (Astigmata), for example Acarapis spp., Cheyletiella spp., Ornithocheyletia spp., Myobia spp., Psorergatesspp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compositions according to the invention are also suitable for protecting against insect infestation in the case of materials such as wood, textiles, plastics, adhesives, glues, paints, paper and card, leather, floor coverings and buildings.

The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec.,Tryptodendron spec., Apate monachus, Bostrychus capucins, Heterobostrychus brunneus, Sinoxylon spec. and Dinoderus minutus, and also hymenopterans such as Sirex juvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The compounds according to the invention can be used as pesticidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First

Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood N.J. (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively).

Many oil derivatives are known from the Compendium of Herbicide Adjuvants, 10th Edition, Southern Illinois University, 2010.

The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance.

Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 I/ha, especially from 10 to 1000 I/ha.

Preferred formulations can have the following compositions (weight %):

Emulsifiable Concentrates:

active ingredient: 1 to 95%, preferably 60 to 90%

surface-active agent: 1 to 30%, preferably 5 to 20%

liquid carrier: 1 to 80%, preferably 1 to 35%

Dusts:

active ingredient: 0.1 to 10%, preferably 0.1 to 5%

Suspension Concentrates:

active ingredient: 5 to 75%, preferably 10 to 50%

water: 94 to 24%, preferably 88 to 30%

surface-active agent: 1 to 40%, preferably 2 to 30%

Wettable Powders:

active ingredient: 0.5 to 90%, preferably 1 to 80%

surface-active agent: 0.5 to 20%, preferably 1 to 15%

solid carrier: 5 to 95%, preferably 15 to 90%

Granules:

active ingredient: 0.1 to 30%, preferably 0.1 to 15%

solid carrier: 99.5 to 70%, preferably 97 to 85%

The following Examples further illustrate, but do not limit, the invention.

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The combination is thoroughly mixed with the adjuvants and the mixture is thoroughly ground in a suitable mill, affording powders that can be used directly for seed treatment.

| Emulsifiable concentrate | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether | 4% |
| (35 mol of ethylene oxide) | |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

| Extruder granules | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

| Coated granules | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

| Suspension concentrate | |
|---|---|
| active ingredients | 40% |
| propylene glycol | 10% |
| nonylphenol polyethylene glycol ether | 6% |
| (15 mol of ethylene oxide) | |
| Sodium lignosulfonate | 10% |
| carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Flowable concentrate for seed treatment

| | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one | 0.5% |
| (in the form of a 20% solution in water) | |
| monoazo-pigment calcium salt | 5% |
| Silicone oil (in the form of a 75% emulsion in water) | 0.2% |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Slow Release Capsule Suspension 28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

### PREPARATORY EXAMPLES

“Mp” means melting point in ° C. Free radicals represent methyl groups. ¹H NMR measurements were recorded on a Brucker 400 MHz spectrometer, chemical shifts are given in ppm relevant to a TMS standard. Spectra measured in deuterated solvents as indicated. Common abbreviations: aq=aqueous, min=minute, h=hour, sat=saturated, Rt=retention time, mCPBA=meta-chloroperoxybenzoic acid, MeOH=methanol, EtOH=ethanol, EtSH=ethanethiol, EA=ethyl acetate, NaHCO₃=sodium hydrogen carbonate, Na₂CO₃=sodium carbonate, HCl=hydrogen chloride, DCM or CH₂Cl₂=dichloromethane, Et₃N=triethylamine, DMF=N,N-dimethylformamide. Either one of the LCMS methods below was used to characterize the compounds. The characteristic LCMS values obtained for each compound were the retention time (“Rt”, recorded in minutes) and the measured molecular ion (M+H)⁺.

### LCMS Methods:

### Method 1:

Spectra were recorded on a Mass Spectrometer from Waters (ZQ Single quadrupole mass spectrometer) equipped with an electrospray source (Polarity: positive or negative ions, Capillary: 3.00 kV, Cone range: 30-60 V, Extractor: 2.00 V, Source Temperature: 150° C., Desolvation Temperature: 350° C., Cone Gas Flow: 0 L/Hr, Desolvation Gas Flow: 650 L/Hr, Mass range: 100 to 900 Da) and an Acquity UPLC from Waters: Binary pump, heated column compartment and diode-array detector. Solvent degasser, binary pump, heated column compartment and diode-array detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH: gradient: 0 min 0% B, 100% A; 1.2-1.5 min 100% B; Flow (ml/min) 0.85.

### Method 2:

Spectra were recorded on a Mass Spectrometer from Waters (SQD or ZQ Single quadrupole mass spectrometer) equipped with an electrospray source (Polarity: positive or negative ions, Capillary: 3.00 kV, Cone range: 30-60 V, Extractor: 2.00 V, Source Temperature: 150° C., Desolvation Temperature: 350° C., Cone Gas Flow: 0 L/Hr, Desolvation Gas Flow: 650 L/Hr, Mass range: 100 to 900 Da) and an Acquity UPLC from Waters: Binary pump, heated column compartment and diode-array detector. Solvent degasser, binary pump, heated column compartment and diode-array detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH; gradient: 0 min 0% B, 100% A; 2.7-3.0 min 100% B; Flow (ml/min) 0.85.

### Method 3:

Spectra were recorded on a Mass Spectrometer from Agilent Technologies (6410 Triple Quadruple Mass Spectrometer) equipped with an electrospray source (Polarity: Positive and Negative Polarity Switch, Capillary: 4.00 kV, Fragmentor: 100.00 V, Gas Temperature: 350° C., Gas Flow: 11 L/min, Nebulizer Gas: 45 psi, Mass range: 110-1000 Da, DAD Wavelength range: 210-400 nm). Column: KINETEX EVO C18, length 50 mm, diameter 4.6 mm, particle size 2.6 μm. Column oven temperature 40° C. Solvent gradient: A=Water with 0.1% formic acid: Acetonitrile (95:5 v/v). B=Acetonitrile with 0.1% formic acid. Gradient=0 min 90% A, 10% B; 0.9-1.8 min 0% A, 100% B, 2.2-2.5 min 90% A, 10% B. Flow rate 1.8 mL/min.

### Method 4:

Spectra were recorded on a Mass Spectrometer from Waters (Acquity SDS Mass Spectrometer) equipped with an electrospray source (Polarity: Positive and Negative Polarity Switch, Capillary: 3.00 kV, Cone Voltage: 41.00 V, Source temperature: 150° C., Desolvation Gas Flow: 1000 L/Hr, Desolvation temperature: 500° C., Gas Flow®Cone: 50 L/hr, Mass range: 110-800 Da, PDA wavelength range: 210-400 nm. Column: Acquity UPLC HSS T3 C₁₈, length 30 mm, diameter 2.1 mm, particle size 1.8 μm. Column oven temperature 40° C. Solvent gradient: A=Water with 0.1% formic acid: Acetonitrile (95:5 v/v). B=Acetonitrile with 0.05% formic acid. Gradient=0 min 90% A, 10% B; 0.2 min 50% A, 50% B; 0.7-1.3 min 0% A, 100% B; 1.4-1.6 min 90% A, 10% B. Flow rate 0.8 mL/min.

### Example P1: Preparation of 2-[6-(3,5-difluorophenyl)-3-ethylsulfonyl-2-pyridyl]-6-(trifluoromethyl)pyrazolo[4,3-c]pyridine (compound P1)

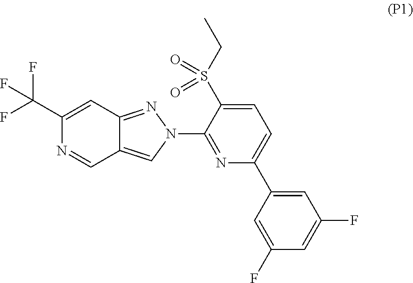 [SMILES: CCS(=O)(=O)c1ccc(-c2cc(F)cc(F)c2)nc1-n1cc2cnc(C(F)(F)F)cc2n1]

### Step P1.1: Preparation of 2-(3,5-difluorophenyl)-5-fluoro-pyridine

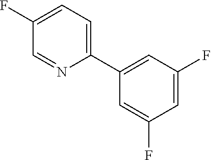 [SMILES: Fc1ccc(-c2cc(F)cc(F)c2)nc1]

A solution of 2-bromo-5-fluoro-pyridine (2.0 g, 11.4 mmol), (3,5-difluorophenyl)boronic acid (1.9 g, 12.0 mmol), aqueous 2M sodium carbonate (17 ml, 34 mmol) in dioxane (17 ml) was flushed with argon for 10 minutes. Tetrakis(triphenylphosphine) palladium (130 mg, 0.11 mmol) was added and the mixture was heated in the microwave at 90° C. for 45 minutes. After cooling, the reaction mixture was diluted with water and t-butyl methyl ether. The water phase was separated and extracted twice with t-butyl methyl ether. The combined organic phases were dried over sodium sulfate and concentrated in vacuo. The residue was purified over silica by flash column chromatography (cyclohexane) to afford 2-(3,5-difluorophenyl)-5-fluoro-pyridine as a solid, mp 63-65° C. LCMS (method 2): 210 (M+H)⁺, retention time 1.60 min. ¹H-NMR (CDCl₃, ppm) 6.84 (m, 1H), 7.49 (m, 3H), 7.69 (m, 1H), 8.54 (d, 1H).

### Step P1.2: Preparation of 2-(3,5-difluorophenyl)-5-ethylsulfonyl-1-oxido-pyridin-1-ium

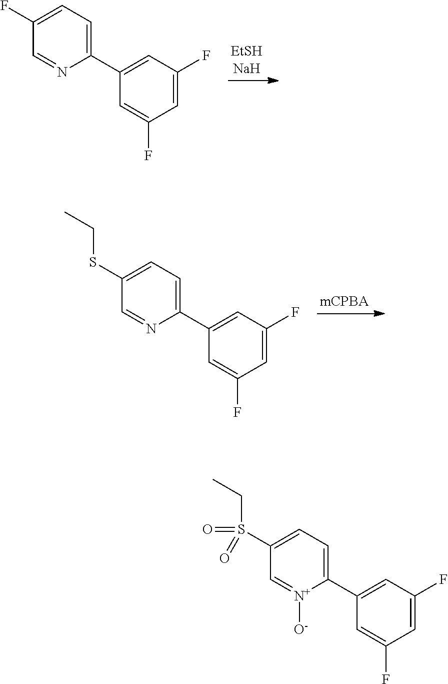 [SMILES: Fc1ccc(-c2cc(F)cc(F)c2)nc1 ; CCSc1ccc(-c2cc(F)cc(F)c2)nc1 ; CCS(=O)(=O)c1ccc(-c2cc(F)cc(F)c2)[n+]([O-])c1]

To a solution of 2-(3,5-difluorophenyl)-5-fluoro-pyridine (1.7 g, 8.13 mmol) in tetrahydrofuran (30 ml) at room temperature was added ethanethiol (1.36 ml, 17.9 mmol), followed by sodium hydride (715 mg, 60% in mineral oil, 17.9 mmol). The reaction mixture was heated to reflux for a total of 18 hours. After 6 hours, another ethanethiol portions (0.31 ml) was added to the mixture. After cooling, the reaction mixture was filtered over diatomaceous earth (Hyflo), the solid residue washed with ethyl acetate and the filtrate concentrated in vacuo to afford crude 2-(3,5-difluorophenyl)-5-ethylsulfanyl-pyridine. This material was used into the next step without further purification. LCMS (method 2): 252 (M+H)⁺, retention time 1.91 min. ¹H-NMR (CDCl₃, ppm) 1.36 (t, 3H), 3.01 (q, 2H), 6.84 (m, 1H), 7.51 (m, 2H), 7.60 (dd, 1H), 7.70 (d, 1H), 8.61 (dd, 1H).

To a solution of above crude 2-(3,5-difluorophenyl)-5-ethylsulfanyl-pyridine (2.18 g, 8.67 mmol) in dichloromethane (40 ml) was added mCPBA (75 wt % in water) (6.0 g, 26.07 mmol, 75%) in two portions and the mixture was stirred at room temperature for 60 hours, at 30° C. for one hour and at 40° C. for 4 hours. The reaction mixture was diluted with dichloromethane, washed successively with an aqueous sodium carbonate solution (4×), a 10% aqueous sodium bisulfite solution (3×), and brine, dried over sodium sulfate and concentrated under reduced pressure. The residue was purified over silica by flash column chromatography (30-85% ethyl acetate gradient in cyclohexane) to afford 2-(3,5-difluorophenyl)-5-ethylsulfonyl-1-oxido-pyridin-1-ium as a solid. LCMS (method 1): 300 (M+H)⁺, retention time 0.72 min. ¹H-NMR (CDCl₃, ppm) 1.40 (t, 3H), 3.23 (q, 2H), 6.99 (m, 1H), 7.44 (m, 2H), 7.62 (d, 1H), 7.71 (dd, 1H), 8.78 (d, 1H).

### Step P1.3: Preparation of 2-chloro-6-(3,5-difluorophenyl)-3-ethylsulfonyl-pyridine

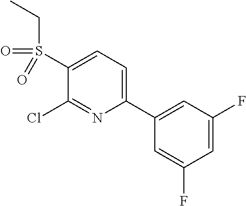 [SMILES: CCS(=O)(=O)c1ccc(-c2cc(F)cc(F)c2)nc1Cl]

2-(3,5-difluorophenyl)-5-ethylsulfonyl-1-oxido-pyridin-1-ium (0.5 g, 1.67 mmol) and phosphorus oxychloride (11 ml) were mixed and heated in the microwave at 130° C. for 33 minutes. The reaction mixture was concentrated in vacuo and the residue purified over silica by flash column chromatography (8-10% ethyl acetate gradient in cyclohexane) to afford 2-chloro-6-(3,5-difluorophenyl)-3-ethylsulfonyl-pyridine as a solid, mp 136-137° C. LCMS (method 1): 318/320 (M+H)⁺, retention time 1.03 min. ¹H-NMR (CDCl₃, ppm) δ 1.34 (t, 3H), 3.53 (q, 2H), 6.97 (m, 1H), 7.62 (m, 2H), 7.83 (d, 1H), 8.51 (d, 1H).

### Step P1.4: Preparation of 2-[6-(3,5-difluorophenyl)-3-ethylsulfonyl-2-pyridyl]-6-(trifluoromethyl)pyrazolo[4,3-c]pyridine (compound P1)

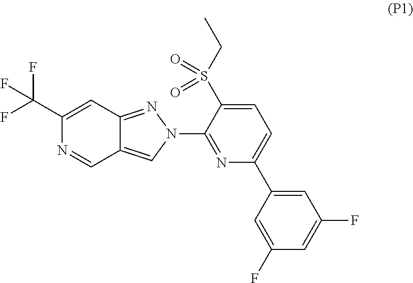 [SMILES: CCS(=O)(=O)c1ccc(-c2cc(F)cc(F)c2)nc1-n1cc2cnc(C(F)(F)F)cc2n1]

To a solution of 2-chloro-6-(3,5-difluorophenyl)-3-ethylsulfonyl-pyridine (270 mg, 0.85 mmol) and 6-(trifluoromethyl)-2H-pyrazolo[4,3-c]pyridine (159 mg, 0.85 mmol) in N,N-dimethylformamide (6.5 ml) under argon was added potassium carbonate (352 mg, 2.55 mmol) and the mixture was heated at 100° C. overnight. The reaction mixture was concentrated in vacuo and the residue purified over silica by flash column chromatography (0-30% ethyl acetate gradient in cyclohexane) and by C₁₈ reversed-phase column chromatography (acetonitrile gradient in water) to afford 2-[6-(3,5-difluorophenyl)-3-ethylsulfonyl-2-pyridyl]-6-(trifluoromethyl)pyrazolo[4,3-c]pyridine (compound P1, minor isomer) as a solid. LCMS (method 2): 469 (M+H)⁺, retention time 1.93 min. ¹H-NMR (CDCl₃, ppm) δ 1.48 (t, 3H), 3.99 (q, 2H), 7.03 (br t, 1H), 7.64-7.71 (m, 2H), 8.06-8.11 (m, 2H), 8.72 (d, 1H), 9.03 (s, 1H), 9.46 (s, 1H). The major product of the reaction is the regioisomer 1-[6-(3,5-difluorophenyl)-3-ethylsulfonyl-2-pyridyl]-6-(trifluoromethyl)pyrazolo[4,3-c]pyridine obtained as a solid. LCMS (method 2): 469 (M+H)⁺, retention time 1.84 min.

### Example P2: Preparation of 3-chloro-2-[3-ethylsulfonyl-6-(1,2,4-triazol-1-yl)-2-pyridyl]-6-(trifluoromethyl)pyrazolo[4,3-c]pyridine (Compound P2) and 2-[3-ethylsulfonyl-6-(1,2,4-triazol-1-yl)-2-pyridyl]-6-(trifluoromethyl)pyrazolo[4,3-c]pyridine (Compound P3)

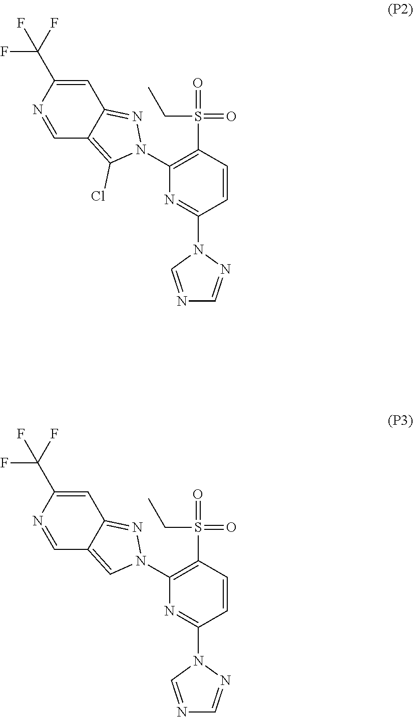 [SMILES: CCS(=O)(=O)c1ccc(-n2cncn2)nc1-n1nc2cc(C(F)(F)F)ncc2c1Cl ; CCS(=O)(=O)c1ccc(-n2cncn2)nc1-n1cc2cnc(C(F)(F)F)cc2n1]

### Step P2.01: Preparation of 2-bromo-6-chloro-3-ethylsulfanyl-pyridine

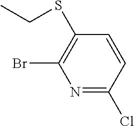

A solution of (ethyldisulfanyl)ethane (48 mmol, 5.9 g, 5.9 mL) and tert-butyl nitrite (36 mmol, 3.7 g, 4.3 mL) in 1,2-dichloroethane (100 ml) was heated at 40° C. To this solution was added, slowly over 60 min, a solution of 2-bromo-6-chloro-pyridin-3-amine (commercially available (CAS 1050501-88-6), 24 mmol, 5.0 g) in 1,2-dichloroethane (100 ml). The reaction was stirred at 40° C. for 3 h. Then, the reaction was diluted with water (10 ml), extracted with ethyl acetate (3×30 ml). Combined organic layer was dried over sodium sulfate, filtered and concentrated under reduced pressure. The crude product was purified by combiflash (silica gel, 20% EA-cyclohexane) to afford 2-bromo-6-chloro-3-ethylsulfanyl-pyridine (16 mmol, 4.0 g). ¹H-NMR (CDCl₃, ppm) δ 7.42 (d, 1H), 7.25 (d, 1H), 2.95 (q, 2H), 1.38 (t, 3H).

### Step P2.02: Preparation of 2-bromo-6-chloro-3-ethylsulfonyl-pyridine

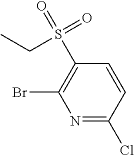

To a solution of 2-bromo-6-chloro-3-ethylsulfanyl-pyridine (7.9 mmol, 2.0 g) in dichloromethane (30 mL) was added 3-chlorobenzenecarboperoxoic acid (17 mmol, 4.3 g). The reaction was stirred, overnight, at ambient temperature. The reaction was cooled and quenched with a aqueous solution sodium hydroxide (1N, 20 ml), extracted with DCM (3×20 ml). Combined organic layer was dried over sodium sulfate, filtered and concentrated under reduced pressure. The crude product was purified by combiflash (silica gel, 30% EA-cyclohexane) to afford 2-bromo-6-chloro-3-ethylsulfonyl-pyridine (6.7 mmol, 1.9 g). ¹H-NMR (CDCl₃, ppm) δ 8.36 (d, 1H), 7.53 (d, 1H), 3.53 (q, 2H), 1.36 (t, 3H).

### Step P2.03: Preparation of (6-chloro-3-ethylsulfonyl-2-pyridyl)hydrazine

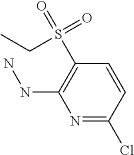

To a solution of 2-bromo-6-chloro-3-ethylsulfonyl-pyridine (1.757 mmol, 0.5 g) in ethanol (5.0 mL) was added hydrazine hydrate (8.785 mmol, 0.4398 g, 0.43 mL). The reaction was stirred under nitrogen atmosphere at ambient temperature for 1.2 h. The reaction was quenched with sodium bicarbonate solution (10 ml), extracted with ethyl acetate (3×30 ml). Combined organic layer was dried over sodium sulfate, filtered and concentrated under reduced pressure. The crude product was purified by combiflash (silica gel, 40% EA-cyclohexane) to afford (6-chloro-3-ethylsulfonyl-2-pyridyl)hydrazine (1.188 mmol, 0.28 g). ¹H-NMR (CDCl₃, ppm) δ 7.87 (d, 1H), 6.80 (d, 1H), 3.60-3.31 (sb, 1H), 3.14 (q, 3H), 1.28 (t, 3H).

### Step P2.04: Preparation of 4-[2-(6-chloro-3-ethylsulfonyl-2-pyridyl)hydrazino]-6-(trifluoromethyl)pyridine-3-carboxylic acid

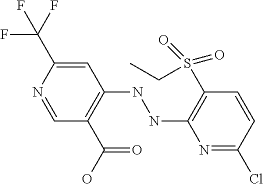 [SMILES: CCS(=O)(=O)c1ccc(Cl)nc1NNc1cc(C(F)(F)F)ncc1C(=O)O]

To 4-chloro-6-(trifluoromethyl)pyridine-3-carboxylic acid (10 mmol, 2.3 g) was added (6-chloro-3-ethylsulfonyl-2-pyridyl)hydrazine (15 mmol, 3.6 g) in pentan-1-ol (20 mL) and the reaction was heated at 110° C. for 5 h. The reaction was concentrated under reduced pressure to remove all pentanol. The residue was co-evaporated with toluene to remove any pentanol. Then, the reaction was diluted with a mixture of water (20 ml) and brine (10 ml), extracted with ethyl acetate (3×20 ml). The combined organic layer was dried over sodium sulfate, filtered and concentrated under reduced pressure. The solid residue obtained was triturated with cyclohexane to afford 4-[2-(6-chloro-3-ethylsulfonyl-2-pyridyl)hydrazino]-6-(trifluoromethyl)pyridine-3-carboxylic acid (5.9 mmol, 2.5 g). This compound was used in the next step without any extra purification.

### Step P2.05: Preparation of 3-chloro-2-(6-chloro-3-ethylsulfanyl-2-pyridyl)-6-(trifluoromethyl) pyrazolo[4,3-c]pyridine

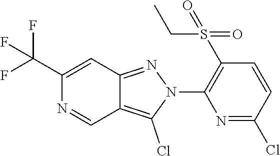 [SMILES: CCS(=O)(=O)c1ccc(Cl)nc1-n1nc2cc(C(F)(F)F)ncc2c1Cl]

A solution of 4-[2-(6-chloro-3-ethylsulfonyl-2-pyridyl)hydrazino]-6-(trifluoromethyl)pyridine-3-carboxylic acid (0.4708 mmol, 0.2 g) in phosphorus oxychloride (2.0 mL) was refluxed at 110° C. for 1.5 h. The reaction was poured into ice (500 g) and was added of solid sodium bicarbonate. The aqueous layer was extracted with dichloromethane (3×200 ml). The combined organic layer was dried over sodium sulfate, filtered and concentrated under reduced pressure. The residue was purified by trituration with cyclohexane to afford 3-chloro-2-(6-chloro-3-ethylsulfonyl-2-pyridyl)-6-(trifluoromethyl)pyrazolo[4,3-c]pyridine (0.235 mmol, 0.1 g). ¹H-NMR (CDCl₃, ppm) δ 9.29 (s, 1H), 8.55 (d, 1H), 7.97 (s, 1H), 7.86 (d, 1H), 3.57 (q, 2H), 1.35 (t, 3H).

### Step P2.06: Preparation of 3-chloro-2-[3-ethylsulfonyl-6-(1,2,4-triazol-1-yl)-2-pyridyl]-6-(trifluoromethyl)pyrazolo[4,3-c]pyridine (compound P2)

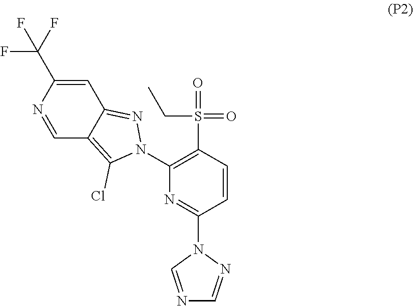 [SMILES: CCS(=O)(=O)c1ccc(-n2cncn2)nc1-n1nc2cc(C(F)(F)F)ncc2c1Cl]

To a solution of 3-chloro-2-(6-chloro-3-ethylsulfonyl-2-pyridyl)-6-(trifluoromethyl)pyrazolo[4,3-c]pyridine (0.823 mmol, 0.35 g) in N,N-dimethylformamide (8.0 mL) was added dipotassium carbonic acid 0.823 mmol, 0.1154 g) followed by 1H-1,2,4-triazole (0.823 mmol, 0.0569 g). The mixture was stirred at room temperature for 3 hrs. The reaction was diluted with water (10 ml), extracted with ethyl acetate (3×20 ml). Combined organic layer was dried over sodium sulfate, filtered and concentrated under reduced pressure. The residue was purified by combiflash (silica gel, 40% EA-cyclohexane) to afford 3-chloro-2-[3-ethylsulfonyl-6-(1,2,4-triazol-1-yl)-2-pyridyl]-6-(trifluoromethyl)pyrazolo[4,3-c]pyridine (0.371 mmol, 0.17 g). ¹H-NMR (CDCl₃, ppm) δ 9.33 (s, 1H), 9.13 (s, 1H), 8.79 (d, 1H), 8.41 (d, 1H), 8.22 (s, 1H), 8.02 (s, 1H), 3.60 (q, 2H), 1.36 (t, 3H).

### Step P2.07: Preparation of 2-[3-ethylsulfonyl-6-(1,2,4-triazol-1-yl)-2-pyridyl]-6-(trifluoromethyl)pyrazolo[4,3-c]pyridine (compound P3)

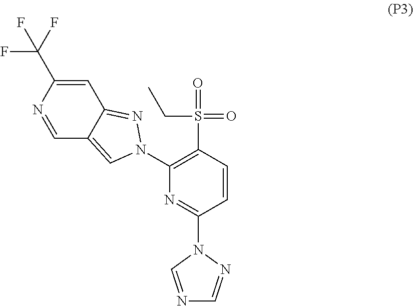 [SMILES: CCS(=O)(=O)c1ccc(-n2cncn2)nc1-n1cc2cnc(C(F)(F)F)cc2n1]

To a solution of 3-chloro-2-[3-ethylsulfonyl-6-(1,2,4-triazol-1-yl)-2-pyridyl]-6-(trifluoromethyl) pyrazolo[4,3-c]pyridine (0.218 mmol, 0.1 g) in acetic acid (6.0 mL) was added zinc (0.874 mmol, 0.05715 g). The reaction was heated at 60° C. for 2-3 h and then diluted with water (10 ml) and extracted with ethyl acetate (3×20 ml). Combined organic layer was dried over sodium sulfate, filtered and concentrated under reduced pressure. The crude product was purified by combiflash (silica gel, 50% EA-cyclohexane) to afford 2-[3-ethylsulfonyl-6-(1,2,4-triazol-1-yl)-2-pyridyl]-6-(trifluoromethyl) pyrazolo[4,3-c]pyridine (0.0827 mmol, 0.035 g). ¹H-NMR (CDCl₃, ppm) δ 9.45 (s, 1H), 9.18 (s, 1H), 8.93 (s, 1H), 8.83 (d, 1H), 8.32 (d, 1H), 8.22 (s, 1H), 8.08 (s, 1H), 3.94 (m, 2H), 1.45 (m, 3H).

### Example P3: Preparation of 3-chloro-2-[3-ethylsulfonyl-6-(3-pyridyl)-2-pyridyl]-6-(trifluoromethyl)pyrazolo[4,3-c]pyridine (compound P17) and 2-[3-ethylsulfonyl-6-(3-pyridyl)-2-pyridyl]-6-(trifluoromethyl)pyrazolo[4,3-c]pyridine (compound P12)

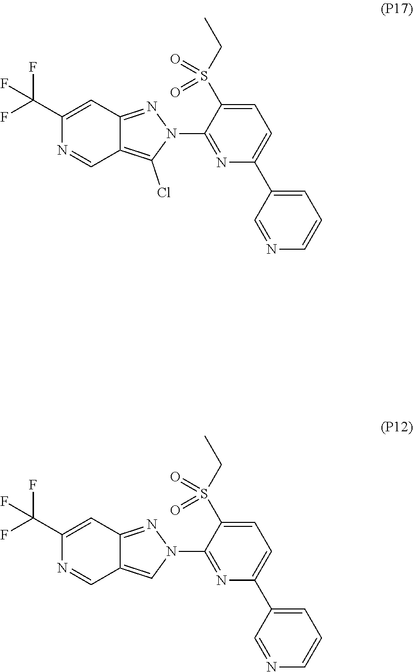 [SMILES: CCS(=O)(=O)c1ccc(-c2cccnc2)nc1-n1nc2cc(C(F)(F)F)ncc2c1Cl ; CCS(=O)(=O)c1ccc(-c2cccnc2)nc1-n1cc2cnc(C(F)(F)F)cc2n1]

### Step P3.1: Preparation of 3-chloro-2-[3-ethylsulfonyl-6-(3-pyridyl)-2-pyridyl]-6-(trifluoromethyl) pyrazolo[4,3-c]pyridine (compound P17)

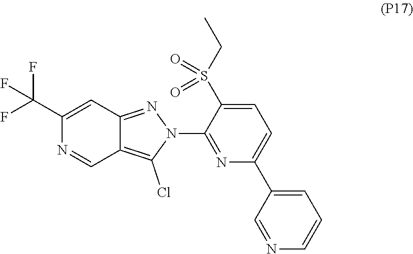 [SMILES: CCS(=O)(=O)c1ccc(-c2cccnc2)nc1-n1nc2cc(C(F)(F)F)ncc2c1Cl]

To a solution of 3-chloro-2-(6-chloro-3-ethylsulfonyl-2-pyridyl)-6-(trifluoromethyl)pyrazolo[4,3-c]pyridine (200 mg, 0.470 mmol) in 1,4-dioxane (10 ml) and water (1 ml) was added tricyclohexylphosphane (26.4 mg, 0.094 mmol), tripotassium phosphate (250 mg, 1.176 mmol) and 3-pyridylboronic acid (57.8 mg, 0.470 mmol). The reaction mixture was degassed with nitrogen for 20 min before adding tris(dibenzylideneacetone)dipalladium(O) (44.4 mg, 0.047 mmol). Degassing was continued for additional 20 min before heating the reaction mass for 3 hours at 100° C. After cooling, the reaction mixture was poured into cold water, the mixture was diluted with ethyl acetate and filtered through diatomaceous earth (Hyflo). The filter was thoroughly washed with ethyl acetate. The layers were separated, and the aqueous layer was extracted with additional ethyl acetate. The combined organic layers were washed with brine, dried over sodium sulfate, filtered, and concentrated under vacuum. The residue was purified over silica by flash column chromatography (80% ethyl acetate in cyclohexane) to afford 3-chloro-2-[3-ethylsulfonyl-6-(3-pyridyl)-2-pyridyl]-6-(trifluoromethyl) pyrazolo[4,3-c]pyridine (compound P17) as a solid, mp 194-196° C. LCMS (method 4): 468/470 (M+H)⁺, retention time 1.01 min.

### Step P3.2: Preparation of 2-[3-ethylsulfonyl-6-(3-pyridyl)-2-pyridyl]-6-(trifluoromethyl)pyrazolo[4,3-c]pyridine (compound P12)

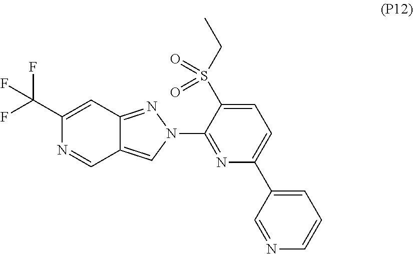 [SMILES: CCS(=O)(=O)c1ccc(-c2cccnc2)nc1-n1cc2cnc(C(F)(F)F)cc2n1]

To a solution of 3-chloro-2-[3-ethylsulfonyl-6-(3-pyridyl)-2-pyridyl]-6-(trifluoromethyl)pyrazolo[4,3-c]pyridine (60.0 mg, 0.128 mmol) in acetic acid (8.0 ml) and trifluroacetic acid (1 ml) was added zinc (33.6 mg, 0.513 mmol). The reaction was heated at 80° C. for three hours. After cooling, the solvents were removed under vacuum, the residue was diluted with water (10 ml), the aqueous phase was saturated with potassium carbonate, and extracted with ethyl acetate (3×15 ml). The combined organic layers were dried over sodium sulfate, filtered and concentrated under reduced pressure. The crude material was purified by preparative TLC (Rf=0.35 with 50% ethyl acetate in cyclohexane) to afford 2-[3-ethylsulfonyl-6-(3-pyridyl)-2-pyridyl]-6-(trifluoromethyl)pyrazolo[4,3-c]pyridine (compound P12) as a solid, mp 246-248° C. LCMS (method 4): 434 (M+H)⁺, retention time 0.97 min.

### Example P4: Preparation of 5-ethylsulfonyl-6-[6-(trifluoromethyl)pyrazolo[4,3-c]pyridin-2-yl]pyridin-2-amine (compound P16) and 3-chloro-5-ethylsulfonyl-6-[6-(trifluoromethyl)pyrazolo[4,3-c]pyridin-2-yl]pyridin-2-amine (compound P19)

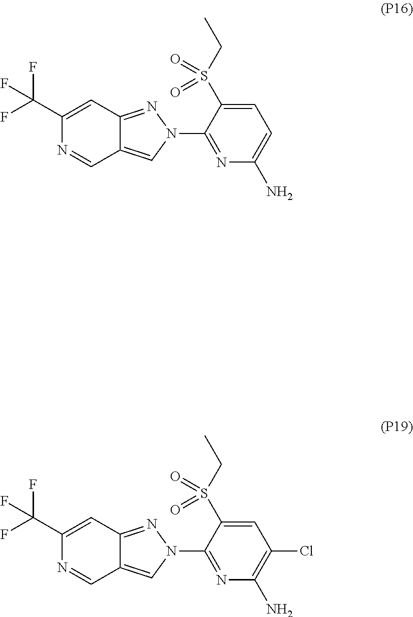 [SMILES: CCS(=O)(=O)c1ccc(N)nc1-n1cc2cnc(C(F)(F)F)cc2n1 ; CCS(=O)(=O)c1cc(Cl)c(N)nc1-n1cc2cnc(C(F)(F)F)cc2n1]

### Step P4.1: Preparation of 6-[3-chloro-6-(trifluoromethyl)pyrazolo[4,3-c]pyridin-2-yl]-5-ethylsulfonyl-pyridin-2-amine (compound P14)

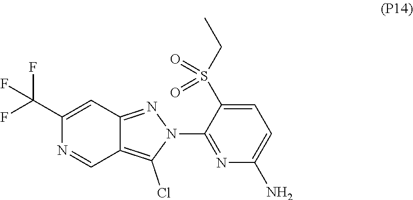 [SMILES: CCS(=O)(=O)c1ccc(N)nc1-n1nc2cc(C(F)(F)F)ncc2c1Cl]

To a solution of 3-chloro-2-(6-chloro-3-ethylsulfonyl-2-pyridyl)-6-(trifluoromethyl)pyrazolo[4,3-c]pyridine (50.0 mg, 0.118 mmol) in tetrahydrofuran (2 ml) under nitrogen was added an aqueous 25% wt ammonium hydroxide solution (0.072 ml, 0.470 mmol). The reaction mixture was stirred overnight at room temperature, then poured into water. The aqueous phase was extracted three times with ethyl acetate, the combined organic layers were washed with brine, dried over sodium sulfate and evaporated under reduced pressure to give the crude product 6-[3-chloro-6-(trifluoromethyl) pyrazolo[4,3-c]pyridin-2-yl]-5-ethylsulfonyl-pyridin-2-amine (compound P14) as a solid, mp 310-312° C. This material was used without further purification. LCMS (method 3): 406/408 (M+H)⁺, retention time 0.89 min.

### Step P4.2: Preparation of 5-ethylsulfonyl-6-[6-(trifluoromethyl)pyrazolo[4,3-c]pyridin-2-yl]pyridin-2-amine (compound P16)

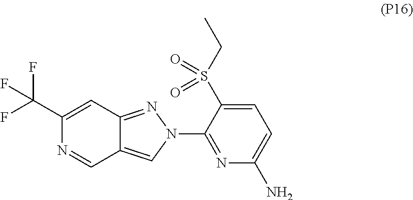 [SMILES: CCS(=O)(=O)c1ccc(N)nc1-n1cc2cnc(C(F)(F)F)cc2n1]

To a solution of 6-[3-chloro-6-(trifluoromethyl)pyrazolo[4,3-c]pyridin-2-yl]-5-ethylsulfonyl-pyridin-2-amine (301 mg, 0.742 mmol) in acetic acid (14 ml) and trifluoroacetic acid (0.8 ml) was added zinc (194 mg, 2.98 mmol). The reaction mixture was heated at 60° C. for 4h. After cooling, the reaction mixture was quenched with a saturated sodium bicarbonate solution, and the aqueous phase was extracted with ethyl acetate. The combined organic layers were washed with water, brine, dried with sodium sulfate and concentrated under reduced pressure. The residue was purified over silica by flash column chromatography using ethyl acetate/hexane (6:4) to afford 5-ethylsulfonyl-6-[6-(trifluoromethyl) pyrazolo[4,3-c]pyridin-2-yl]pyridin-2-amine (compound P16) as a solid, mp 224-226° C. LCMS (method 3): 372 (M+H)⁺, retention time 0.86 min.

### Step P4.3: Preparation of 3-chloro-5-ethylsulfonyl-6-[6-(trifluoromethyl)pyrazolo[4,3-c]pyridin-2-yl]pyridin-2-amine (compound P19)

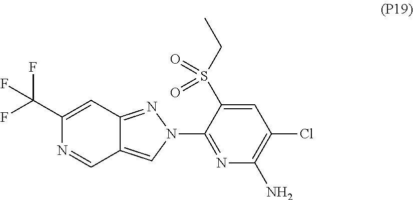 [SMILES: CCS(=O)(=O)c1cc(Cl)c(N)nc1-n1cc2cnc(C(F)(F)F)cc2n1]

To a solution of 5-ethylsulfonyl-6-[6-(trifluoromethyl)pyrazolo[4,3-c]pyridin-2-yl]pyridin-2-amine (150 mg, 0.404 mmol) in N,N-dimethylformamide (4 ml) under nitrogen was added N-chlorosuccinimide (110 mg, 0.808 mmol). The reaction mixture was stirred overnight at room temperature. The reaction mixture was quenched with water and brine, and the aqueous phase was extracted with ethyl acetate. The combined organic layers were washed with water, brine, dried over sodium sulfate and concentrated under reduced pressure. The crude material was purified over silica by flash column chromatography to afford 3-chloro-5-ethylsulfonyl-6-[6-(trifluoromethyl)pyrazolo[4,3-c]pyridin-2-yl]pyridin-2-amine (compound P19) as a pale yellow solid, mp 234-236° C. LCMS (method 3): 406/408 (M+H)⁺, retention time 1.39 min.

### Example P5: Preparation of 2-[3-ethylsulfonyl-6-(1,2,4-triazol-1-yl)-2-pyridyl]-3-methyl-6-(trifluoromethyl)pyrazolo[4,3-c]pyridine (compound P22)

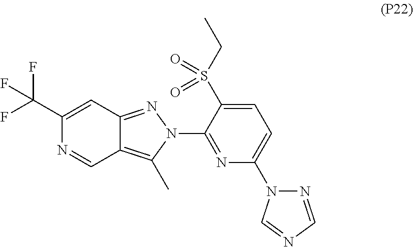 [SMILES: CCS(=O)(=O)c1ccc(-n2cncn2)nc1-n1nc2cc(C(F)(F)F)ncc2c1C]

To a solution of 3-chloro-2-[3-ethylsulfonyl-6-(1,2,4-triazol-1-yl)-2-pyridyl]-6-(trifluoromethyl) pyrazolo[4,3-c]pyridine (100 mg, 0.218 mmol) and 2,4,6-trimethyl-1,3,5,2,4,6-trioxatriborinane (32.9 mg, 0.04 ml, 0.262 mmol) in 1,4-dioxane (3 ml) was added potassium bicarbonate (91.9 mg, 0.655 mmol). The reaction mixture was degassed for 10 min with nitrogen before adding tetrakis(triphenyl-phosphine)palladium (25.2 mg, 0.022 mmol). The reaction mixture was heated at 100° C. overnight. After cooling, the reaction was diluted with water (10 ml), and the aqueous phase was extracted with ethyl acetate (3×20 ml). The combined organic layer were dried over sodium sulfate and concentrated under reduced pressure. The crude product was purified over silica by flash column chromatography (ethyl acetate 30% in cyclohexane) to afford 2-[3-ethylsulfonyl-6-(1,2,4-triazol-1-yl)-2-pyridyl]-3-methyl-6-(trifluoromethyl)pyrazolo[4,3-c]pyridine (compound P22) as a solid, mp 233-235° C. LCMS (method 3): 438 (M+H)⁺, retention time 1.38 min.

### Example P6: Preparation of 2-(5-chloro-3-ethylsulfonyl-6-methoxy-2-pyridyl)-6-(trifluoromethyl)pyrazolo[4,3-c]pyridine (compound P24)

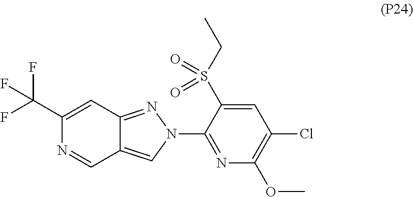 [SMILES: CCS(=O)(=O)c1cc(Cl)c(OC)nc1-n1cc2cnc(C(F)(F)F)cc2n1]

### Step P6.1: Preparation of 6-[3-chloro-6-(trifluoromethyl)pyrazolo[4,3-c]pyridin-2-yl]-5-ethylsulfonyl-pyridin-2-ol

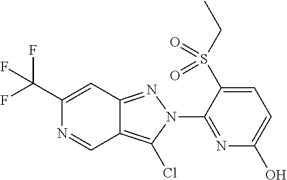 [SMILES: CCS(=O)(=O)c1ccc(O)nc1-n1nc2cc(C(F)(F)F)ncc2c1Cl]

To s solution of 3-chloro-2-(6-chloro-3-ethylsulfonyl-2-pyridyl)-6-(trifluoromethyl)pyrazolo[4,3-c]pyridine (5.0 g, 12 mmol) in tetrahydrofuran (50 ml) was added a solution of sodium hydroxide (1.9 g, 47 mmol) in water (10 ml). The reaction mixture was stirred at 50° C. for 6 h. After cooling, the reaction mixture was acidified with a 1N hydrochloric acid solution, and the aqueous layer was extracted with ethyl acetate (3×20 ml). The combined organic layers were dried over sodium sulfate, filtered and concentrated under reduced pressure. The crude product was purified by trituration using cyclohexane to afford 6-[3-chloro-6-(trifluoromethyl)pyrazolo[4,3-c]pyridin-2-yl]-5-ethylsulfonyl-pyridin-2-ol. This material was used without further purification. LCMS (method 3): 407/409 (M+H)⁺, retention time 1.37 min.

### Step P6.2: Preparation of 3-chloro-6-[3-chloro-6-(trifluoromethyl)pyrazolo[4,3-c]pyridin-2-yl]-5-ethylsulfonyl-pyridin-2-ol

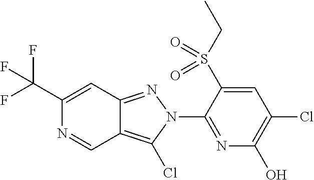 [SMILES: CCS(=O)(=O)c1cc(Cl)c(O)nc1-n1nc2cc(C(F)(F)F)ncc2c1Cl]

To a solution of 6-[3-chloro-6-(trifluoromethyl)pyrazolo[4,3-c]pyridin-2-yl]-5-ethylsulfonyl-pyridin-2-ol (1.40 g, 3.44 mmol) in acetonitrile (20 ml) was added N-chlorosuccinimide (0.83 g, 6.21 mmol). The reaction mixture was heated at 55° C. for 3h. After cooling down to room temperature, the reaction was quenched with water (50 ml), and the aqueous phase was extracted with ethyl acetate (3×50 ml). The combined organic layers were dried over sodium sulfate, filtered and concentrated under reduced pressure. The crude product was purified over silica by flash column chromatography (pure ethyl acetate) to afford 3-chloro-6-[3-chloro-6-(trifluoromethyl)pyrazolo[4,3-c]pyridin-2-yl]-5-ethylsulfonyl-pyridin-2-ol. LCMS (method 3): 441/443 (M+H)⁺, retention time 1.39 min.

### Step P6.3: Preparation of 3-chloro-2-(5-chloro-3-ethylsulfonyl-6-methoxy-2-pyridyl)-6-(trifluoromethyl)pyrazolo[4,3-c]pyridine

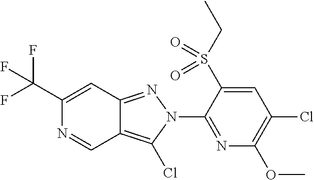 [SMILES: CCS(=O)(=O)c1cc(Cl)c(OC)nc1-n1nc2cc(C(F)(F)F)ncc2c1Cl]

To a solution of sodium hydride (21.8 mg, 0.544 mmol) in N,N-dimethylformamide (2 ml), cooled at 0° C. and under nitrogen, was added dropwise a solution of 3-chloro-6-[3-chloro-6-(trifluoromethyl) pyrazolo[4,3-c]pyridin-2-yl]-5-ethylsulfonyl-pyridin-2-ol (200 mg, 0.453 mmol) in N,N-dimethylformamide (2 ml). The reaction mixture was stirred for 40 min at 0° C. Iodomethane (128 mg, 58 μI, 0.907 mmol) was then added, the cooling bath was removed, and the reaction was stirred at room temperature overnight. The reaction mixture was quenched carefully at 0° C. with water (20 ml), and the aqueous phase was extracted with ethyl acetate (3×20 ml). The combined organic layers were dried over sodium sulfate, filtered and concentrated under reduced pressure. The crude product was purified over silica by flash column chromatography (20% ethyl acetate in cyclohexane) to afford 3-chloro-2-(5-chloro-3-ethylsulfonyl-6-methoxy-2-pyridyl)-6-(trifluoromethyl)pyrazolo[4,3-c]pyridine. LCMS (method 3): 455/457 (M+H)⁺, retention time 1.56 min.

The regioisomeric N-methylation product 3-chloro-6-[3-chloro-6-(trifluoromethyl)pyrazolo[4,3-c]pyridin-2-yl]-5-ethylsulfonyl-1-methyl-pyridin-2-one was also isolated from this reaction, LCMS (method 3): 455/457 (M+H)⁺, retention time 1.44 min.

### Step P6.4: Preparation of 2-(5-chloro-3-ethylsulfonyl-6-methoxy-2-pyridyl)-6-(trifluoromethyl)pyrazolo[4,3-c]pyridine (compound P24)

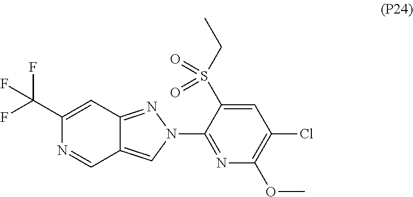 [SMILES: CCS(=O)(=O)c1cc(Cl)c(OC)nc1-n1cc2cnc(C(F)(F)F)cc2n1]

To a solution of 3-chloro-2-(5-chloro-3-ethylsulfonyl-6-methoxy-2-pyridyl)-6-(trifluoromethyl)pyrazolo [4,3-c]pyridine (120 mg, 0.264 mmol) in acetic acid (1.5 ml) and trifluoroacetic acid (0.1 ml) was added zinc (51.7 mg, 0.791 mmol). The reaction mixture was heated at 60° C. for 1.5h. After cooling to room temperature, the reaction mixture was slowly quenched by addition of a saturated sodium bicarbonate aqueous solution (10 ml), and the aqueous phase was extracted with ethyl acetate (3×20 ml). The combined organic layers were dried over sodium sulfate, filtered and concentrated under reduced pressure. The crude product was purified over silica by flash column chromatography (20% ethyl acetate in cyclohexane) to afford 2-(5-chloro-3-ethylsulfonyl-6-methoxy-2-pyridyl)-6-(trifluoro-methyl)pyrazolo[4,3-c]pyridine (compound P24) as a solid, mp 226-228° C. LCMS (method 3): 421/423 (M+H)⁺, retention time 1.51 min.

### Example P7: Preparation of 1-[6-[3-chloro-6-(trifluoromethyl)pyrazolo[4,3-c]pyridin-2-yl]-5-ethylsulfonyl-2-pyridyl]pyrazole-4-carbonitrile (compound P10)

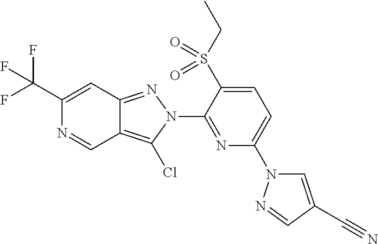 [SMILES: CCS(=O)(=O)c1ccc(-n2cc(C#N)cn2)nc1-n1nc2cc(C(F)(F)F)ncc2c1Cl]

To a solution of 3-chloro-2-(6-chloro-3-ethylsulfonyl-2-pyridyl)-6-(trifluoromethyl)pyrazolo[4,3-c]pyridine (425 mg, 1 mmol) in N,N-dimethylformamide (4 mL) was added dipotassium carbonic acid (140 mg, 1 mmol), followed by 1H-pyrazole-4-carbonitrile (93 mg, 1 mmol). The mixture was stirred at room temperature for 3 hrs. The reaction was diluted with ice water (20 ml), extracted with ethyl acetate (2×40 ml), the combined organic layers washed with water and brine, dried over sodium sulfate, filtered and concentrated under reduced pressure. The crude product was purified by combiflash (silica gel, 40% EA-cyclohexane) to afford 1-[6-[3-chloro-6-(trifluoromethyl)pyrazolo[4,3-c]pyridin-2-yl]-5-ethylsulfonyl-2-pyridyl]pyrazole-4-carbonitrile (compound P10) as a solid, mp 251-253° C. LCMS (method 3): 482/484 (M+H)⁺, retention time 1.50 min.

### Example P8: Preparation of 3-chloro-2-(6-chloro-3-ethylsulfonyl-2-pyridyl)-6-(trifluoromethyl) pyrazolo[4,3-c]pyridazine

### Step P8.1: Preparation of 4-iodo-6-(trifluoromethyl)pyridazine-3-carboxylic acid

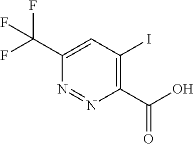 [SMILES: O=C(O)c1nnc(C(F)(F)F)cc1I]

A solution of ethyl 4-iodo-6-(trifluoromethyl)pyridazine-3-carboxylate (preparation according to WO 2016020286) (1.10 g, 3.18 mmol) in a tetrahydrofuran/water mixture (3:1 v/v, 25 ml) was treated with lithium hydroxide monohydrate (140 mg, 3.34 mmol) at 0° C. The reaction mixture was stirred at 0° C. for 40 minutes, diluted with tert-butyl methyl ether and acidified by careful addition of aqueous hydrochloric acid at 0° C. The product was extracted with ethyl acetate (4×50 ml), the combined organic phases dried over sodium sulfate, filtered and concentrated under reduced pressure to afford the crude product 4-iodo-6-(trifluoromethyl)pyridazine-3-carboxylic acid as a solid. This material was used without further purification. ¹H-NMR (d₆-DMSO, ppm) δ 8.94 (s, 1H), 14.8 (br s, 1H). LCMS (method 1): 319 (M+H)⁺, retention time 0.32 min.

### Step P8.2: Preparation of 4-[2-(6-chloro-3-ethylsulfonyl-2-pyridyl)hydrazino]-6-(trifluoromethyl) pyridazine-3-carboxylic acid

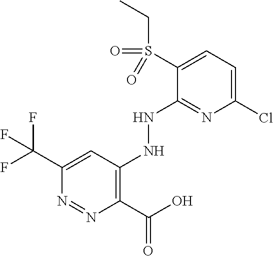 [SMILES: CCS(=O)(=O)c1ccc(Cl)nc1NNc1cc(C(F)(F)F)nnc1C(=O)O]

To 4-iodo-6-(trifluoromethyl)pyridazine-3-carboxylic acid (550 mg, 1.73 mmol) in acetonitrile (5 ml) was added cesium carbonate (564 mg, 1.73 mmol), the reaction mixture was stirred at 30-35° C. for 45 minutes, then concentrated to dryness under reduced pressure. The residue was diluted with pentan-1-01 (5 mL) and (6-chloro-3-ethylsulfonyl-2-pyridyl)hydrazine (408 mg, 1.73 mmol) added. The reaction mixture was heated at 60° C. for 4 h, then concentrated under reduced pressure. The residue was diluted with water and ethyl acetate, acidified by careful addition of aqueous hydrochloric acid at 0° C., and the product extracted with ethyl acetate (5×20 ml). The combined organic phases were washed with brine, dried over sodium sulfate, filtered and concentrated under reduced pressure. The residue was purified by combiflash (silica gel, gradient 0-2% methanol in dichloromethane) to afford 4-[2-(6-chloro-3-ethylsulfonyl-2-pyridyl)hydrazino]-6-(trifluoromethyl)pyridazine-3-carboxylic acid as a solid. LCMS (method 1): 426/428 (M+H)⁺, retention time 0.80 min.

### Step P8.3: Preparation of 3-chloro-2-(6-chloro-3-ethylsulfonyl-2-pyridyl)-6-(trifluoromethyl) pyrazolo[4,3-c]pyridazine

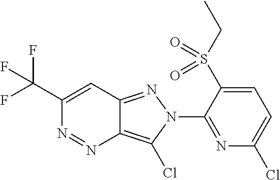 [SMILES: CCS(=O)(=O)c1ccc(Cl)nc1-n1nc2cc(C(F)(F)F)nnc2c1Cl]

A solution of 4-[2-(6-chloro-3-ethylsulfonyl-2-pyridyl)hydrazino]-6-(trifluoromethyl)pyridazine-3-carboxylic acid (150 mg, 0.352 mmol) in phosphorus oxychloride (3 ml) was heated to 80° C. for one hour. The reaction was poured into ice (20 g) and the mixture neutralized by careful addition of solid sodium carbonate in portions. The aqueous layer was extracted with dichloromethane (5×20 ml), the combined organic layers dried over sodium sulfate, filtered and concentrated under reduced pressure. The residue was purified by combiflash (silica gel, 1% methanol in dichloromethane) to afford 3-chloro-2-(6-chloro-3-ethylsulfonyl-2-pyridyl)-6-(trifluoromethyl) pyrazolo[4,3-c]pyridazine as a solid. LCMS (method 1): 426/428 (M+H)⁺, retention time 1.02 min.

TABLE P
Examples of compounds of formula (I)

| | | LCMS | | | |
|---|---|---|---|---|---|
| Compound | | Rₜ | [M + H]⁺ | | |
| No. | Structures | (min) | (measured) | Method | MP (° C.) |
| P1 | 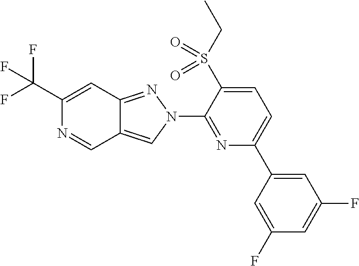 [SMILES: CCS(=O)(=O)c1ccc(-c2cc(F)cc(F)c2)nc1-n1cc2cnc(C(F)(F)F)cc2n1] | 1.93 | 469 | 2 | 207-209 |
| P2 | 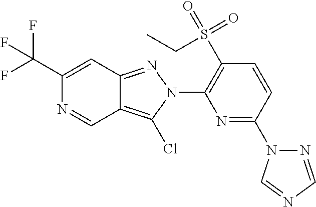 [SMILES: CCS(=O)(=O)c1ccc(-n2cncn2)nc1-n1nc2cc(C(F)(F)F)ncc2c1Cl] | 1.43 | 458/460 | 3 | 185-187 |
| P3 | 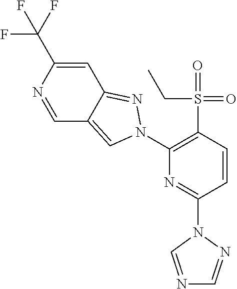 [SMILES: CCS(=O)(=O)c1ccc(-n2cncn2)nc1-n1cc2cnc(C(F)(F)F)cc2n1] | 1.39 | 424 | 3 | 273-275 |
| P4 | 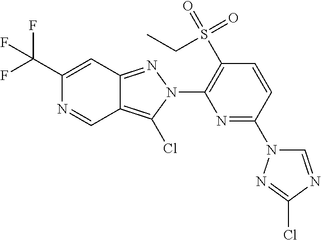 [SMILES: CCS(=O)(=O)c1ccc(-n2cnc(Cl)n2)nc1-n1nc2cc(C(F)(F)F)ncc2c1Cl] | 1.52 | 492/494 | 3 | 197-199 |
| P5 | 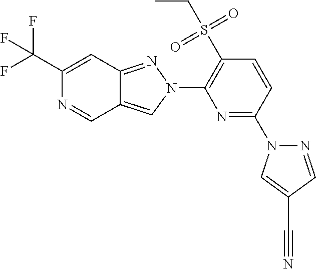 [SMILES: CCS(=O)(=O)c1ccc(-n2cc(C#N)cn2)nc1-n1cc2cnc(C(F)(F)F)cc2n1] | 1.48 | 448 | 3 | 300-302 |
| P6 | 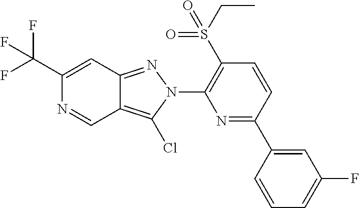 [SMILES: CCS(=O)(=O)c1ccc(-c2cccc(F)c2)nc1-n1nc2cc(C(F)(F)F)ncc2c1Cl] | 1.62 | 485/487 | 3 | 280-282 |
| P7 | 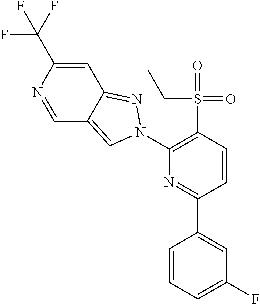 [SMILES: CCS(=O)(=O)c1ccc(-c2cccc(F)c2)nc1-n1cc2cnc(C(F)(F)F)cc2n1] | 1.57 | 451 | 3 | 205-207 |
| P8 | 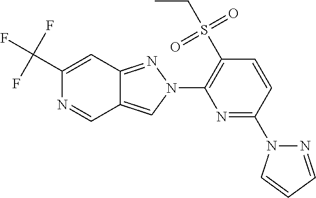 [SMILES: CCS(=O)(=O)c1ccc(-n2cccn2)nc1-n1cc2cnc(C(F)(F)F)cc2n1] | 1.49 | 423 | 3 | 278-280 |
| P9 | 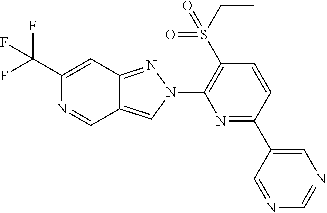 [SMILES: CCS(=O)(=O)c1ccc(-c2cncnc2)nc1-n1cc2cnc(C(F)(F)F)cc2n1] | 1.36 | 435 | 3 | 266-268 |
| P10 | 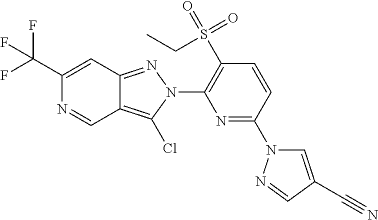 [SMILES: CCS(=O)(=O)c1ccc(-n2cc(C#N)cn2)nc1-n1nc2cc(C(F)(F)F)ncc2c1Cl] | 1.50 | 482/484 | 3 | 251-253 |
| P11 | 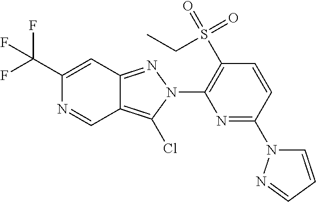 [SMILES: CCS(=O)(=O)c1ccc(-n2cccn2)nc1-n1nc2cc(C(F)(F)F)ncc2c1Cl] | 1.54 | 457/459 | 3 | 179-181 |
| P12 | 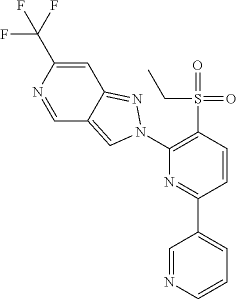 [SMILES: CCS(=O)(=O)c1ccc(-c2cccnc2)nc1-n1cc2cnc(C(F)(F)F)cc2n1] | 0.97 | 434 | 4 | 246-248 |
| P13 | 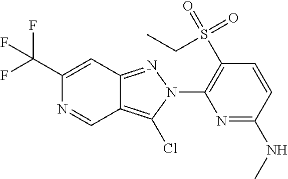 [SMILES: CCS(=O)(=O)c1ccc(NC)nc1-n1nc2cc(C(F)(F)F)ncc2c1Cl] | 0.97 | 420/422 | 4 | 257-259 |
| P14 | 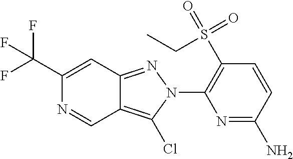 [SMILES: CCS(=O)(=O)c1ccc(N)nc1-n1nc2cc(C(F)(F)F)ncc2c1Cl] | 0.89 | 406/408 | 4 | 310-312 |
| P15 | 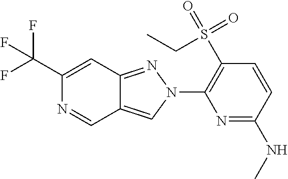 [SMILES: CCS(=O)(=O)c1ccc(NC)nc1-n1cc2cnc(C(F)(F)F)cc2n1] | 0.95 | 386 | 4 | 210-211 |
| P16 | 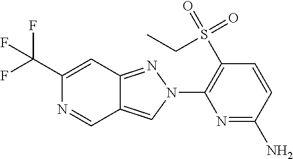 [SMILES: CCS(=O)(=O)c1ccc(N)nc1-n1cc2cnc(C(F)(F)F)cc2n1] | 0.86 | 372 | 4 | 224-226 |
| P17 | 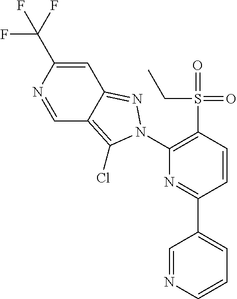 [SMILES: CCS(=O)(=O)c1ccc(-c2cccnc2)nc1-n1nc2cc(C(F)(F)F)ncc2c1Cl] | 1.01 | 468/470 | 4 | 194-196 |
| P18 | 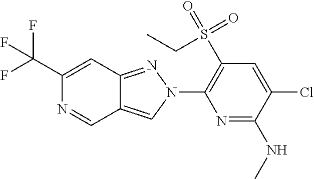 [SMILES: CCS(=O)(=O)c1cc(Cl)c(NC)nc1-n1cc2cnc(C(F)(F)F)cc2n1] | 1.00 | 420/422 | 4 | 242-244 |
| P19 | 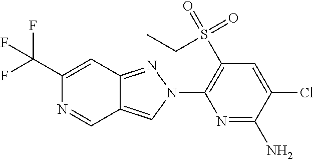 [SMILES: CCS(=O)(=O)c1cc(Cl)c(N)nc1-n1cc2cnc(C(F)(F)F)cc2n1] | 1.39 | 406/408 | 3 | 234-236 |
| P20 | 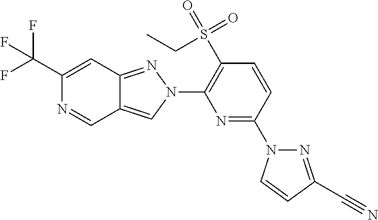 [SMILES: CCS(=O)(=O)c1ccc(-n2ccc(C#N)n2)nc1-n1cc2cnc(C(F)(F)F)cc2n1] | 1.53 | 448 | 3 | 288-290 |
| P21 | 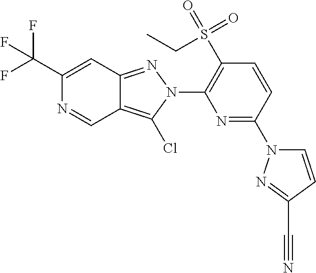 [SMILES: CCS(=O)(=O)c1ccc(-n2ccc(C#N)n2)nc1-n1nc2cc(C(F)(F)F)ncc2c1Cl] | 1.54 | 482/484 | 3 | 264-266 |
| P22 | 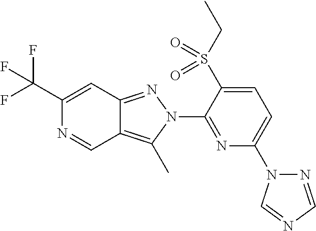 [SMILES: CCS(=O)(=O)c1ccc(-n2cncn2)nc1-n1nc2cc(C(F)(F)F)ncc2c1C] | 1.38 | 438 | 3 | 233-235 |
| P23 | 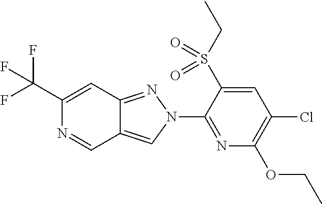 [SMILES: CCOc1nc(-n2cc3cnc(C(F)(F)F)cc3n2)c(S(=O)(=O)CC)cc1Cl] | 1.57 | 435/437 | 3 | 199-201 |
| P24 | 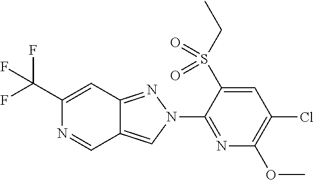 [SMILES: CCS(=O)(=O)c1cc(Cl)c(OC)nc1-n1cc2cnc(C(F)(F)F)cc2n1] | 1.51 | 421/423 | 3 | 226-228 |
| P25 | 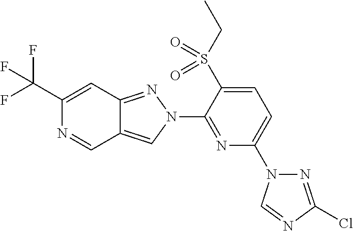 [SMILES: CCS(=O)(=O)c1ccc(-n2cnc(Cl)n2)nc1-n1cc2cnc(C(F)(F)F)cc2n1] | 1.48 | 458/460 | 3 | 243-245 |
| P26 | 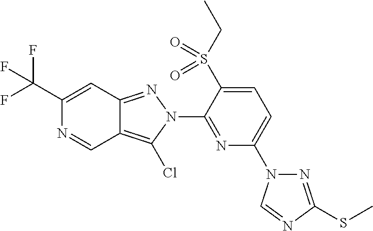 [SMILES: CCS(=O)(=O)c1ccc(-n2cnc(SC)n2)nc1-n1nc2cc(C(F)(F)F)ncc2c1Cl] | 1.53 | 504/506 | 3 | 118-120 |
| P27 | 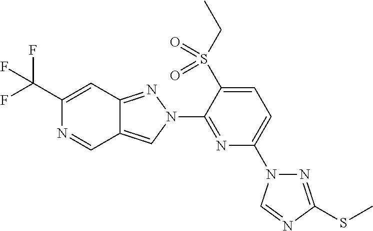 [SMILES: CCS(=O)(=O)c1ccc(-n2cnc(SC)n2)nc1-n1cc2cnc(C(F)(F)F)cc2n1] | 1.49 | 470 | 3 | 172-174 |
| P28 | 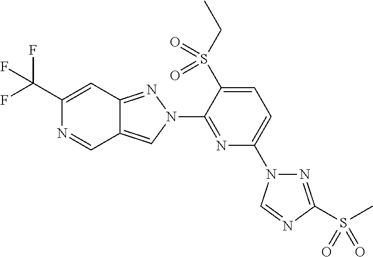 [SMILES: CCS(=O)(=O)c1ccc(-n2cnc(S(C)(=O)=O)n2)nc1-n1cc2cnc(C(F)(F)F)cc2n1] | 1.37 | 502 | 3 | 247-249 |
| P29 | 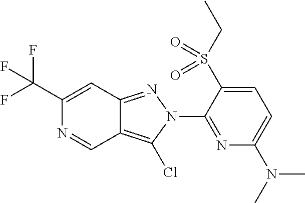 [SMILES: CCS(=O)(=O)c1ccc(N(C)C)nc1-n1nc2cc(C(F)(F)F)ncc2c1Cl] | 1.51 | 434/436 | 3 | 166-168 |
| P30 | 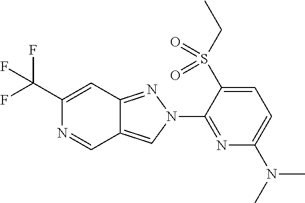 [SMILES: CCS(=O)(=O)c1ccc(N(C)C)nc1-n1cc2cnc(C(F)(F)F)cc2n1] | 1.45 | 400 | 3 | 256-258 |
| P31 | 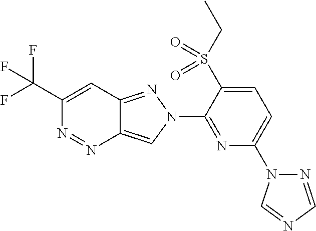 [SMILES: CCS(=O)(=O)c1ccc(-n2cncn2)nc1-n1cc2nnc(C(F)(F)F)cc2n1] | | | | |
| P32 | 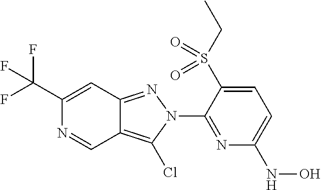 [SMILES: CCS(=O)(=O)c1ccc(NO)nc1-n1nc2cc(C(F)(F)F)ncc2c1Cl] | 0.91 | 422/424 | 4 | 246-248 |
| P33 | 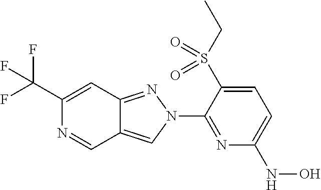 [SMILES: CCS(=O)(=O)c1ccc(NO)nc1-n1cc2cnc(C(F)(F)F)cc2n1] | 0.86 | 388 | 4 | |

The activity of the compositions according to the invention can be broadened considerably, and adapted to prevailing circumstances, by adding other insecticidally, acaricidally and/or fungicidally active ingredients. The mixtures of the compounds of formula I with other insecticidally, acaricidally and/or fungicidally active ingredients may also have further surprising advantages which can also be described, in a wider sense, as synergistic activity. For example, better tolerance by plants, reduced phytotoxicity, insects can be controlled in their different development stages or better behaviour during their production, for example during grinding or mixing, during their storage or during their use. Suitable additions to active ingredients here are, for example, representatives of the following classes of active ingredients: organophosphorus compounds, nitrophenol derivatives, thioureas, juvenile hormones, formamidines, benzophenone derivatives, ureas, pyrrole derivatives, carbamates, pyrethroids, chlorinated hydrocarbons, acylureas, pyridylmethyleneamino derivatives, macrolides, neonicotinoids and Bacillus thuringiensis preparations.

The following mixtures of the compounds of formula I with active ingredients are preferred (the abbreviation “TX” means “one compound selected from the group consisting of the compounds described in Tables 1, 2 and P of the present invention”): an adjuvant selected from the group of substances consisting of petroleum oils (628)+TX, an acaricide selected from the group of substances consisting of 1,1-bis(4-chlorophenyl)-2-ethoxyethanol (IUPAC name) (910)+TX, 2,4-dichlorophenyl benzenesulfonate (IUPAC/Chemical Abstracts name) (1059)+TX, 2-fluoro-N-methyl-N-1-naphthylacetamide (IUPAC name) (1295)+TX, 4-chlorophenyl phenyl sulfone (IUPAC name) (981)+TX, abamectin (1)+TX, acequinocyl (3)+TX, acetoprole [CCN]+TX, acrinathrin (9)+TX, aldicarb (16)+TX, aldoxycarb (863)+TX, alpha-cypermethrin (202)+TX, amidithion (870)+TX, amidoflumet [CCN]+TX, amidothioate (872)+TX, amiton (875)+TX, amiton hydrogen oxalate (875)+TX, amitraz (24)+TX, aramite (881)+TX, arsenous oxide (882)+TX, AVI 382 (compound code)+TX, AZ 60541 (compound code)+TX, azinphos-ethyl (44)+TX, azinphos-methyl (45)+TX, azobenzene (IUPAC name) (888)+TX, azocyclotin (46)+TX, azothoate (889)+TX, benomyl (62)+TX, benoxafos [CCN]+TX, benzoximate (71)+TX, benzyl benzoate (IUPAC name) [CCN]+TX, bifenazate (74)+TX, bifenthrin (76)+TX, binapacryl (907)+TX, brofenvalerate+TX, bromocyclen (918)+TX, bromophos (920)+TX, bromophos-ethyl (921)+TX, bromopropylate (94)+TX, buprofezin (99)+TX, butocarboxim (103)+TX, butoxycarboxim (104)+TX, butylpyridaben+TX, calcium polysulfide (IUPAC name) (111)+TX, camphechlor (941)+TX, carbanolate (943)+TX, carbaryl (115)+TX, carbofuran (118)+TX, carbophenothion (947)+TX, CGA 50′439 (development code) (125)+TX, chinomethionat (126)+TX, chlorbenside (959)+TX, chlordimeform (964)+TX, chlordimeform hydrochloride (964)+TX, chlorfenapyr (130)+TX, chlorfenethol (968)+TX, chlorfenson (970)+TX, chlorfensulfide (971)+TX, chlorfenvinphos (131)+TX, chlorobenzilate (975)+TX, chloromebuform (977)+TX, chloromethiuron (978)+TX, chloropropylate (983)+TX, chlorpyrifos (145)+TX, chlorpyrifos-methyl (146)+TX, chlorthiophos (994)+TX, cinerin I (696)+TX, cinerin II (696)+TX, cinerins (696)+TX, clofentezine (158)+TX, closantel [CCN]+TX, coumaphos (174)+TX, crotamiton [CCN]+TX, crotoxyphos (1010)+TX, cufraneb (1013)+TX, cyanthoate (1020)+TX, cyflumetofen (CAS Reg. No.: 400882-07-7)+TX, cyhalothrin (196)+TX, cyhexatin (199)+TX, cypermethrin (201)+TX, DCPM (1032)+TX, DDT (219)+TX, demephion (1037)+TX, demephion-O (1037)+TX, demephion-S(1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulfon (1039)+TX, diafenthiuron (226)+TX, dialifos (1042)+TX, diazinon (227)+TX, dichlofluanid (230)+TX, dichlorvos (236)+TX, dicliphos+TX, dicofol (242)+TX, dicrotophos (243)+TX, dienochlor (1071)+TX, dimefox (1081)+TX, dimethoate (262)+TX, dinactin (653)+TX, dinex (1089)+TX, dinex-diclexine (1089)+TX, dinobuton (269)+TX, dinocap (270)+TX, dinocap-4 [CCN]+TX, dinocap-6 [CCN]+TX, dinocton (1090)+TX, dinopenton (1092)+TX, dinosulfon (1097)+TX, dinoterbon (1098)+TX, dioxathion (1102)+TX, diphenyl sulfone (IUPAC name) (1103)+TX, disulfiram [CCN]+TX, disulfoton (278)+TX, DNOC (282)+TX, dofenapyn (1113)+TX, doramectin [CCN]+TX, endosulfan (294)+TX, endothion (1121)+TX, EPN (297)+TX, eprinomectin [CCN]+TX, ethion (309)+TX, ethoate-methyl (1134)+TX, etoxazole (320)+TX, etrimfos (1142)+TX, fenazaflor (1147)+TX, fenazaquin (328)+TX, fenbutatin oxide (330)+TX, fenothiocarb (337)+TX, fenpropathrin (342)+TX, fenpyrad+TX, fenpyroximate (345)+TX, fenson (1157)+TX, fentrifanil (1161)+TX, fenvalerate (349)+TX, fipronil (354)+TX, fluacrypyrim (360)+TX, fluazuron (1166)+TX, flubenzimine (1167)+TX, flucycloxuron (366)+TX, flucythrinate (367)+TX, fluenetil (1169)+TX, flufenoxuron (370)+TX, flumethrin (372)+TX, fluorbenside (1174)+TX, fluvalinate (1184)+TX, FMC 1137 (development code) (1185)+TX, formetanate (405)+TX, formetanate hydrochloride (405)+TX, formothion (1192)+TX, formparanate (1193)+TX, gamma-HCH (430)+TX, glyodin (1205)+TX, halfenprox (424)+TX, heptenophos (432)+TX, hexadecyl cyclopropanecarboxylate (IUPAC/Chemical Abstracts name) (1216)+TX, hexythiazox (441)+TX, iodomethane (IUPAC name) (542)+TX, isocarbophos (473)+TX, isopropyl 0-(methoxyaminothiophosphoryl)salicylate (IUPAC name) (473)+TX, ivermectin [CCN]+TX, jasmolin I (696)+TX, jasmolin II (696)+TX, jodfenphos (1248)+TX, lindane (430)+TX, lufenuron (490)+TX, malathion (492)+TX, malonoben (1254)+TX, mecarbam (502)+TX, mephosfolan (1261)+TX, mesulfen [CCN]+TX, methacrifos (1266)+TX, methamidophos (527)+TX, methidathion (529)+TX, methiocarb (530)+TX, methomyl (531)+TX, methyl bromide (537)+TX, metolcarb (550)+TX, mevinphos (556)+TX, mexacarbate (1290)+TX, milbemectin (557)+TX, milbemycin oxime [CCN]+TX, mipafox (1293)+TX, monocrotophos (561)+TX, morphothion (1300)+TX, moxidectin [CCN]+TX, naled (567)+TX, NC-184 (compound code)+TX, NC-512 (compound code)+TX, nifluridide (1309)+TX, nikkomycins [CCN]+TX, nitrilacarb (1313)+TX, nitrilacarb 1:1 zinc chloride complex (1313)+TX, NNI-0101 (compound code)+TX, NNI-0250 (compound code)+TX, omethoate (594)+TX, oxamyl (602)+TX, oxydeprofos (1324)+TX, oxydisulfoton (1325)+TX, pp′-DDT (219)+TX, parathion (615)+TX, permethrin (626)+TX, petroleum oils (628)+TX, phenkapton (1330)+TX, phenthoate (631)+TX, phorate (636)+TX, phosalone (637)+TX, phosfolan (1338)+TX, phosmet (638)+TX, phosphamidon (639)+TX, phoxim (642)+TX, pirimiphos-methyl (652)+TX, polychloroterpenes (traditional name) (1347)+TX, polynactins (653)+TX, proclonol (1350)+TX, profenofos (662)+TX, promacyl (1354)+TX, propargite (671)+TX, propetamphos (673)+TX, propoxur (678)+TX, prothidathion (1360)+TX, prothoate (1362)+TX, pyrethrin I (696)+TX, pyrethrin II (696)+TX, pyrethrins (696)+TX, pyridaben (699)+TX, pyridaphenthion (701)+TX, pyrimidifen (706)+TX, pyrimitate (1370)+TX, quinalphos (711)+TX, quintiofos (1381)+TX, R-1492 (development code) (1382)+TX, RA-17 (development code) (1383)+TX, rotenone (722)+TX, schradan (1389)+TX, sebufos+TX, selamectin [CCN]+TX, SI-0009 (compound code)+TX, sophamide (1402)+TX, spirodiclofen (738)+TX, spiromesifen (739)+TX, SSI-121 (development code) (1404)+TX, sulfiram [CCN]+TX, sulfluramid (750)+TX, sulfotep (753)+TX, sulfur (754)+TX, SZI-121 (development code) (757)+TX, tau-fluvalinate (398)+TX, tebufenpyrad (763)+TX, TEPP (1417)+TX, terbam+TX, tetrachlorvinphos (777)+TX, tetradifon (786)+TX, tetranactin (653)+TX, tetrasul (1425)+TX, thiafenox+TX, thiocarboxime (1431)+TX, thiofanox (800)+TX, thiometon (801)+TX, thioquinox (1436)+TX, thuringiensin [CCN]+TX, triamiphos (1441)+TX, triarathene (1443)+TX, triazophos (820)+TX, triazuron+TX, trichlorfon (824)+TX, trifenofos (1455)+TX, trinactin (653)+TX, vamidothion (847)+TX, vaniliprole [CCN] and YI-5302 (compound code)+TX,

an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX,

an anthelmintic selected from the group of substances consisting of abamectin (1)+TX, crufomate (1011)+TX, doramectin [CCN]+TX, emamectin (291)+TX, emamectin benzoate (291)+TX, eprinomectin [CCN]+TX, ivermectin [CCN]+TX, milbemycin oxime [CCN]+TX, moxidectin [CCN]+TX, piperazine [CCN]+TX, selamectin [CCN]+TX, spinosad (737) and thiophanate (1435)+TX,

an avicide selected from the group of substances consisting of chloralose (127)+TX, endrin (1122)+TX, fenthion (346)+TX, pyridin-4-amine (IUPAC name) (23) and strychnine (745)+TX, a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal [CCN]+TX, a biological agent selected from the group of substances consisting of Adoxophyes orana GV (12)+TX, Agrobacterium radiobacter (13)+TX, Amblyseius spp. (19)+TX, Anagrapha falcifera NPV (28)+TX, Anagrus atomus (29)+TX, Aphelinus abdominalis (33)+TX, Aphidius colemani (34)+TX, Aphidoletes aphidimyza (35)+TX, Autographa californica NPV (38)+TX, Bacillus firmus (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (53)+TX, Beauveria brongniartii (54)+TX, Chrysoperla carnea (151)+TX, Cryptolaemus montrouzieri (178)+TX, Cydia pomonella GV (191)+TX, Dacnusa sibirica (212)+TX, Diglyphus isaea (254)+TX, Encarsia formosa (scientific name) (293)+TX, Eretmocerus eremicus (300)+TX, Helicoverpa zea NPV (431)+TX, Heterorhabditis bacteriophora and H. megidis (433)+TX, Hippodamia convergens (442)+TX, Leptomastix dactylopii (488)+TX, Macrolophus caliginosus (491)+TX, Mamestra brassicae NPV (494)+TX, Metaphycus helvolus (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (575)+TX, Orius spp. (596)+TX, Paecilomyces fumosoroseus (613)+TX, Phytoseiulus persimilis (644)+TX, Spodoptera exigua multicapsid nuclear polyhedrosis virus (scientific name) (741)+TX, Steinernema bibionis (742)+TX, Steinernema carpocapsae (742)+TX, Steinernema feltiae (742)+TX, Steinernema glaseri (742)+TX, Steinernema riobrave (742)+TX, Steinernema riobravis (742)+TX, Steinernema scapterisci (742)+TX, Steinernema spp. (742)+TX, Trichogramma spp. (826)+TX, Typhlodromus occidentalis (844) and Verticillium lecanii (848)+TX,

a soil sterilant selected from the group of substances consisting of iodomethane (IUPAC name) (542) and methyl bromide (537)+TX,

a chemosterilant selected from the group of substances consisting of apholate [CCN]+TX, bisazir [CCN]+TX, busulfan [CCN]+TX, diflubenzuron (250)+TX, dimatif [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron [CCN]+TX, tepa [CCN]+TX, thiohempa [CCN]+TX, thiotepa [CCN]+TX, tretamine [CCN] and uredepa [CCN]+TX,

an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin [CCN]+TX, brevicomin [CCN]+TX, codlelure [CCN]+TX, codlemone (167)+TX, cuelure (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure [CCN]+TX, ethyl 4-methyloctanoate (IUPAC name) (317)+TX, eugenol [CCN]+TX, frontalin [CCN]+TX, gossyplure (420)+TX, grandlure (421)+TX, grandlure I (421)+TX, grandlure II (421)+TX, grandlure III (421)+TX, grandlure IV (421)+TX, hexalure [CCN]+TX, ipsdienol [CCN]+TX, ipsenol [CCN]+TX, japonilure (481)+TX, lineatin [CCN]+TX, litlure [CCN]+TX, looplure [CCN]+TX, medlure [CCN]+TX, megatomoic acid [CCN]+TX, methyl eugenol (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure [CCN]+TX, oryctalure (317)+TX, ostramone [CCN]+TX, siglure [CCN]+TX, sordidin (736)+TX, sulcatol [CCN]+TX, tetradec-11-en-1-yl acetate (IUPAC name) (785)+TX, trimedlure (839)+TX, trimedlure A (839)+TX, trimedlure B₁ (839)+TX, trimedlure B2 (839)+TX, trimedlure C (839) and trunc-call [CCN]+TX,

an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX, an insecticide selected from the group of substances consisting of 1-dichloro-1-nitroethane (IUPAC/Chemical Abstracts name) (1058)+TX, 1,1-dichloro-2,2-bis(4-ethylphenyl)ethane (IUPAC name) (1056), +TX, 1,2-dichloropropane (IUPAC/Chemical Abstracts name) (1062)+TX, 1,2-dichloropropane with 1,3-dichloropropene (IUPAC name) (1063)+TX, 1-bromo-2-chloroethane (IUPAC/Chemical Abstracts name) (916)+TX, 2,2,2-trichloro-1-(3,4-dichlorophenyl)ethyl acetate (IUPAC name) (1451)+TX, 2,2-dichlorovinyl 2-ethylsulfinylethyl methyl phosphate (IUPAC name) (1066)+TX, 2-(1,3-dithiolan-2-yl)phenyl dimethylcarbamate (IUPAC/Chemical Abstracts name) (1109)+TX, 2-(2-butoxyethoxy)ethyl thiocyanate (IUPAC/Chemical Abstracts name) (935)+TX, 2-(4,5-dimethyl-1,3-dioxolan-2-yl)phenyl methylcarbamate (IUPAC/Chemical Abstracts name) (1084)+TX, 2-(4-chloro-3,5-xylyloxy)ethanol (IUPAC name) (986)+TX, 2-chlorovinyl diethyl phosphate (IUPAC name) (984)+TX, 2-imidazolidone (IUPAC name) (1225)+TX, 2-isovalerylindan-1,3-dione (IUPAC name) (1246)+TX, 2-methyl(prop-2-ynyl)aminophenyl methylcarbamate (IUPAC name) (1284)+TX, 2-thiocyanatoethyl laurate (IUPAC name) (1433)+TX, 3-bromo-1-chloroprop-1-ene (IUPAC name) (917)+TX, 3-methyl-1-phenylpyrazol-5-yl dimethylcarbamate (IUPAC name) (1283)+TX, 4-methyl(prop-2-ynyl)amino-3,5-xylyl methylcarbamate (IUPAC name) (1285)+TX, 5,5-dimethyl-3-oxocyclohex-1-enyl dimethylcarbamate (IUPAC name) (1085)+TX, abamectin (1)+TX, acephate (2)+TX, acetamiprid (4)+TX, acethion [CCN]+TX, acetoprole [CCN]+TX, acrinathrin (9)+TX, acrylonitrile (IUPAC name) (861)+TX, alanycarb (15)+TX, aldicarb (16)+TX, aldoxycarb (863)+TX, aldrin (864)+TX, allethrin (17)+TX, allosamidin [CCN]+TX, allyxycarb (866)+TX, alpha-cypermethrin (202)+TX, alpha-ecdysone [CCN]+TX, aluminium phosphide (640)+TX, amidithion (870)+TX, amidothioate (872)+TX, aminocarb (873)+TX, amiton (875)+TX, amiton hydrogen oxalate (875)+TX, amitraz (24)+TX, anabasine (877)+TX, athidathion (883)+TX, AVI 382 (compound code)+TX, AZ 60541 (compound code)+TX, azadirachtin (41)+TX, azamethiphos (42)+TX, azinphos-ethyl (44)+TX, azinphos-methyl (45)+TX, azothoate (889)+TX, Bacillus thuringiensis delta endotoxins (52)+TX, barium hexafluorosilicate [CCN]+TX, barium polysulfide (IUPAC/Chemical Abstracts name) (892)+TX, barthrin [CCN]+TX, Bayer 22/190 (development code) (893)+TX, Bayer 22408 (development code) (894)+TX, bendiocarb (58)+TX, benfuracarb (60)+TX, bensultap (66)+TX, beta-cyfluthrin (194)+TX, beta-cypermethrin (203)+TX, bifenthrin (76)+TX, bioallethrin (78)+TX, bioallethrin S-cyclopentenyl isomer (79)+TX, bioethanomethrin [CCN]+TX, biopermethrin (908)+TX, bioresmethrin (80)+TX, bis(2-chloroethyl) ether (IUPAC name) (909)+TX, bistrifluron (83)+TX, borax (86)+TX, brofenvalerate+TX, bromfenvinfos (914)+TX, bromocyclen (918)+TX, bromo-DDT [CCN]+TX, bromophos (920)+TX, bromophos-ethyl (921)+TX, bufencarb (924)+TX, buprofezin (99)+TX, butacarb (926)+TX, butathiofos (927)+TX, butocarboxim (103)+TX, butonate (932)+TX, butoxycarboxim (104)+TX, butylpyridaben+TX, cadusafos (109)+TX, calcium arsenate [CCN]+TX, calcium cyanide (444)+TX, calcium polysulfide (IUPAC name) (111)+TX, camphechlor (941)+TX, carbanolate (943)+TX, carbaryl (115)+TX, carbofuran (118)+TX, carbon disulfide (IUPAC/Chemical Abstracts name) (945)+TX, carbon tetrachloride (IUPAC name) (946)+TX, carbophenothion (947)+TX, carbosulfan (119)+TX, cartap (123)+TX, cartap hydrochloride (123)+TX, cevadine (725)+TX, chlorbicyclen (960)+TX, chlordane (128)+TX, chlordecone (963)+TX, chlordimeform (964)+TX, chlordimeform hydrochloride (964)+TX, chlorethoxyfos (129)+TX, chlorfenapyr (130)+TX, chlorfenvinphos (131)+TX, chlorfluazuron (132)+TX, chlormephos (136)+TX, chloroform [CCN]+TX, chloropicrin (141)+TX, chlorphoxim (989)+TX, chlorprazophos (990)+TX, chlorpyrifos (145)+TX, chlorpyrifos-methyl (146)+TX, chlorthiophos (994)+TX, chromafenozide (150)+TX, cinerin I (696)+TX, cinerin II (696)+TX, cinerins (696)+TX, cis-resmethrin+TX, cismethrin (80)+TX, clocythrin+TX, cloethocarb (999)+TX, closantel [CCN]+TX, clothianidin (165)+TX, copper acetoarsenite [CCN]+TX, copper arsenate [CCN]+TX, copper oleate [CCN]+TX, coumaphos (174)+TX, coumithoate (1006)+TX, crotamiton [CCN]+TX, crotoxyphos (1010)+TX, crufomate (1011)+TX, cryolite (177)+TX, CS 708 (development code) (1012)+TX, cyanofenphos (1019)+TX, cyanophos (184)+TX, cyanthoate (1020)+TX, cyclethrin [CCN]+TX, cycloprothrin (188)+TX, cyfluthrin (193)+TX, cyhalothrin (196)+TX, cypermethrin (201)+TX, cyphenothrin (206)+TX, cyromazine (209)+TX, cythioate [CCN]+TX, d-limonene [CCN]+TX, d-tetramethrin (788)+TX, DAEP (1031)+TX, dazomet (216)+TX, DDT (219)+TX, decarbofuran (1034)+TX, deltamethrin (223)+TX, demephion (1037)+TX, demephion-O (1037)+TX, demephion-S(1037)+TX, demeton (1038)+TX, demeton-methyl (224)+TX, demeton-O (1038)+TX, demeton-O-methyl (224)+TX, demeton-S(1038)+TX, demeton-S-methyl (224)+TX, demeton-S-methylsulphon (1039)+TX, diafenthiuron (226)+TX, dialifos (1042)+TX, diamidafos (1044)+TX, diazinon (227)+TX, dicapthon (1050)+TX, dichlofenthion (1051)+TX, dichlorvos (236)+TX, dicliphos+TX, dicresyl [CCN]+TX, dicrotophos (243)+TX, dicyclanil (244)+TX, dieldrin (1070)+TX, diethyl 5-methylpyrazol-3-yl phosphate (IUPAC name) (1076)+TX, diflubenzuron (250)+TX, dilor [CCN]+TX, dimefluthrin [CCN]+TX, dimefox (1081)+TX, dimetan (1085)+TX, dimethoate (262)+TX, dimethrin (1083)+TX, dimethylvinphos (265)+TX, dimetilan (1086)+TX, dinex (1089)+TX, dinex-diclexine (1089)+TX, dinoprop (1093)+TX, dinosam (1094)+TX, dinoseb (1095)+TX, dinotefuran (271)+TX, diofenolan (1099)+TX, dioxabenzofos (1100)+TX, dioxacarb (1101)+TX, dioxathion (1102)+TX, disulfoton (278)+TX, dithicrofos (1108)+TX, DNOC (282)+TX, doramectin [CCN]+TX, DSP (1115)+TX, ecdysterone [CCN]+TX, EI 1642 (development code) (1118)+TX, emamectin (291)+TX, emamectin benzoate (291)+TX, EMPC (1120)+TX, empenthrin (292)+TX, endosulfan (294)+TX, endothion (1121)+TX, endrin (1122)+TX, EPBP (1123)+TX, EPN (297)+TX, epofenonane (1124)+TX, eprinomectin [CCN]+TX, esfenvalerate (302)+TX, etaphos [CCN]+TX, ethiofencarb (308)+TX, ethion (309)+TX, ethiprole (310)+TX, ethoate-methyl (1134)+TX, ethoprophos (312)+TX, ethyl formate (IUPAC name) [CCN]+TX, ethyl-DDD (1056)+TX, ethylene dibromide (316)+TX, ethylene dichloride (chemical name) (1136)+TX, ethylene oxide [CCN]+TX, etofenprox (319)+TX, etrimfos (1142)+TX, EXD (1143)+TX, famphur (323)+TX, fenamiphos (326)+TX, fenazaflor (1147)+TX, fenchlorphos (1148)+TX, fenethacarb (1149)+TX, fenfluthrin (1150)+TX, fenitrothion (335)+TX, fenobucarb (336)+TX, fenoxacrim (1153)+TX, fenoxycarb (340)+TX, fenpirithrin (1155)+TX, fenpropathrin (342)+TX, fenpyrad+TX, fensulfothion (1158)+TX, fenthion (346)+TX, fenthion-ethyl [CCN]+TX, fenvalerate (349)+TX, fipronil (354)+TX, flonicamid (358)+TX, flubendiamide (CAS. Reg. No.: 272451-65-7)+TX, flucofuron (1168)+TX, flucycloxuron (366)+TX, flucythrinate (367)+TX, fluenetil (1169)+TX, flufenerim [CCN]+TX, flufenoxuron (370)+TX, flufenprox (1171)+TX, flumethrin (372)+TX, fluvalinate (1184)+TX, FMC 1137 (development code) (1185)+TX, fonofos (1191)+TX, formetanate (405)+TX, formetanate hydrochloride (405)+TX, formothion (1192)+TX, formparanate (1193)+TX, fosmethilan (1194)+TX, fospirate (1195)+TX, fosthiazate (408)+TX, fosthietan (1196)+TX, furathiocarb (412)+TX, furethrin (1200)+TX, gamma-cyhalothrin (197)+TX, gamma-HCH (430)+TX, guazatine (422)+TX, guazatine acetates (422)+TX, GY-81 (development code) (423)+TX, halfenprox (424)+TX, halofenozide (425)+TX, HCH (430)+TX, HEOD (1070)+TX, heptachlor (1211)+TX, heptenophos (432)+TX, heterophos [CCN]+TX, hexaflumuron (439)+TX, HHDN (864)+TX, hydramethylnon (443)+TX, hydrogen cyanide (444)+TX, hydroprene (445)+TX, hyquincarb (1223)+TX, imidacloprid (458)+TX, imiprothrin (460)+TX, indoxacarb (465)+TX, iodomethane (IUPAC name) (542)+TX, IPSP (1229)+TX, isazofos (1231)+TX, isobenzan (1232)+TX, isocarbophos (473)+TX, isodrin (1235)+TX, isofenphos (1236)+TX, isolane (1237)+TX, isoprocarb (472)+TX, isopropyl O-(methoxyaminothiophosphoryl)salicylate (IUPAC name) (473)+TX, isoprothiolane (474)+TX, isothioate (1244)+TX, isoxathion (480)+TX, ivermectin [CCN]+TX, jasmolin I (696)+TX, jasmolin II (696)+TX, jodfenphos (1248)+TX, juvenile hormone I [CCN]+TX, juvenile hormone II [CCN]+TX, juvenile hormone III [CCN]+TX, kelevan (1249)+TX, kinoprene (484)+TX, lambda-cyhalothrin (198)+TX, lead arsenate [CCN]+TX, lepimectin (CCN)+TX, leptophos (1250)+TX, lindane (430)+TX, lirimfos (1251)+TX, lufenuron (490)+TX, lythidathion (1253)+TX, m-cumenyl methylcarbamate (IUPAC name) (1014)+TX, magnesium phosphide (IUPAC name) (640)+TX, malathion (492)+TX, malonoben (1254)+TX, mazidox (1255)+TX, mecarbam (502)+TX, mecarphon (1258)+TX, menazon (1260)+TX, mephosfolan (1261)+TX, mercurous chloride (513)+TX, mesulfenfos (1263)+TX, metaflumizone (CCN)+TX, metam (519)+TX, metam-potassium (519)+TX, metam-sodium (519)+TX, methacrifos (1266)+TX, methamidophos (527)+TX, methanesulfonyl fluoride (IUPAC/Chemical Abstracts name) (1268)+TX, methidathion (529)+TX, methiocarb (530)+TX, methocrotophos (1273)+TX, methomyl (531)+TX, methoprene (532)+TX, methoquin-butyl (1276)+TX, methothrin (533)+TX, methoxychlor (534)+TX, methoxyfenozide (535)+TX, methyl bromide (537)+TX, methyl isothiocyanate (543)+TX, methylchloroform [CCN]+TX, methylene chloride [CCN]+TX, metofluthrin [CCN]+TX, metolcarb (550)+TX, metoxadiazone (1288)+TX, mevinphos (556)+TX, mexacarbate (1290)+TX, milbemectin (557)+TX, milbemycin oxime [CCN]+TX, mipafox (1293)+TX, mirex (1294)+TX, monocrotophos (561)+TX, morphothion (1300)+TX, moxidectin [CCN]+TX, naftalofos [CCN]+TX, naled (567)+TX, naphthalene (IUPAC/Chemical Abstracts name) (1303)+TX, NC-170 (development code) (1306)+TX, NC-184 (compound code)+TX, nicotine (578)+TX, nicotine sulfate (578)+TX, nifluridide (1309)+TX, nitenpyram (579)+TX, nithiazine (1311)+TX, nitrilacarb (1313)+TX, nitrilacarb 1:1 zinc chloride complex (1313)+TX, NNI-0101 (compound code)+TX, NNI-0250 (compound code)+TX, nornicotine (traditional name) (1319)+TX, novaluron (585)+TX, noviflumuron (586)+TX, O-5-dichloro-4-iodophenyl O-ethyl ethylphosphonothioate (IUPAC name) (1057)+TX, O,O-diethyl O-4-methyl-2-oxo-2H-chromen-7-yl phosphorothioate (IUPAC name) (1074)+TX, O,O-diethyl O-6-methyl-2-propylpyrimidin-4-yl phosphorothioate (IUPAC name) (1075)+TX, O,O,O′,O′-tetrapropyl dithiopyrophosphate (IUPAC name) (1424)+TX, oleic acid (IUPAC name) (593)+TX, omethoate (594)+TX, oxamyl (602)+TX, oxydemeton-methyl (609)+TX, oxydeprofos (1324)+TX, oxydisulfoton (1325)+TX, pp′-DDT (219)+TX, para-dichlorobenzene [CCN]+TX, parathion (615)+TX, parathion-methyl (616)+TX, penfluron [CCN]+TX, pentachlorophenol (623)+TX, pentachlorophenyl laurate (IUPAC name) (623)+TX, permethrin (626)+TX, petroleum oils (628)+TX, PH 60-38 (development code) (1328)+TX, phenkapton (1330)+TX, phenothrin (630)+TX, phenthoate (631)+TX, phorate (636)+TX, phosalone (637)+TX, phosfolan (1338)+TX, phosmet (638)+TX, phosnichlor (1339)+TX, phosphamidon (639)+TX, phosphine (IUPAC name) (640)+TX, phoxim (642)+TX, phoxim-methyl (1340)+TX, pirimetaphos (1344)+TX, pirimicarb (651)+TX, pirimiphos-ethyl (1345)+TX, pirimiphos-methyl (652)+TX, polychlorodicyclopentadiene isomers (IUPAC name) (1346)+TX, polychloroterpenes (traditional name) (1347)+TX, potassium arsenite [CCN]+TX, potassium thiocyanate [CCN]+TX, prallethrin (655)+TX, precocene I [CCN]+TX, precocene II [CCN]+TX, precocene Ill [CCN]+TX, primidophos (1349)+TX, profenofos (662)+TX, profluthrin [CCN]+TX, promacyl (1354)+TX, promecarb (1355)+TX, propaphos (1356)+TX, propetamphos (673)+TX, propoxur (678)+TX, prothidathion (1360)+TX, prothiofos (686)+TX, prothoate (1362)+TX, protrifenbute [CCN]+TX, pymetrozine (688)+TX, pyraclofos (689)+TX, pyrazophos (693)+TX, pyresmethrin (1367)+TX, pyrethrin I (696)+TX, pyrethrin II (696)+TX, pyrethrins (696)+TX, pyridaben (699)+TX, pyridalyl (700)+TX, pyridaphenthion (701)+TX, pyrimidifen (706)+TX, pyrimitate (1370)+TX, pyriproxyfen (708)+TX, quassia [CCN]+TX, quinalphos (711)+TX, quinalphos-methyl (1376)+TX, quinothion (1380)+TX, quintiofos (1381)+TX, R-1492 (development code) (1382)+TX, rafoxanide [CCN]+TX, resmethrin (719)+TX, rotenone (722)+TX, RU 15525 (development code) (723)+TX, RU 25475 (development code) (1386)+TX, ryania (1387)+TX, ryanodine (traditional name) (1387)+TX, sabadilla (725)+TX, schradan (1389)+TX, sebufos+TX, selamectin [CCN]+TX, SI-0009 (compound code)+TX, SI-0205 (compound code)+TX, SI-0404 (compound code)+TX, SI-0405 (compound code)+TX, silafluofen (728)+TX, SN 72129 (development code) (1397)+TX, sodium arsenite [CCN]+TX, sodium cyanide (444)+TX, sodium fluoride (IUPAC/Chemical Abstracts name) (1399)+TX, sodium hexafluorosilicate (1400)+TX, sodium pentachlorophenoxide (623)+TX, sodium selenate (IUPAC name) (1401)+TX, sodium thiocyanate [CCN]+TX, sophamide (1402)+TX, spinosad (737)+TX, spiromesifen (739)+TX, spirotetrmat (CCN)+TX, sulcofuron (746)+TX, sulcofuron-sodium (746)+TX, sulfluramid (750)+TX, sulfotep (753)+TX, sulfuryl fluoride (756)+TX, sulprofos (1408)+TX, tar oils (758)+TX, tau-fluvalinate (398)+TX, tazimcarb (1412)+TX, TDE (1414)+TX, tebufenozide (762)+TX, tebufenpyrad (763)+TX, tebupirimfos (764)+TX, teflubenzuron (768)+TX, tefluthrin (769)+TX, temephos (770)+TX, TEPP (1417)+TX, terallethrin (1418)+TX, terbam+TX, terbufos (773)+TX, tetrachloroethane [CCN]+TX, tetrachlorvinphos (777)+TX, tetramethrin (787)+TX, theta-cypermethrin (204)+TX, thiacloprid (791)+TX, thiafenox+TX, thiamethoxam (792)+TX, thicrofos (1428)+TX, thiocarboxime (1431)+TX, thiocyclam (798)+TX, thiocyclam hydrogen oxalate (798)+TX, thiodicarb (799)+TX, thiofanox (800)+TX, thiometon (801)+TX, thionazin (1434)+TX, thiosultap (803)+TX, thiosultap-sodium (803)+TX, thuringiensin [CCN]+TX, tolfenpyrad (809)+TX, tralomethrin (812)+TX, transfluthrin (813)+TX, transpermethrin (1440)+TX, triamiphos (1441)+TX, triazamate (818)+TX, triazophos (820)+TX, triazuron+TX, trichlorfon (824)+TX, trichlormetaphos-3 [CCN]+TX, trichloronat (1452)+TX, trifenofos (1455)+TX, triflumuron (835)+TX, trimethacarb (840)+TX, triprene (1459)+TX, vamidothion (847)+TX, vaniliprole [CCN]+TX, veratridine (725)+TX, veratrine (725)+TX, XMC (853)+TX, xylylcarb (854)+TX, YI-5302 (compound code)+TX, zeta-cypermethrin (205)+TX, zetamethrin+TX, zinc phosphide (640)+TX, zolaprofos (1469) and ZXI 8901 (development code) (858)+TX, cyantraniliprole [736994-63-19+TX, chlorantraniliprole [500008-45-7]+TX, cyenopyrafen [560121-52-0]+TX, cyflumetofen [400882-07-7]+TX, pyrifluquinazon [337458-27-2]+TX, spinetoram [187166-40-1+187166-15-0]+TX, spirotetramat [203313-25-1]+TX, sulfoxaflor [946578-00-3]+TX, flufiprole [704886-18-0]+TX, meperfluthrin [915288-13-0]+TX, tetramethylfluthrin [84937-88-2]+TX, triflumezopyrim (disclosed in WO 2012/092115)+TX,

a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX,

a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cytokinins (210)+TX, dazomet (216)+TX,

DBCP (1045)+TX, DCIP (218)+TX, diamidafos (1044)+TX, dichlofenthion (1051)+TX, dicliphos+TX, dimethoate (262)+TX, doramectin [CCN]+TX, emamectin (291)+TX, emamectin benzoate (291)+TX, eprinomectin [CCN]+TX, ethoprophos (312)+TX, ethylene dibromide (316)+TX, fenamiphos (326)+TX, fenpyrad+TX, fensulfothion (1158)+TX, fosthiazate (408)+TX, fosthietan (1196)+TX, furfural [CCN]+TX, GY-81 (development code) (423)+TX, heterophos [CCN]+TX, iodomethane (IUPAC name) (542)+TX, isamidofos (1230)+TX, isazofos (1231)+TX, ivermectin [CCN]+TX, kinetin (210)+TX, mecarphon (1258)+TX, metam (519)+TX, metam-potassium (519)+TX, metam-sodium (519)+TX, methyl bromide (537)+TX, methyl isothiocyanate (543)+TX, milbemycin oxime [CCN]+TX, moxidectin [CCN]+TX, Myrothecium verrucaria composition (565)+TX, NC-184 (compound code)+TX, oxamyl (602)+TX, phorate (636)+TX, phosphamidon (639)+TX, phosphocarb [CCN]+TX, sebufos+TX, selamectin [CCN]+TX, spinosad (737)+TX, terbam+TX, terbufos (773)+TX, tetrachlorothiophene (IUPAC/Chemical Abstracts name) (1422)+TX, thiafenox+TX, thionazin (1434)+TX, triazophos (820)+TX, triazuron+TX, xylenols [CCN]+TX, YI-5302 (compound code) and zeatin (210)+TX, fluensulfone [318290-98-1]+TX,

a nitrification inhibitor selected from the group of substances consisting of potassium ethylxanthate [CCN] and nitrapyrin (580)+TX,

a plant activator selected from the group of substances consisting of acibenzolar (6)+TX, acibenzolar-S-methyl (6)+TX, probenazole (658) and Reynoutria sachalinensis extract (720)+TX, a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (91)+TX, bromethalin (92)+TX, calcium cyanide (444)+TX, chloralose (127)+TX, chlorophacinone (140)+TX, cholecalciferol (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX,

a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX,

an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX,

a virucide selected from the group of substances consisting of imanin [CCN] and ribavirin [CCN]+TX,

a wound protectant selected from the group of substances consisting of mercuric oxide (512)+TX, octhilinone (590) and thiophanate-methyl (802)+TX,

and biologically active compounds selected from the group consisting of azaconazole (60207-31-0]+TX, bitertanol [70585-36-3]+TX, bromuconazole [116255-48-2]+TX, cyproconazole [94361-06-5]+TX, difenoconazole [119446-68-3]+TX, diniconazole [83657-24-3]+TX, epoxiconazole [106325-08-0]+TX, fenbuconazole [114369-43-6]+TX, fluquinconazole [136426-54-5]+TX, flusilazole [85509-19-9]+TX, flutriafol [76674-21-0]+TX, hexaconazole [79983-71-4]+TX, imazalil [35554-44-0]+TX, imibenconazole [86598-92-7]+TX, ipconazole [125225-28-7]+TX, metconazole [125116-23-6]+TX, myclobutanil [88671-89-0]+TX, pefurazoate [101903-30-4]+TX, penconazole [66246-88-6]+TX, prothioconazole [178928-70-6]+TX, pyrifenox [88283-41-4]+TX, prochloraz [67747-09-5]+TX, propiconazole [60207-90-1]+TX, simeconazole [149508-90-7]+TX, tebuconazole [107534-96-3]+TX, tetraconazole [112281-77-3]+TX, triadimefon [43121-43-3]+TX, triadimenol [55219-65-3]+TX, triflumizole [99387-89-0]+TX, triticonazole [131983-72-7]+TX, ancymidol [12771-68-5]+TX, fenarimol [60168-88-9]+TX, nuarimol [63284-71-9]+TX, bupirimate [41483-43-6]+TX, dimethirimol [5221-53-4]+TX, ethirimol [23947-60-6]+TX, dodemorph [1593-77-7]+TX, fenpropidine [67306-00-7]+TX, fenpropimorph [67564-91-4]+TX, spiroxamine [118134-30-8]+TX, tridemorph [81412-43-3]+TX, cyprodinil [121552-61-2]+TX, mepanipyrim [110235-47-7]+TX, pyrimethanil [53112-28-0]+TX, fenpiclonil [74738-17-3]+TX, fludioxonil [131341-86-1]+TX, benalaxyl [71626-11-4]+TX, furalaxyl [57646-30-7]+TX, metalaxyl [57837-19-1]+TX, R-metalaxyl [70630-17-0]+TX, ofurace [58810-48-3]+TX, oxadixyl [77732-09-3]+TX, benomyl [17804-35-2]+TX, carbendazim [10605-21-7]+TX, debacarb [62732-91-6]+TX, fuberidazole [3878-19-1]+TX, thiabendazole [148-79-8]+TX, chlozolinate [84332-86-5]+TX, dichlozoline [24201-58-9]+TX, iprodione [36734-19-7]+TX, myclozoline [54864-61-8]+TX, procymidone [32809-16-8]+TX, vinclozoline [50471-44-8]+TX, boscalid [188425-85-6]+TX, carboxin [5234-68-4]+TX, fenfuram [24691-80-3]+TX, flutolanil [66332-96-5]+TX, mepronil [55814-41-0]+TX, oxycarboxin [5259-88-1]+TX, penthiopyrad [183675-82-3]+TX, thifluzamide [130000-40-7]+TX, guazatine [108173-90-6]+TX, dodine [2439-10-3][112-65-2] (free base)+TX, iminoctadine [13516-27-3]+TX, azoxystrobin [131860-33-8]+TX, dimoxystrobin [149961-52-4]+TX, enestroburin {Proc. BCPC, Int. Congr., Glasgow, 2003, 1, 93}+TX, fluoxastrobin [361377-29-9]+TX, kresoxim-methyl [143390-89-0]+TX, metominostrobin [133408-50-1]+TX, trifloxystrobin [141517-21-7]+TX, orysastrobin [248593-16-0]+TX, picoxystrobin [117428-22-5]+TX, pyraclostrobin [175013-18-0]+TX, ferbam [14484-64-1]+TX, mancozeb [8018-01-7]+TX, maneb [12427-38-2]+TX, metiram [9006-42-2]+TX, propineb [12071-83-9]+TX, thiram [137-26-8]+TX, zineb [12122-67-7]+TX, ziram [137-30-4]+TX, captafol [2425-06-1]+TX, captan [133-06-2]+TX, dichlofluanid [1085-98-9]+TX, fluoroimide [41205-21-4]+TX, folpet [133-07-3]+TX, tolylfluanid [731-27-1]+TX, bordeaux mixture [8011-63-0]+TX, copperhydroxid [20427-59-2]+TX, copperoxychlorid [1332-40-7]+TX, coppersulfat [7758-98-7]+TX, copperoxid [1317-39-1]+TX, mancopper [53988-93-5]+TX, oxine-copper [10380-28-6]+TX, dinocap [131-72-6]+TX, nitrothal-isopropyl [10552-74-6]+TX, edifenphos [17109-49-8]+TX, iprobenphos [26087-47-8]+TX, isoprothiolane [50512-35-1]+TX, phosdiphen [36519-00-3]+TX, pyrazophos [13457-18-6]+TX, tolclofos-methyl [57018-04-9]+TX, acibenzo-lar-S-methyl [135158-54-2]+TX, anilazine [101-05-3]+TX, benthiavalicarb [413615-35-7]+TX, blasticidin-S [2079-00-7]+TX, chinomethionat [2439-01-2]+TX, chloroneb [2675-77-6]+TX, chlorothalonil [1897-45-6]+TX, cyflufenamid [180409-60-3]+TX, cymoxanil [57966-95-7]+TX, dichlone [117-80-6]+TX, diclocymet [139920-32-4]+TX, diclomezine [62865-36-5]+TX, dicloran [99-30-9]+TX, diethofencarb [87130-20-9]+TX, dimethomorph [110488-70-5]+TX, SYP-L190 (Flumorph) [211867-47-9]+TX, dithianon [3347-22-6]+TX, ethaboxam [162650-77-3]+TX, etridiazole [2593-15-9]+TX, famoxadone [131807-57-3]+TX, fenamidone [161326-34-7]+TX, fenoxanil [115852-48-7]+TX, fentin [668-34-8]+TX, ferimzone [89269-64-7]+TX, fluazinam [79622-59-6]+TX, fluopicolide [239110-15-7]+TX, flusulfamide [106917-52-6]+TX, fenhexamid [126833-17-8]+TX, fosetyl-aluminium [39148-24-8]+TX, hymexazol [10004-44-1]+TX, iprovalicarb [140923-17-7]+TX, IKF-916 (Cyazofamid) [120116-88-3]+TX, kasugamycin [6980-18-3]+TX, methasulfocarb [66952-49-6]+TX, metrafenone [220899-03-6]+TX, pencycuron [66063-05-6]+TX, phthalide [27355-22-2]+TX, polyoxins [11113-80-7]+TX, probenazole [27605-76-1]+TX, propamocarb [25606-41-1]+TX, proquinazid [189278-12-4]+TX, pyroquilon [57369-32-1]+TX, quinoxyfen [124495-18-7]+TX, quintozene [82-68-8]+TX, sulfur [7704-34-9]+TX, tiadinil [223580-51-6]+TX, triazoxide [72459-58-6]+TX, tricyclazole [41814-78-2]+TX, triforine [26644-46-2]+TX, validamycin [37248-47-8]+TX, zoxamide (RH7281) [156052-68-5]+TX, mandipropamid [374726-62-2]+TX, isopyrazam [881685-58-1]+TX, sedaxane [874967-67-6]+TX, 3-difluoromethyl-1-methyl-1H-pyrazole-4-carboxylic acid (9-dichloromethylene-1,2,3,4-tetrahydro-1,4-methano-naphthalen-5-yl)-amide (dislosed in WO 2007/048556)+TX, 3-difluoromethyl-1-methyl-1H-pyrazole-4-carboxylic acid (3′,4′,5′-trifluoro-biphenyl-2-yl)-amide (disclosed in WO 2006/087343)+TX, [(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11Hnaphtho[2,1-b]pyrano[3,4-e]pyran-4-yl]methyl-cyclopropanecarboxylate [915972-17-7]+TX and 1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide [926914-55-8]+TX, and

microbials including: Acinetobacter lwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K₈₄ (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®)+TX, Bacillus circulans+TX, Bacillus firmus (BioSafe®, BioNem-WP®, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis Cry1Ab+TX, Bacillus thuringiensis aizawai GC 91 (Agree®)+TX, Bacillus thuringiensis israelensis (BMP123®+TX, Aquabac®+TX, VectoBac®)+TX, Bacillus thuringiensis kurstaki (Javelin®+TX, Deliver®+TX, CryMax®+TX, Bonide®+TX, Scutella WP®+TX, Turilav WP®+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD#32+TX, Bacillus thuringiensis strain AQ52+TX, Bacillus thuringiensis var. aizawai (XenTari®+TX, DiPel®)+TX, bacteria spp. (GROWMEND®+TX, GROWSWEET®+TX, Shootup®)+TX, bacteriophage of Clavipacter michiganensis (AgriPhage®)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmo-miniatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®/Biofox C®)+TX, Fusarium proliferatum+TX, Fusarium spp.+TX, Galactomyces geotrichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone-formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria Dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®+TX, Root Maximizer®)+TX, Myrothecium verrucaria strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C₉-1®)+TX, Pantoea spp.+TX, Pasteuria spp. (Econem®)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomons fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®+TX, Polyversum®)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces ga/bus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®+TX, PlantShield HC®+TX, RootShield®+TX, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichodermalongibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Verticillium lecanii (Mycotal®+TX, Vertalec®)+TX, Vip3Aa20 (VlPtera®)+TX, Virgibaclillus marismortui+TX, Xanthomonas campestris pv. Poae (Camperico®)+TX, Xenorhabdus bovienii+TX, Xenorhabdus nematophilus; and

Plant extracts including: pine oil (Retenol®)+TX, azadirachtin (Plasma Neem Oil®+TX, AzaGuard®+TX, MeemAzal®+TX, Molt-X®+TX, Botanical IGR (Neemazad®, Neemix®)+TX, canola oil (Lilly Miller Vegol®)+TX, Chenopodium ambrosioides near ambrosioides (Requiem®)+TX, Chrysanthemum extract (Crisant®)+TX, extract of neem oil (Trilogy®)+TX, essentials oils of Labiatae (Botania®)+TX, extracts of clove rosemary peppermint and thyme oil (Garden Insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaC20)+TX, Reynoutria sachalinensis (Regalia®+TX, Sakalia®)+TX, rotenone (Eco Roten®)+TX, Rutaceae plant extract (Soleo®)+TX, soybean oil (Ortho Ecosense®)+TX, tea tree oil (Timorex Gold®)+TX, thymus oil+TX, AGNIQUE® MMF+TX, BugOil®+TX, mixture of rosemary sesame pepermint thyme and cinnamon extracts (EF 300®)+TX, mixture of clove rosemary and peppermint extract (EF 400®)+TX, mixture of clove pepermint garlic oil and mint (Soil Shot®)+TX, kaolin (Screen®)+TX, storage glucam of brown algae (Laminarin®)+TX; and

pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC-LR Sprayable Pheromone®)+TX, Muscamone (Snip7 Fly Bait®+TX, Starbar Premium Fly Bait®)+TX, Oriental Fruit Moth Pheromone (3M oriental fruit moth sprayable Pheromone®)+TX, Peachtree Borer Pheromone (Isomate-P0)+TX, Tomato Pinworm Pheromone (3M Sprayable Pheromone®)+TX, Entostat powder (extract from palm tree) (Exosex CM®)+TX, Tetradecatrienyl acetate+TX, 13-Hexadecatrienal+TX, (E+TX,Z)-7+TX,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate; and

Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®+TX, Andersoni-System®)+TX, Amblyseius califomicus (Amblyline®+TX, Spical®)+TX, Amblyseius cucumeris (Thripex®+TX, Bugline cucumeris®)+TX, Amblyseius fallacis (Fallacis®)+TX, Amblyseius swirskii (Bugline swirskii®+TX, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®+TX, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripol®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®+TX, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®+TX, Digline®)+TX, Dacnusa sibirica (DacDigline®+TX, Minex®)+TX, Diversinervus spp.+TX, Encarsia citrina+TX, Encarsia formosa (Encarsia Max®+TX, Encarline®+TX, En-Strip®)+TX, Eretmocerus eremicus (Enermix®)+TX, Encarsia guade/oupae+TX, Encarsia haitiensis+TX, Episyrphus balteatus (Syrphidend®)+TX, Eretmoceris siphonini+TX, Eretmocerus califomicus+TX, Eretmocerus eremicus (Ercal®+TX, Eretline E®)+TX, Eretmocerus eremicus (Bemimix®)+TX, Eretmocerus hayati+TX, Eretmocerus mundus (Bemipar®+TX, Eretline M®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanem®+TX, B-Green®+TX, NemAttack®+TX, Nematop®)+TX, Heterorhabditis megidis (Nemasys H®+TX, BioNem H®+TX, Exhibitline hm®+TX, Larvanem-M®)+TX, Hippodamia convergens+TX, Hypoaspis aculeifer (Aculeifer-System®+TX, Entomite-A®)+TX, Hypoaspis miles (Hypoline m®+TX, Entomite-M®)+TX, Lbalia/eucospoides+TX, Lecanoideus floccissimus+TX, Lemophagus errabundus+TX, Leptomastidea abnormis+TX, Leptomastix dactylopfi (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline c®+TX, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEXC))+TX, Neoseiulus fallacis+TX, Nesideocoris tenuis (NesidioBug®+TX, Nesibug®)+TX, Ophyra aenescens (Biofly®)+TX, Orius insidiosus (Thripor-I®+TX, Oriline i®)+TX, Orius laevigatus (Thripor-L®+TX, Oriline I®)+TX, Orius majusculus (Oriline m®)+TX, Orius strigicollis (Thripor-S®)+TX, Pauesia juniperorum+TX, Pediobius foveo/atus+TX, Phasmarhabditis hermaphrodita (Nemaslug®)+TX, Phymastichus coffea+TX, Phytoseiulus macropilus+TX, Phytoseiulus persimilis (Spidex®+TX, Phytoline p®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+

TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®)+TX, Steinernema feltiae (NemaShield®+TX, Nemasys F®+TX, BioNem F®+TX, Steinernema-System®+TX, NemAttack®+TX, Nemaplus®+TX, Exhibitline sf®+TX, Scia-Rid®+TX, Entonem®)+TX, Steinernema kraussei (Nemasys L®+TX, BioNem L®+TX, Exhibitline srb®)+TX, Steinernema riobrave (BioVector®+TX, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine b®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimp/a stemmator; and

other biologicals including: abscisic acid+TX, bioSea®+TX, Chondrostereum purpureum (Chontrol Paste®)+TX, Colletotrichum gloeosporioides (Collego®)+TX, Copper Octanoate (Cueva0®)+TX, Delta traps (Trapline d®)+TX, Erwinia amylovora (Harpin) (ProAct®+TX, Ni-HIBIT Gold CST®)+TX, Ferri-phosphate (Ferramol®)+TX, Funnel traps (Trapline y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline f®)+TX, Microctonus hyperodae+TX, Mycoleptodiscus terrestris (Des-X®)+TX, BioGain®+TX, Aminomite®+TX, Zenox®+TX, Pheromone trap (Thripline ams®)+TX, potassium bicarbonate (MilStop®)+TX, potassium salts of fatty acids (Sanova®)+TX, potassium silicate solution (SD-Matrix®)+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+b®)+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.

The active ingredient mixture of the compounds of formula I selected from Tables 1, 2 and P with active ingredients described above comprises a compound selected from Tables 1, 2 and P and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula I selected from Tables 1, 2 and P and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula I selected from Tables 1, 2 and P and the active ingredients as described above is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula I. The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula (I). Further, it is hereby made available, a composition comprising a plant propagation material treated with a compound of formula (I).

Seed treatment comprises all suitable seed treatment techniques known in the art, such as seed dressing, seed coating, seed dusting, seed soaking and seed pelleting. The seed treatment application of the compound formula (I) can be carried out by any known methods, such as spraying or by dusting the seeds before sowing or during the sowing/planting of the seeds.

### BIOLOGICAL EXAMPLES

Example B1: Activity against Spodoptera littoralis (Egyptian cotton leaf worm) Cotton leaf discs were placed onto agar in 24-well microtiter plates and sprayed with aqueous test solutions prepared from 10′000 ppm DMSO stock solutions. After drying the leaf discs were infested with five L1 larvae. The samples were assessed for mortality, anti-feeding effect, and growth inhibition in comparison to untreated samples 3 days after infestation. Control of Spodoptera littoralis by a test sample is given when at least one of the categories mortality, anti-feedant effect, and growth inhibition is higher than the untreated sample.

The following compounds resulted in at least 80% control at an application rate of 200 ppm: P2, P3, P4, P5, P6, P7, P8, P9, P10, P12, P15, P16, P18, P19, P20, P22, P23, P24 and P25.

Example B2: Activity against Spodoptera littoralis (Egyptian cotton leaf worm) Test compounds were applied by pipette from 10′000 ppm DMSO stock solutions into 24-well plates and mixed with agar. Lettuce seeds were placed onto the agar and the multi well plate was closed by another plate which contained also agar. After 7 days the compound was absorbed by the roots and the lettuce grew into the lid plate. The lettuce leaves were then cut off into the lid plate. Spodoptera eggs were pipetted through a plastic stencil onto a humid gel blotting paper and the lid plate was closed with it. The samples were assessed for mortality, anti-feedant effect and growth inhibition in comparison to untreated samples 6 days after infestation.

The following compound gave an effect of at least 80% in at least one of the three categories (mortality, anti-feeding, or growth inhibition) at a test rate of 12.5 ppm: P3, P15, P16, P19 and P22.

### Example B3: Activity Against Plutella xylostella (Diamond Back Moth)

24-well microtiter plates with artificial diet were treated with aqueous test solutions prepared from 10′000 ppm DMSO stock solutions by pipetting. After drying, the plates were infested with L2 larvae (10 to 15 per well). The samples were assessed for mortality and growth inhibition in comparison to untreated samples 5 days after infestation.

The following compounds gave an effect of at least 80% in at least one of the two categories (mortality or growth inhibition) at an application rate of 200 ppm: P3, P5, P6, P8, P9, P10, P12, P15, P16, P18, P19, P20, P22, P23, P24 and P25.

### Example B4: Activity Against Diabrotica Balteata (Corn Root Worm)

Maize sprouts, placed onto an agar layer in 24-well microtiter plates were treated with aqueous test solutions prepared from 10′000 ppm DMSO stock solutions by spraying. After drying, the plates were infested with L2 larvae (6 to 10 per well). The samples were assessed for mortality and growth inhibition in comparison to untreated samples 4 days after infestation.

The following compounds gave an effect of at least 80% in at least one of the two categories (mortality or growth inhibition) at an application rate of 200 ppm: P2, P3, P5, P8, P9, P12, P15, P16, P18, P19, P20, P22, P23, P24 and P25.

### Example B5: Activity Against Myzus persicae (Green Peach Aphid)

Sunflower leaf discs were placed onto agar in a 24-well microtiter plate and sprayed with aqueous test solutions prepared from 10′000 ppm DMSO stock solutions. After drying, the leaf discs were infested with an aphid population of mixed ages. The samples were assessed for mortality 6 days after infestation.

The following compounds resulted in at least 80% mortality at an application rate of 200 ppm: P3, P8, P9, P14, P15, P16, P18, P19, P22, P23, P24 and P25.

### Example B6: Activity Against Myzus persicae (Green Peach Aphid)

Roots of pea seedlings infested with an aphid population of mixed ages were placed directly in the aqueous test solutions prepared from 10′000 DMSO stock solutions. The samples were assessed for mortality 6 days after placing seedlings in test solutions.

The following compounds resulted in at least 80% mortality at a test rate of 24 ppm: P16.

### Example B7: Activity Against Bemisia tabaci (Cotton White Fly)

Cotton leaf discs were placed onto agar in 24-well microtiter plates and sprayed with aqueous test solutions prepared from 10′000 ppm DMSO stock solutions. After drying the leaf discs were infested with adult white flies. The samples were checked for mortality 6 days after incubation.

The following compounds resulted in at least 80% mortality at an application rate of 200 ppm: P11 and P25.

### Example B8: Activity Against Euschistus heros (Neotropical Brown Stink Bug)

Soybean leaves on agar in 24-well microtiter plates were sprayed with aqueous test solutions prepared from 10′000 ppm DMSO stock solutions. After drying the leaves were infested with N2 nymphs. The samples were assessed for mortality and growth inhibition in comparison to untreated samples 5 days after infestation.

The following compounds gave an effect of at least 80% in at least one of the two categories (mortality or growth inhibition) at an application rate of 200 ppm: P2, P3, P5, P9, P15, P16, P18, P19, P20, P23, P24 and P25.

1. A compound of formula I 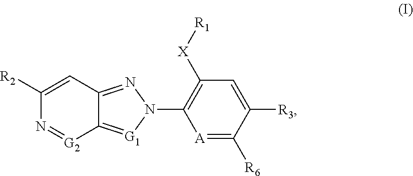
wherein
A is CH or N;
X is S, SO or SO₂;
R₁ is C₁-C₆alkyl, C₁-C₄haloalkyl, C₃-C₆cycloalkyl or C₃-C₆cycloalkyl-C₁-C₄alkyl; or
R₁ is C₃-C₆cycloalkyl mono- or polysubstituted by substituents independently selected from the group consisting of C₁-C₄alkyl, C₁-C₄haloalkyl, halogen and cyano; or
R₁ is C₃-C₆cycloalkyl-C₁-C₄alkyl mono- or polysubstituted by substituents independently selected from the group consisting of C₁-C₄alkyl, C₁-C₄haloalkyl, halogen and cyano;
R₂ is hydrogen, halogen, cyano, C₁-C₆haloalkyl, C₁-C₄haloalkylsulfanyl, C₁-C₄haloalkylsulfinyl, C₁-C₄haloalkylsulfonyl, C₁-C₄haloalkoxy or —C(O)(C₁-C₄haloalkyl); or
R₂ is C₃-C₆cycloalkyl which can be mono- or polysubstituted by substituents independently selected from the group consisting of C₁-C₄alkyl, C₁-C₄haloalkyl, halogen and cyano;
R₃ is hydrogen, C₁-C₄alkyl, C₁-C₄haloalkyl, halogen or cyano;
G₁ is CR₄, wherein R₄ is hydrogen, C₁-C₄alkyl, C₁-C₄haloalkyl, cyano or halogen;
G₂ is N or CR₅, wherein R₅ is hydrogen, C₁-C₄alkyl, C₁-C₄haloalkyl, cyano, nitro or halogen;
R₆ is amino, NHOH, NR₇R₈, C₁-C₆alkoxy, C₁-C₄alkoxyC₁-C₄alkyl, C₃-C₆cycloalkyl, C₃-C₆cycloalkyl-C₁-C₄alkyl, C₂-C₆alkenyl, C₂-C₆haloalkenyl, C₂-C₆alkynyl, C₂-C₆haloalkynyl, C₁-C₆haloalkylsulfanyl, C₁-C₆haloalkylsulfinyl, C₁-C₆haloalkylsulfonyl, C₁-C₆ haloalkoxy, —C(O)C₁-C₄haloalkyl, C₁-C₆alkylsulfanyl, C₁-C₆alkylsulfinyl, or C₁-C₆alkylsulfonyl; or
R₆ is C₃-C₆cycloalkyl which is mono- or di-substituted by substituents selected from the group consisting of halogen, C₁-C₄alkyl, C₁-C₄haloalkyl and cyano; or
R₆ is phenyl which can be mono- or polysubstituted by substituents selected from the group consisting of halogen, cyano, nitro, C₂-C₆alkenyl, C₂-C₆haloalkenyl, C₂-C₆alkynyl, C₂-C₆haloalkynyl, C₁-C₄alkyl, C₁-C₄haloalkyl, C₁-C₄haloalkoxy, C₁-C₄alkoxy, C₁-C₄alkoxy-C₁-C₄alkyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl, C₁-C₄alkylsulfonyl, and —C(O)C₁-C₄haloalkyl; or
R₆ is pyrimidinyl which can be mono- or polysubstituted by substituents selected from the group consisting of halogen, cyano, nitro, C₂-C₆alkenyl, C₂-C₆haloalkenyl, C₂-C₆alkynyl, C₂-C₆haloalkynyl, C₁-C₄alkyl, C₁-C₄haloalkyl, C₁-C₄haloalkoxy, C₁-C₄alkoxy, C₁-C₄alkoxy-C₁-C₄alkyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl, C₁-C₄alkylsulfonyl and —C(O)C₁-C₄haloalkyl; or
R₆ is pyridinyl which can be mono- or polysubstituted by substituents selected from the group consisting of halogen, cyano, nitro, C₂-C₆alkenyl, C₂-C₆haloalkenyl, C₂-C₆alkynyl, C₂-C₆haloalkynyl, C₁-C₄alkyl, C₁-C₄haloalkyl, C₁-C₄haloalkoxy, C₁-C₄alkoxy, C₁-C₄alkoxy-C₁-C₄alkyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl, or C₁-C₄alkylsulfonyl and —C(O)C₁-C₄haloalkyl; or
R₆ is a five- to six-membered, aromatic, partially saturated or fully saturated ring system linked via a nitrogen ring atom to the ring which contains the substituent X—R₁, said ring system can be mono- or polysubstituted by substituents selected from the group consisting of halogen, cyano, nitro, C₂-C₆alkenyl, C₂-C₆haloalkenyl, C₂-C₆alkynyl, C₂-C₆haloalkynyl, C₁-C₄alkyl, C₁-C₄haloalkyl, C₁-C₄haloalkoxy, C₁-C₄alkoxy, C₁-C₄alkoxy C₁-C₄alkyl, C₁-C₄alkylsulfanyl, C₁-C₄alkylsulfinyl, C₁-C₄alkylsulfonyl, —C(O)C₁-C₄alkyl, C₁-C₄haloalkylsulfanyl, C₁-C₄haloalkylsulfinyl, C₁-C₄haloalkylsulfonyl and —C(O)C₁-C₄haloalkyl; and said ring system contains 1, 2 or 3 heteroatoms selected from the group consisting of nitrogen, oxygen and sulphur, where said ring system may not contain more than one oxygen atom and not more than one sulfur atom;
R₇ is C₁-C₄alkyl, C₁-C₄haloalkyl, C₁-C₄haloalkoxy, C₁-C₄alkoxy or is C₃-C₆cycloalkyl which is mono- or di-substituted by substituents independently selected from the group consisting of halogen, C₁-C₄alkyl, C₁-C₄haloalkyl and cyano; or
R₇ is —C(O)C₁-C₄alkyl, —C(O)C₁-C₄haloalkyl or —C(O)C₂-C₆cycloalkyl; and
R₈ is hydrogen, C₁-C₄alkyl, C₁-C₄haloalkyl, C₁-C₄haloalkoxy, C₁-C₄alkoxy or is C₃-C₆cycloalkyl which is mono- or di-substituted by substituents independently selected from the group consisting of halogen, C₁-C₄alkyl, C₁-C₄haloalkyl and cyano; or
R₈ is —C(O)C₁-C₄alkyl, —C(O)C₁-C₄haloalkyl or —C(O)C₂-C₆cycloalkyl;
and agrochemically acceptable salts, stereoisomers, enantiomers, tautomers and N-oxides of the compounds of formula I.

2. A compound according to claim 1, represented by the compounds of formula I-1 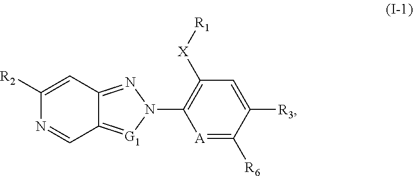
wherein the substituents X, R₁, R₂, R₃, R₆, G₁ and A are as defined under formula I in claim 1.

3. A compound of formula I-1 according to claim 2, wherein
R₁ is C₁-C₄alkyl, C₃-C₆cycloalkyl-C₁-C₄alkyl or C₃-C₆cycloalkyl; and
R₂ is halogen, C₁-C₄haloalkylsulfanyl, C₁-C₄haloalkylsulfinyl, C₁-C₄haloalkylsulfonyl, C₁-C₄haloalkoxy, C₁-C₄haloalkyl, cyano or is C₃-C₆cycloalkyl which can be mono- or polysubstituted by substituents independently selected from the group consisting of halogen, C₁-C₄ haloalkyl, cyano and C₁-C₄alkyl.

4. A compound according to claim 1, represented by the compounds of formula I-1a 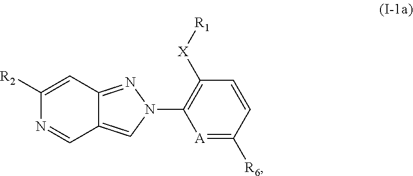
wherein
R₁ is C₁-C₄alkyl, C₃-C₆cycloalkyl-C₁-C₄alkyl or C₃-C₆cycloalkyl;
R₂ is halogen, C₁-C₄haloalkylsulfanyl, C₁-C₄haloalkylsulfinyl, C₁-C₄haloalkylsulfonyl, C₁-C₄haloalkoxy, C₁-C₄haloalkyl, cyano or is C₃-C₆cycloalkyl which can be mono- or polysubstituted by substituents independently selected from the group consisting of halogen, C₁-C₄ haloalkyl, cyano and C₁-C₄alkyl.

5. A compound of formula I-1a according to claim 4, wherein
R₁ is C₁-C₄alkyl, C₃-C₆cycloalkyl-C₁-C₄alkyl or C₃-C₆cycloalkyl;
R₂ is halogen, C₁-C₄haloalkylsulfanyl, C₁-C₄haloalkylsulfinyl, C₁-C₄haloalkylsulfonyl, C₁-C₄haloalkoxy, C₁-C₄haloalkyl, cyano or is C₃-C₆cycloalkyl which can be mono- or polysubstituted by substituents independently selected from the group consisting of halogen, C₁-C₄ haloalkyl, cyano and C₁-C₄alkyl;
and R₆ is selected from the group J consisting of 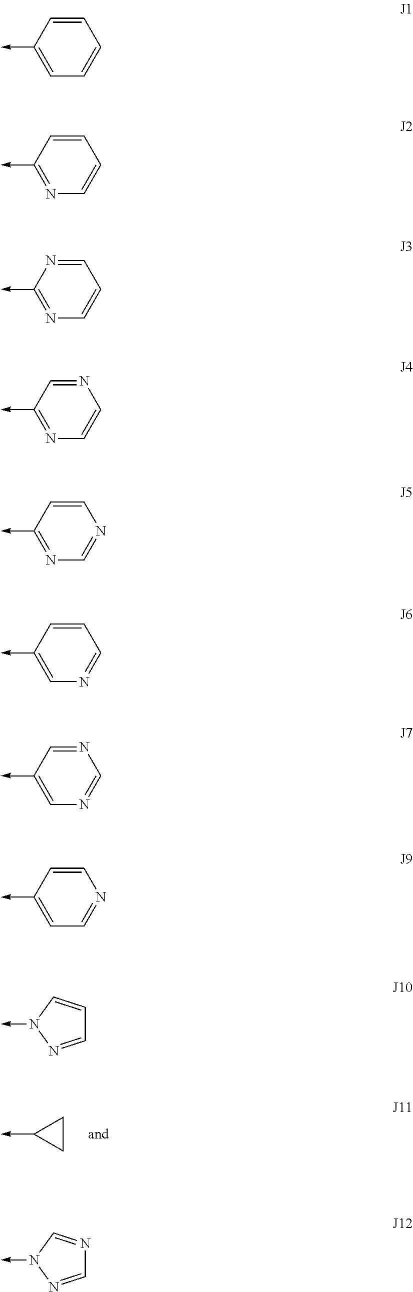
wherein each of the groups J1 to J10 and J12 independently can be mono- or di-substituted by substituents independently selected from the group consisting of halogen, cyano, nitro, C₂-C₆alkenyl, C₂-C₆haloalkenyl, C₂-C₆alkynyl, C₂-C₆haloalkynyl, C₁-C₄alkyl, C₁-C₄haloalkyl, C₁-C₄haloalkoxy, C₁-C₄alkoxy, C₁-C₄alkoxy C₁-C₄alkyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl and C₁-C₄alkylsulfonyl; and J11 can be substituted by halogen, C₁-C₄alkyl, C₁-C₄haloalkyl or cyano; or R₆ is amino; or R₆ is NHOH.

6. A compound according to claim 1, represented by the compounds of formula I-2 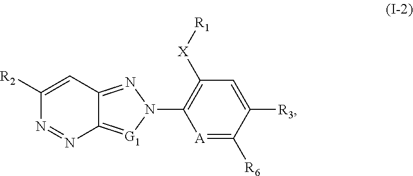
wherein the substituents X, R₁, R₂, R₃, R₆, G₁ and A are as defined under formula I in claim 1.

7. A compound of formula I-2 according to claim 6, wherein
R₁ is C₁-C₄alkyl, C₃-C₆cycloalkyl-C₁-C₄alkyl or C₃-C₆cycloalkyl; and
R₂ is halogen, C₁-C₄haloalkylsulfanyl, C₁-C₄haloalkylsulfinyl, C₁-C₄haloalkylsulfonyl, C₁-C₄haloalkoxy, C₁-C₄haloalkyl, cyano or is C₃-C₆cycloalkyl which can be mono- or polysubstituted by substituents independently selected from the group consisting of halogen, C₁-C₄ haloalkyl, cyano and C₁-C₄alkyl.

8. A compound according to claim 1, represented by the compounds of formula I-2a 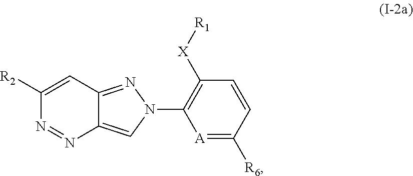
wherein
R₁ is C₁-C₄alkyl, C₃-C₆cycloalkyl-C₁-C₄alkyl or C₃-C₆cycloalkyl;
R₂ is halogen, C₁-C₄haloalkylsulfanyl, C₁-C₄haloalkylsulfinyl, C₁-C₄haloalkylsulfonyl, C₁-C₄haloalkoxy, C₁-C₄haloalkyl, cyano or is C₃-C₆cycloalkyl which can be mono- or polysubstituted by substituents independently selected from the group consisting of halogen, C₁-C₄ haloalkyl, cyano and C₁-C₄alkyl.

9. A compound of formula I-2a according to claim 8, wherein
R₁ is C₁-C₄alkyl, C₃-C₆cycloalkyl-C₁-C₄alkyl or C₃-C₆cycloalkyl;
R₂ is halogen, C₁-C₄haloalkylsulfanyl, C₁-C₄haloalkylsulfinyl, C₁-C₄haloalkylsulfonyl, C₁-C₄haloalkoxy, C₁-C₄haloalkyl, cyano or is C₃-C₆cycloalkyl which can be mono- or polysubstituted by substituents independently selected from the group consisting of halogen, C₁-C₄ haloalkyl, cyano and C₁-C₄alkyl;
and R₆ is selected from the group J consisting of 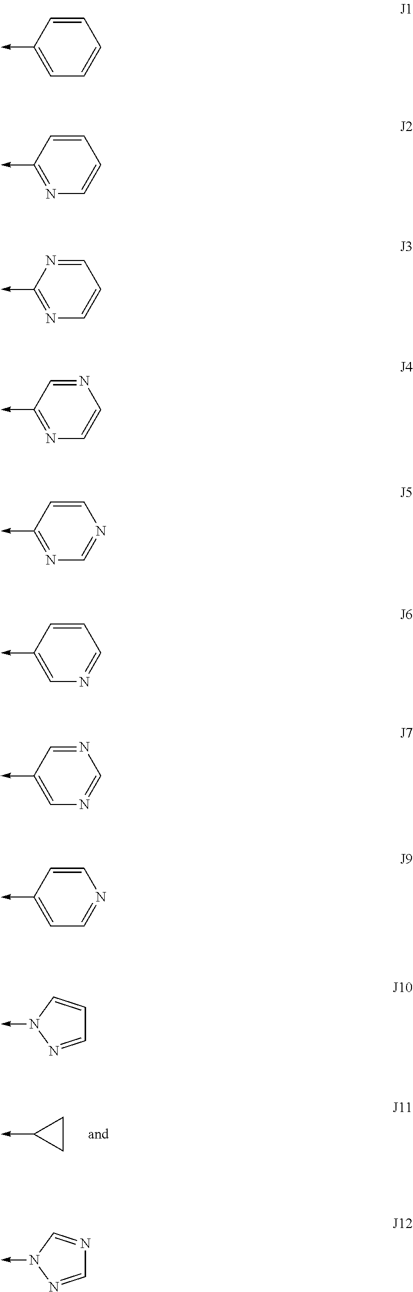
wherein each of the groups J1 to J10 and J12 independently can be mono- or di-substituted by substituents independently selected from the group consisting of halogen, cyano, nitro, C₂-C₆alkenyl, C₂-C₆haloalkenyl, C₂-C₆alkynyl, C₂-C₆haloalkynyl, C₁-C₄alkyl, C₁-C₄haloalkyl, C₁-C₄haloalkoxy, C₁-C₄alkoxy, C₁-C₄alkoxy C₁-C₄alkyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl and C₁-C₄alkylsulfonyl; and J11 can be substituted by halogen, C₁-C₄alkyl, C₁-C₄haloalkyl or cyano; or R₆ is amino.

10. A pesticidal composition, which comprises at least one compound of formula I according to claim 1 or, where appropriate, a tautomer thereof, in each case in free form or in agrochemically utilizable salt form, as active ingredient and at least one auxiliary.

11. A method for controlling pests, which comprises applying a composition according to claim 10 to the pests or their environment with the exception of a method for treatment of the human or animal body by surgery or therapy and diagnostic methods practised on the human or animal body.

12. A method for the protection of plant propagation material from the attack by pests, which comprises treating the propagation material or the site, where the propagation material is planted, with a composition according to claim 10.
